SEQUENCE LISTING (60 sequences)

SEQ ID NO: 1 - protein, 9 aa, Artificial Sequence
  VH-CDR1 of Antibody Kp3
SNSNTYYWG

SEQ ID NO: 2 - protein, 16 aa, Artificial Sequence
  VH-CDR2 of Antibody Kp3
TIHSSGRTYYNPSLKS

SEQ ID NO: 3 - protein, 22 aa, Artificial Sequence
  VH-CDR3 of Antibody Kp3
DLSGASLAPRRPFNYYYYNMDV

SEQ ID NO: 4 - protein, 5 aa, Artificial Sequence
  VH-CDR1 of Antibody Kp16
TYYMH

SEQ ID NO: 5 - protein, 17 aa, Artificial Sequence
  VH-CDR2 of Antibody Kp16
MINPSSGSTIYAQPFRG

SEQ ID NO: 6 - protein, 9 aa, Artificial Sequence
  VH-CDR3 of Antibody Kp16
GNYGSSFGY

SEQ ID NO: 7 - protein, 16 aa, Artificial Sequence
  VL-CDR1 of Antibody Kp3
RSSQSLVYSDGNTYLN

SEQ ID NO: 8 - protein, 7 aa, Artificial Sequence
  VL-CDR2 of Antibody Kp3
KVSNRDS

SEQ ID NO: 9 - protein, 10 aa, Artificial Sequence
  VL-CDR3 of Antibody Kp3
MQGTHWPPIT

SEQ ID NO: 10 - protein, 13 aa, Artificial Sequence
  VL-CDR1 of Antibody Kp16
SGSSSNIGSNTVN

SEQ ID NO: 11 - protein, 7 aa, Artificial Sequence
  VL-CDR2 of Antibody Kp16
NNNQRPS

SEQ ID NO: 12 - protein, 11 aa, Artificial Sequence
  VL-CDR3 of Antibody Kp16
AAWDDSLNGVV

SEQ ID NO: 13 - protein, 134 aa, Artificial Sequence
  VH Amino Acid Sequence of Antibody Kp3
QVQLQESGPGLVKPSETLSLTCTVSGGSMNSNSNTYYWGWIRQPPGKGLEWIGTIHSSGRTYYNPSLKSRVTISVDMSKNQFSLNLTSATAADTAVYYCARDLSGASLAPRRPFNYYYYNMDVWGRGTLVTVSS

SEQ ID NO: 14 - protein, 118 aa, Artificial Sequence
  VH Amino Acid Sequence of Antibody Kp16
QVQLQQSGAEVKKPGASVKVSCKASGYALTTYYMHWVRQAPGQGLQWMGMINPSSGSTIYAQPFRGRVTLTRDTSSGTVFMDLSSLTSEDTAIYYCARGNYGSSFGYWGKGTMVTVSS

SEQ ID NO: 15 - protein, 113 aa, Artificial Sequence
  VL Amino Acid Sequence of Antibody Kp3
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPITFGQGTRLEIK

SEQ ID NO: 16 - protein, 110 aa, Artificial Sequence
  VL Amino Acid Sequence of Antibody Kp16
SYVLTQPPSASGTPGQRVTISCSGSSSNIGSNTVNWYQQLPGTAPKLLIYNNNQRPSGVPDRFSGSKSGTSASLAISGLQSEDEADYYCAAWDDSLNGVVFGGGTKVTVL

SEQ ID NO: 17 - protein, 202 aa, Klebsiella pneumoniae
MKKVLLSAAMATAFFGMTAAHAADTTVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQ

SEQ ID NO: 18 - protein, 204 aa, Artificial Sequence
  consensus sequence of the MrkA proteins from all the types of Gram-negative bacteria of Sequences 19-25
  misc_feature (118)..(118): Xaa can be Ser or Ala or no amino acid
  misc_feature (169)..(169): Xaa can be Ala, Gln, or Thr
MAMKKVLLSAAMATAFFGMAAANAADTNVGGGQVNFFGKVTDVSCTVSVNGQGSDANVYLSPVTLTEVKAAAADTYLKPKSFTIDVSDCQAADGTKQDDVSKLGVNWTGGNLLAGATXKQQGYLANTEAAGAQNIQLVLSTDNATALTNKIIPGDSTQPKAKGDASAVXDGARFTYYVGYATSTPTTVTTGVVNSYATYEITYQ

SEQ ID NO: 19 - protein, 204 aa, Klebsiella pneumoniae
MAMKKVLLSAAMATAFFGMTAAHAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQ

SEQ ID NO: 20 - protein, 204 aa, Klebsiella oxytoca
MAMKKVLLSAAMATAFFGMAAANAADTNVGGGQVNFFGKVTDVSCTVSVNGQGSDANVYLSPVTLTEVKAAAADTYLKPKSFTIDVSDCQAADGTKQDDVSKLGVNWTGGNLLAGATAKQQGYLANTEAAGAQNIQLVLSTDNATALTNKIIPGDATQPKATGDASAVQDGARFTYYVGYATSTPTTVTTGVVNSYATYEITYQ

SEQ ID NO: 21 - protein, 202 aa, Salmonella montevideo
MKKVLLSAAMATAFFGMAAANAADTNVGGGQVNFFGKVTDVSCTVSVNGQGSDANVYLSPVTLTEVKAAAADTYLKPKSFTIDVSDCQAADGTKQDDVSKLGVNWTGGNLLSGATAKQQGYLANTEAAGAQNIQLVLSTDNATALTNKIIPGDSTQPKAAGDASAVQDGARFTYYVGYATSTPTTVTTGVVNSYATYEITYQ

SEQ ID NO: 22 - protein, 204 aa, Escherichia coli
MAMKKVLLSAAMATAFFGMAAANAADTNVGGGQVNFFGKVTDVSCTVSVNGQGSDANVYLSPVTLTEVKAAAADTYLKPKSFTIDVSDCQAADGTKQDDVSKLGVNWTGGNLLSGATAKQQGYLANTEAAGAQNIQLVLSTDNATALTNKIIPGDSTQPKAAGDASAVQDGARFTYYVGYATSTPTTVTTGVVNSYATYEITYQ

SEQ ID NO: 23 - protein, 202 aa, Shigella sp. LN126; identical to SEQ ID NO: 17

SEQ ID NO: 24 - protein, 186 aa, Enterobacter cloacae
MFNKTLIAAAIMFSGAAMAAESTGVAGGTITFNGSVSDTTCDVTTNNGSDFTVNLSPITLTDMGKTAGIVTANEKDFTMSLKNCTAADEGTKTLKITFTSSNLSDDGKYLKNYSEGGAEGVGITLTSDGKTAVPFDTAFNTGLTSDDVSSTDGITLTMHANYYNFGGASVTTGKVVTDATYSFSYD

SEQ ID NO: 25 - protein, 205 aa, Citrobacter freundii
MAMKKVLLSAAIATAFFGMAAANAADTNVGGGQVNFFGKVTDVSCTVSVNGQGSNADVYLAPVTLTEVKAAAADTYLKPKSFTIDVSDCQAADKTAQDDVSKLGVNWTGGNLLAGATSKQQGYLANTEAAGAQDIQLVLSTDTDTALTNKIIPNGSSAQPKAKVDTNAVANGARFTYYVGYVTSKPETVTAGVVNSYATYEITYQ

SEQ ID NO: 26 - protein, 162 aa, Artificial Sequence
  MrkA fragment
VSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQ

SEQ ID NO: 27 - protein, 170 aa, Artificial Sequence
  MrkA fragment
MKKVLLSAAMATAFFGMTAAHAADTTVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGA

SEQ ID NO: 28 - protein, 130 aa, Artificial Sequence
  MrkA fragment
VSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGA

SEQ ID NO: 29 - protein, 5 aa, Artificial Sequence
  st1_c1 “clone 1” VH-CDR1
SYAVH

SEQ ID NO: 30 - protein, 17 aa, Artificial Sequence
  st1_c1 “clone 1” VH-CDR2
GINGGNGNTRISQRFQD

SEQ ID NO: 31 - protein, 15 aa, Artificial Sequence
  st1_c1 “clone 1” VH-CDR3
ADDCSGVGCHPWFDP

SEQ ID NO: 32 - protein, 6 aa, Artificial Sequence
  st2_c4 “clone 4” VH-CDR1
NANWWS

SEQ ID NO: 33 - protein, 16 aa, Artificial Sequence
  st2_c4 “clone 4” VH-CDR2
EIYHSGTTYYNPSLKS

SEQ ID NO: 34 - protein, 12 aa, Artificial Sequence
  st2_c4 “clone 4” VH-CDR3
DRDITSRGTFDV

SEQ ID NO: 35 - protein, 5 aa, Artificial Sequence
  st3_c5 “clone 5” VH-CDR1
AYYMH

SEQ ID NO: 36 - protein, 17 aa, Artificial Sequence
  st3_c5 “clone 5” VH-CDR2
WINPSSGGTNSAQKFQG

SEQ ID NO: 37 - protein, 9 aa, Artificial Sequence
  st3_c5 “clone 5” VH-CDR3
GTIGAAGNY

SEQ ID NO: 38 - protein, 5 aa, Artificial Sequence
  st4_c6 “clone 6” VH-CDR1
SYAVH

SEQ ID NO: 39 - protein, 17 aa, Artificial Sequence
  st4_c6 “clone 6” VH-CDR2
GVNGGNGNTRFSQKFQD

SEQ ID NO: 40 - protein, 15 aa, Artificial Sequence
  st4_c6 “clone 6” VH-CDR3
ADDCSGVGCHPWFDP

SEQ ID NO: 41 - protein, 11 aa, Artificial Sequence
  st1_c1 “clone 1” VL-CDR1
SGDKLGDKYVS

SEQ ID NO: 42 - protein, 7 aa, Artificial Sequence
  st1_c1 “clone 1” VL-CDR2
KDTKRPS

SEQ ID NO: 43 - protein, 9 aa, Artificial Sequence
  st1_c1 “clone 1” VL-CDR3
QAWDRSIMI

SEQ ID NO: 44 - protein, 11 aa, Artificial Sequence
  st2_c4 “clone 4” VL-CDR1
RASEGIYHWLA

SEQ ID NO: 45 - protein, 7 aa, Artificial Sequence
  st2_c4 “clone 4” VL-CDR2
KASSLAS

SEQ ID NO: 46 - protein, 9 aa, Artificial Sequence
  st2_c4 “clone 4” VL-CDR3
QQYSNYPLT

SEQ ID NO: 47 - protein, 13 aa, Artificial Sequence
  st3_c5 “clone 5” VL-CDR1
SGSRPNIGGNTVN

SEQ ID NO: 48 - protein, 7 aa, Artificial Sequence
  st3_c5 “clone 5” VL-CDR2
SNSQRPS

SEQ ID NO: 49 - protein, 11 aa, Artificial Sequence
  st3_c5 “clone 5” VL-CDR3
AAWDDSLTGPV

SEQ ID NO: 50 - protein, 11 aa, Artificial Sequence
  st4_c6 “clone 6” VL-CDR1
SGDKLGDKYTS

SEQ ID NO: 51 - protein, 7 aa, Artificial Sequence
  st4_c6 “clone 6” VL-CDR2
QDTKRPS

SEQ ID NO: 52 - protein, 11 aa, Artificial Sequence
  st4_c6 “clone 6” VL-CDR3
QAWDSDSGTAT

SEQ ID NO: 53 - protein, 124 aa, Artificial Sequence
  st1_c1 “clone 1” VH Amino Acid Sequence
QVQLVQSGAEVRKPGASVTVFCRTSGYIFTSYAVHWVRQAPGQGLEWMGGINGGNGNTRISQRFQDRLMITRDRSANTASMELRSLTSEDTAIYYCARADDCSGVGCHPWFDPWGRGTLVTVSS

SEQ ID NO: 54 - protein, 121 aa, Artificial Sequence
  st2_c4 “clone 4” VH Amino Acid Sequence
QLQLQESGPGLVKPSGTLSLTCAVSGDSIDNANWWSWVRQTPGKGLEWIGEIYHSGTTYYNPSLKSRVTISIDNSKNQFSLALTSVTAADTAVYYCARDRDITSRGTFDVWGRGTMVTVSS

SEQ ID NO: 55 - protein, 118 aa, Artificial Sequence
  st3_c5 “clone 5” VH Amino Acid Sequence
QVQLVQSGAEVKKPGASLKVSCKASGYTFTAYYMHWVRQAPGHGLEWMGWINPSSGGTNSAQKFQGRVTMTRDTSINTAYMELSRLTSDDTAVYYCARGTIGAAGNYWGQGTLVTVSS

SEQ ID NO: 56 - protein, 124 aa, Artificial Sequence
  st4_c6 “clone 6” VH Amino Acid Sequence
QVQLVQSGAEVRKPGASVTLSCRTSGYTFTSYAVHWVRQAPGQGLEWMGGVNGGNGNTRFSQKFQDRLMIVRDRSANTASMELRSLTSEDTAVYYCARADDCSGVGCHPWFDPWGQGTLVTVSS

SEQ ID NO: 57 - protein, 106 aa, Artificial Sequence
  st1_c1 “clone 1” VL Amino Acid Sequence
QSVLTQPPSVSVSPGHTASITCSGDKLGDKYVSWYQQKSGQSPVLVMYKDTKRPSGIPERFSGSNSGNTATLAISGTQAVDEADYFCQAWDRSIMIFGGGTKVTVL

SEQ ID NO: 58 - protein, 107 aa, Artificial Sequence
  st2_c4 “clone 4” VL Amino Acid Sequence
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 59 - protein, 110 aa, Artificial Sequence
  st3_c5 “clone 5” VL Amino Acid Sequence
QSVLTQPPSASGTPGQRVTISCSGSRPNIGGNTVNWYQQLPGAAPKLLIYSNSQRPSGVPDRFSGSKYGTSASLAISGLQSDDEADYYCAAWDDSLTGPVFGGGTKLTIL

SEQ ID NO: 60 - protein, 108 aa, Artificial Sequence
  st4_c6 “clone 6” VL Amino Acid Sequence
SVILTQPPSVSVSPGQTANITCSGDKLGDKYTSWYLQKPGQSPVLLIFQDTKRPSDIPERFSGSNSGNTATLTISGTQAVDEADYYCQAWDSDSGTATFGGGTKLTVL

(19) United States
(12) Patent Application Publication
(10) Pub. No.: US 2019/0062411 A1
(43) Pub. Date: Feb. 28, 2019

(54) MRKA POLYPEPTIDES, ANTIBODIES, AND USES THEREOF

(71) Applicant: MedImmune, LLC, Gaithersburg, MD (US)

(72) Inventors: Qun WANG, Gaithersburg, MD (US); Saravanan RAJAN, Gaithersburg, MD (US); Chew-Shun CHANG, Gaithersburg, MD (US); Jenny HEIDBRINK THOMPSON, Gaithersburg, MD (US); Charles Kendall STOVER, Gaithersburg, MD (US); Meghan PENNINI, Gaithersburg, MD (US); William DALL'ACQUA, Gaithersburg, MD (US); Partha S. CHOWDHURY, Gaithersburg, MD (US); Xiaodong XIAO, Gaithersburg, MD (US); Hung-Yu LIN, Gaithersburg, MD (US)

(73) Assignee: MedImmune, LLC, Gaithersburg, MD (US)

(21) Appl. No.: 15/940,344

(22) Filed: Mar. 29, 2018

Related U.S. Application Data
(63) Continuation of application No. 15/244,960, filed on Aug. 23, 2016, now abandoned.
(60) Provisional application No. 62/238,828, filed on Oct. 8, 2015.
(60) Provisional application No. 62/208,975, filed on Aug. 24, 2015.

(51) Int. Cl. C07K 16/12; A61K 39/108; A61K 39/00

(52) U.S. Cl. CPC C07K 16/1228; A61K 39/0266; A61K 2039/55566; A61K 2039/545; C07K 2317/92; C07K 2317/76

(57) ABSTRACT

The present disclosure provides MrkA binding proteins, e.g., antibodies or antigen binding fragments thereof that bind to MrkA and induce opsonophagocytic killing of Klebsiella (e.g., Klebsiella pneumoniae). The present disclosure also provides methods of reducing Klebsiella (e.g., Klebsiella pneumoniae) or treating or preventing Klebsiella (e.g., Klebsiella pneumoniae) infection in a subject comprising administering MrkA binding proteins, e.g., antibodies or antigen-binding fragments thereof, MrkA polypeptides, immunogenic fragments thereof, or polynucleotides encoding MrkA or immunogenic fragments thereof to the subject.

### CROSS-REFERENCE TO RELATED APPLICATIONS

This application claims the benefit of U.S. Provisional Patent Application No. 62/208,975, filed Aug. 24, 2015, and U.S. Provisional Patent Application No. 62/238,828, filed Oct. 8, 2015, each of which is incorporated herein by reference in its entirety.

### REFERENCE TO SEQUENCE LISTING SUBMITTED ELECTRONICALLY

The content of the electronically submitted sequence listing in ASCII text file MRKA-100-WO-PCT_SeqListing.txt (Size: 42,157 bytes; and Date of Creation: Aug. 16, 2016) filed with the application is incorporated herein by reference in its entirety.

### BACKGROUND OF THE INVENTION

### Field of the Invention

The field of the invention generally relates to MrkA polypeptides, MrkA-encoding polynucleotides, and anti-MrkA antibodies for prevention or treatment of Klebsiella infections.

### Background of the Invention

Klebsiella is a Gram negative bacterium that is rapidly gaining clinical importance as a causative agent for optimistic and nosocomial infection, including pneumonia, urinary tract infection, neonatal septicemia, and surgery wound infection. In addition, there are emerging syndromes associated with Klebsiella infections such as pyogenic liver abscesses (PLA), endophthalmitis, meningitis, and necrotizing meningitis.

Over the last two decades, antibiotic resistance has emerged as one of the major challenges in the fight against bacterial infection. While some progress has been made against drug resistant Staphylococcus aureus, multi-drug resistant (MDR) Gram negative opportunistic infections are most problematic and call for novel antimicrobial drugs (see. e.g., Xu et al., Expert opinion on investigational drugs 2014; 23:163-82). Among these, Klebsiella pneumoniae, a causative agent for opportunistic and nosocomial infections (Broberg et al., F1000Prime Rep 2014; 6:64), has become particularly challenging with multi-drug resistant strains widely circulating. Klebsiella infections such as Extended-Spectrum Beta Lactamase (ESBL), K. pneumoniae carbapenemase (KPC), and New Delhi metallo-beta-lactamase 1 (NDM-1) have spread worldwide and rendered current antibiotic classes largely inadequate. This reality coupled with the dwindling antibiotics pipeline leaves clinicians with few therapeutic alternatives (Munoz-Price et al., Lancet Infect Dis 2013; 13:785-96). Several recent high profile outbreaks underscore the urgency associated with K. pneumoniae antibiotic resistance. See McKenna. Nature 2013; 499:394-6; or Snitkin et al., Sci Transl Med 2012; 4:148ra16. In addition, cross species spread of resistance indicates a need for alternative pathogen specific strategies, such as antibodies and vaccines, to complement or conserve antibiotics. Species-specific protective antibodies against bacterial infections would not be subject to the rapidly evolving antibiotic resistance mechanisms and preclinical data has demonstrated that they could also provide additional benefits to the patient in adjunctive use. See. e.g., DiGiandomenico et al., J Exp Med 2012; 209:1273-87; DiGiandomenico et al., Sci Transl Med 2014; 6:262ra155.

Multiple virulence factors have been implicated in K. pneumoniae pathogenesis (Podschun et al., Clin Microbiol Rev 1998; 11:589-603). The best characterized are capsular polysaccharides (CPS) and lipopolysaccharides (LPS). Polyclonal antibodies directed against LPS and CPS are protective in preclinical models of lethal K. pneumoniae infections (Ahmad et al., Vaccine 2012; 30:2411-20; Rukavina et al., Infect Immun 1997; 65:1754-60; Donta et al., J Infect Dis 1996; 174:537-43). However targeting these two antigens with antibodies or using them as immunogens in vaccine candidates poses a significant challenge with respect to strain coverage. There are more than seventy-seven known capsule serotypes and eight O-antigen serotypes, and it is not clear which are the most prevalent and/or associated with pathogenesis. Though serotype-specific monoclonal antibodies can confer protection against K. pneumoniae of defined LPS and capsular serotypes (Mandine et al., Infect Immun 1990; 58:2828-33), multivalent antigens and/or combination of antibodies are required for broad strain coverage and protection (Campbell et al., Clin Infect Dis 1996; 23:179-81). Identifying serotype independent, cross-protective antigens is still very challenging. For example, monoclonal antibodies which target conserved core LPS epitopes that are present across serotypes provided little to no protection in animal models (Brade et al. 2001, J Endotoxin Res, 7(2): 119-24).

Multiple strategies have been used in efforts to identify cross protective targets for K. pneumoniae, including genomics and proteomics approaches (Lundberg et al., Hum Vaccin Immunother 2012; 9:497-505; Meinke et al., Vaccine 2005; 23:2035-41; Maroncle et al., Infection and immunity 2002; 70:4729-34). Though a number of targets have been suggested from these studies, few have been validated through subsequent investigations. Of note, the majority of potential targets identified through such approaches are proteins involved in metabolic pathways which may not be suitable as antibody targets due to inaccessibility. Antigenome strategy represents a novel approach to identify directly antigens capable of eliciting antibody responses (Meinke et al. 2005, Vaccine, 23(17-18):2035-41). Its impact on K. pneumoniae investigation remains to be seen. Thus, there is a great need to identify and develop antibodies and/or immunogenic polypeptides/vaccines that have protective effect against K. pneumoniae infections.

### BRIEF SUMMARY OF THE INVENTION

The emergence and increasing cases of antibiotic resistant Klebsiella pneumoniae infections warrant the development of alternative approaches, such as antibody therapy and/or vaccines, for prevention and treatment. However, lack of validated targets that are shared by a spectrum of different clinical strains poses a significant challenge. A functional, target-agnostic screening approach was adopted to identify protective antibodies against novel targets. Several monoclonal antibodies were identified from phage display and hybridoma platforms via whole bacterial binding and screening for opsonophagocytic killing (OPK). Immunoprecipitation of K. pneumoniae cell lysate with antibodies possessing these activities followed by mass spectrometric analysis identified their target antigen to be MrkA, a major protein in type III fimbriae complex. Type III fimbriae mediate biofilm formation on biotic and abiotic surfaces and are required for mature biofilm development. The various components of type 3 fimbriae are encoded by the mrkABCDF operon, which produce the major pilin subunit MrkA, chaperone MrkB, outer membrane usher MrkC, adhesin MrkD and MrkF. See Yang et al. PLoS One. 2013 Nov. 14; 8(11):e79038. Host cell adherence and biofilm formation of Klebsiella are mediated by such MrkA pilins. See Chan et al., Langmuir 28: 7428-7435 (2012). These serotype independent MrkA antibodies also reduced biofilm formation and conferred protections in mouse pneumonia models. Importantly, mice immunized with purified MrkA proteins showed reduced organ burden upon K. pneumoniae infections. Accordingly, the present disclosure provides MrkA binding proteins, e.g., antibodies or antigen binding fragments thereof, that bind to and induce opsonophagocytic killing (OPK) of Klebsiella. The present disclosure also provides methods of treating Klebsiella infections using MrkA binding proteins, e.g., antibodies or antigen binding fragments thereof, MrkA polypeptides, immunogenic fragments thereof, and polynucleotides encoding MrkA polypeptides or immunogenic fragments thereof.

In one instance, provided herein is an isolated antigen binding protein that specifically binds to MrkA, wherein the antigen binding protein a) binds to at least two Klebsiella pneumoniae (K. pneumoniae) serotypes; b) induces opsonophagocytic killing (OPK) of K. pneumoniae or c) binds to at least two K. pneumoniae serotypes and induces OPK of K. pneumoniae. In one instance, the antigen binding protein binds to at least two K. pneumoniae serotypes selected from the group consisting of O1:K2, O1:K79, O2a:K28, O5:K57, O3:K58, O3:K11, O3:K25, O4:K15, O5:K61, O7:K67, and O12:K80. In one instance, the antigen binding protein induces OPK in at least one or two K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In one instance, the antigen binding protein induces 100% OPK in K. pneumoniae strains 9148 (O2a:K28), 9178 (O3:K58), and 9135 (O4:K15); and/or induces 80% OPK in K. pneumoniae strain 29011 (O1:K2) as measured using a bio-luminescent OPK assay. In one instance, the antigen binding protein confers survival benefit in an animal exposed to a K. pneumoniae strain selected from the group consisting of Kp29011, Kp9178, and Kp43816.

In one instance, provided herein is an isolated antigen binding protein that specifically binds to MrkA, wherein the antigen binding protein inhibits biofilm formation.

In one instance, provided herein is an isolated antigen binding protein that specifically binds to MrkA, wherein the antigen binding protein inhibits cell attachment.

In one instance, provided herein is an isolated antigen binding protein that specifically binds MrkA comprising a set of Complementarity-Determining Regions (CDRs): HCDR1, HCDR2, HCDR3, LCDR1, LCDR2, and LCDR3 wherein: HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 7; LCDR2 has the amino acid sequence of SEQ. ID. NO: 8; and LCDR3 has the amino acid sequence of SEQ. ID. NO: 9.

In one instance, provided herein is an isolated antigen binding protein that specifically binds MrkA, wherein the antigen binding protein comprises a heavy chain variable region (VH) at least 95%, 96%, 97%, 98% or 99% identical to SEQ ID NO: 13 and/or a light chain variable region (VL) at least 95%0, 96%, 97%, 98% or 99% identical to SEQ ID NO: 15. In one instance, the antigen binding protein thereof comprises a VH comprising SEQ ID NO: 13 and a VL comprising SEQ ID NO: 15.

In one instance, provided herein is an isolated antigen binding protein that specifically binds to MrkA comprising a VH comprising SEQ ID NO: 13.

In one instance, provided herein is an isolated antigen binding protein that specifically binds to MrkA comprising a VL comprising SEQ ID NO: 15.

In one instance, provided herein is an isolated antigen binding protein that specifically binds MrkA comprising a set of Complementarity-Determining Regions (CDRs): HCDR1, HCDR2, HCDR3, LCDR1, LCDR2, and LCDR3 wherein: HCDR1 has the amino acid sequence of SEQ. ID. NO: 4; HCDR2 has the amino acid sequence of SEQ. ID. NO: 5; HCDR3 has the amino acid sequence of SEQ. ID. NO: 6; LCDR1 has the amino acid sequence of SEQ. ID. NO: 10; LCDR2 has the amino acid sequence of SEQ. ID. NO: 11; and LCDR3 has the amino acid sequence of SEQ. ID. NO: 12.

In one instance, provided herein is an isolated antigen binding protein that specifically binds MrkA, wherein said antigen binding protein comprises a heavy chain variable region (VH) at least 95%, 96%, 97%, 98%, or 99% identical to SEQ ID NO: 14 and/or a light chain variable region (VL) at least 95%, 96%, 97%, 98%, or 99% identical to SEQ ID NO: 16. In one instance, the antigen binding protein comprises a VH comprising SEQ ID NO: 14 and a VL comprising SEQ ID NO: 16.

In one instance, provided herein is an isolated antigen binding protein that specifically binds to MrkA comprising a VH comprising SEQ ID NO: 14.

In one instance, provided herein is an isolated antigen binding protein that specifically binds to MrkA comprising a VL comprising SEQ ID NO: 16.

In one instance, the antigen binding protein binds to an epitope in amino acids 1-40 and 171-202 of SEQ ID NO: 17. In one instance, the antigen binding protein specifically binds to MrkA (SEQ ID NO: 17), but does not bind to either SEQ ID NO:26 (MrkA lacking amino acids 1-40 of SEQ ID NO:17) or SEQ ID NO:27 (MrkA lacking amino acids 171-202 of SEQ ID NO:17).

In one instance, provided herein is an isolated antigen binding protein that specifically binds to MrkA, wherein the antigen binding protein binds to an epitope in amino acids 1-40 and 171-202 of SEQ ID NO:17.

In one instance, provided herein is an isolated antigen binding protein that specifically binds to MrkA (SEQ ID NO: 17), but does not bind to either SEQ ID NO:26 and/or SEQ ID NO:27.

In one instance, provided herein is an isolated antigen binding protein that specifically binds to MrkA comprising a set of Complementarity-Determining Regions (CDRs): HCDR1, HCDR2, HCDR3, LCDR1, LCDR2, and LCDR3 selected from the group consisting of: (i) SEQ ID NOs: 29-31 and 41-43, respectively; (ii) SEQ ID NOs: 32-34 and 44-46, respectively; (iii) SEQ ID NOs: 35-37 and 47-49, respectively; and (iv) SEQ ID NOs: 38-40 and 50-52, respectively.

In one instance, provided herein is an isolated antigen binding protein that specifically binds to MrkA, wherein said antigen binding protein comprises a heavy chain variable region (VH) at least 95%, 96%, 97%, 98%, or 99% identical to SEQ ID NO:53, 54, 55, or 56 and/or a light chain variable region (VL) at least 95%, 96%, 97%, 98%, or 99% identical to SEQ ID NO:57, 58, 59, or 60. In one instance, the antigen binding protein comprises a VH comprising SEQ ID NO:53, 54, 55, or 56 and a VL comprising SEQ ID NO:57, 58, 59, or 60.

In one instance, provided herein is an isolated antigen binding protein that specifically binds to MrkA comprising a VH comprising SEQ ID NO:53, 54, 55, or 56.

In one instance, provided herein is an isolated antigen binding protein that specifically binds to MrkA comprising a VL comprising SEQ ID NO:57, 58, 59, or 60. In one instance, provided herein is an isolated antigen binding protein that specifically binds to the same MrkA epitope as an antibody or antigen-binding fragment thereof selected from the group consisting of: (a) an antibody or antigen-binding fragment thereof comprising a heavy chain variable region (VH) comprising SEQ ID NO: 13 and a light chain variable region (VL) comprising SEQ ID NO: 15; (b) an antibody or antigen-binding fragment thereof comprising a heavy chain variable region (VH) comprising SEQ ID NO: 14 and a light chain variable region (VL) comprising SEQ ID NO: 16; (c) an antibody or antigen-binding fragment thereof comprising a heavy chain variable region (VH) comprising SEQ ID NO:53 and light chain variable region (VL) comprising SEQ ID NO:57; (d) an antibody or antigen-binding fragment thereof comprising a heavy chain variable region (VH) comprising SEQ ID NO:54 and light chain variable region (VL) comprising SEQ ID NO:58; (e) an antibody or antigen-binding fragment thereof comprising a heavy chain variable region (VH) comprising SEQ ID NO:55 and light chain variable region (VL) comprising SEQ ID NO:59; and (f) an antibody or antigen-binding fragment thereof comprising a heavy chain variable region (VH) comprising SEQ ID NO:56 and light chain variable region (VL) comprising SEQ ID NO:60. In one instance, provided herein is an isolated antigen binding protein that competitively inhibits binding of a reference antibody to MrkA, wherein said reference antibody is selected from the group consisting of: (a) an antibody or antigen-binding fragment thereof comprising a heavy chain variable region (VH) comprising SEQ ID NO: 13 and a light chain variable region (VL) comprising SEQ ID NO: 15; (b) an antibody or antigen-binding fragment thereof comprising a heavy chain variable region (VH) comprising SEQ ID NO: 14 and a light chain variable region (VL) comprising SEQ ID NO: 16; (c) an antibody or antigen-binding fragment thereof comprising a heavy chain variable region (VH) comprising SEQ ID NO:53 and light chain variable region (VL) comprising SEQ ID NO:57; (d) an antibody or antigen-binding fragment thereof comprising a heavy chain variable region (VH) comprising SEQ ID NO:54 and light chain variable region (VL) comprising SEQ ID NO:58; (e) an antibody or antigen-binding fragment thereof comprising a heavy chain variable region (VH) comprising SEQ ID NO:55 and light chain variable region (VL) comprising SEQ ID NO:59; and (f) an antibody or antigen-binding fragment thereof comprising a heavy chain variable region (VH) comprising SEQ ID NO:56 and light chain variable region (VL) comprising SEQ ID NO:60.

In one instance, the antigen binding protein or antigen-binding fragment thereof binds oligomeric MrkA.

In one instance, provided herein is an isolated antigen binding protein that specifically binds to oligomeric MrkA, but does not bind to monomeric MrkA.

In one instance, the antigen binding protein is murine, non-human, humanized, chimeric, resurfaced, or human.

In one instance, the antigen binding protein is an antibody. In some embodiments, the antigen binding protein is a monoclonal antibody, a recombinant antibody, a human antibody, a humanized antibody, a chimeric antibody, a bi-specific antibody, a multi-specific antibody, or an antigen binding fragment thereof.

In some embodiments, the antigen binding protein is an antigen binding fragment of an antibody. In one instance, the antigen binding protein comprises a Fab, Fab′, F(ab′)2, Fd, single chain Fv or scFv, disulfide linked Fv, V-NAR domain, IgNar, intrabody, IgGΔCH2, minibody, F(ab′)3, tetrabody, triabody, diabody, single-domain antibody, DVD-Ig, mAb2, (scFv)2, or scFv-Fc. In one instance, the antigen binding protein comprises a Fab, Fab′, F(ab′)2, single chain Fv or scFv, disulfide linked Fv, intrabody, IgGΔCH2, minibody, F(ab′)3, tetrabody, triabody, diabody, DVD-Ig, Fcab, mAb2, (scFv)2, or scFv-Fc.

In one instance, the antigen binding protein binds to MrkA with a Kd of about 1.0 nM to about 10 nM. In one instance, the antigen binding protein binds to MrkA with a Kd of 1.0 nM or less. In one instance, the binding affinity is measured by flow cytometry, Biacore, KinExa, radioimmunoassay, or bio-layer interferometry (BLI).

In one instance, the antigen binding protein a) binds to at least two Klebsiella pneumoniae (K. pneumoniae) serotypes; b) induces opsonophagocytic killing (OPK) of K. pneumoniae or c) binds to at least two K. pneumoniae serotypes and induces OPK of K. pneumonia.

In one instance, the antigen binding protein (including, e.g., an anti-MrkA antibody or antigen binding fragment thereof) inhibits or reduces Klebsiella biofilm formation. In some aspects, the antigen binding protein (including, e.g., an anti-MrkA antibody or antigen binding fragment thereof) inhibits or reduces Kp43816 biofilm formation.

In one instance, the antigen binding protein (including, e.g., an anti-MrkA antibody or antigen binding fragment thereof) inhibits or reduces Klebsiella cell attachment. In some aspects, the antigen binding protein (including, e.g., an anti-MrkA antibody or antigen binding fragment thereof) inhibits or reduces Klebsiella (including, e.g., Kp43816) cell attachment to a human cell. In some aspects, the antigen binding protein (including. e.g., an anti-MrkA antibody or antigen binding fragment thereof) inhibits or reduces Klebsiella (including, e.g., Kp43816) cell attachment to human epithelial cells. In some aspects, the antigen binding protein (including, e.g., an anti-MrkA antibody or antigen binding fragment thereof) inhibits or reduces Klebsiella (including, e.g., Kp43816) cell attachment to pulmonary epithelial cells. In some aspects, the antigen binding protein (including, e.g., an anti-MrkA antibody or antigen binding fragment thereof) inhibits or reduces Klebsiella (including, e.g., Kp43816) cell attachment to A549 cells.

In one instance, the antigen binding protein comprises a heavy chain immunoglobulin constant domain selected from the group consisting of: (a) an IgA constant domain; (b) an IgD constant domain; (c) an IgE constant domain; (d) an IgG1 constant domain; (e) an IgG2 constant domain; (f) an IgG3 constant domain; (g) an IgG4 constant domain; and (h) an IgM constant domain. In one instance, the antigen binding protein comprises a light chain immunoglobulin constant domain selected from the group consisting of: (a) an Ig kappa constant domain; and (b) an Ig lambda constant domain. In one instance, the antigen binding protein comprises a human IgG1 constant domain and a human lambda constant domain.

In one instance, the antigen binding protein comprises an IgG1 constant domain.

In one instance, the antigen binding protein comprises an IgG1/IgG3 chimeric constant domain.

In one instance, provided herein is a hybridoma producing the antigen binding protein (including, e.g., an anti-MrkA antibody or antigen binding fragment thereof).

In one instance, provided herein is an isolated host cell producing the antigen binding protein (including, e.g., an anti-MrkA antibody or antigen binding fragment thereof).

In one instance, provided herein is a method of making the antigen binding protein (including, e.g., an anti-MrkA antibody or antigen binding fragment thereof) comprising (a) culturing a host cell expressing said antigen binding protein; and (b) isolating said antigen binding protein thereof from said cultured host cell. In one instance, provided herein is an antigen binding protein (including, e.g., an anti-MrkA antibody or antigen binding fragment thereof) produced using the method.

The present disclosure also provides a pharmaceutical composition comprising the antigen binding protein (including, e.g., an anti-MrkA antibody or antigen binding fragment thereof) and a pharmaceutically acceptable excipient. In one instance, the pharmaceutically acceptable excipient is a preservative, stabilizer, or antioxidant. In one instance, the pharmaceutical composition is for use as a medicament.

In one instance, the antigen binding protein or the pharmaceutical composition further comprises a labeling group or an effector group. In one instance, the labeling group is selected from the group consisting of: isotopic labels, magnetic labels, redox active moieties, optical dyes, biotinylated groups, fluorescent moieties such as biotin signaling peptides, Green Fluorescent Proteins (GFPs), blue fluorescent proteins (BFPs), can fluorescent proteins (CFPs), and yellow fluorescent proteins (YFPs), and polypeptide epitopes recognized by a secondary reporter such as histidine peptide (his), hemagglutinin (HA), gold binding peptide, and Flag. In one instance, the effector group is selected from the group consisting of a radioisotope, radionuclide, a toxin, a therapeutic and a chemotherapeutic agent.

In one instance, provided herein is the use of an antigen binding protein (including, e.g., an anti-MrkA antibody or antigen binding fragment thereof) or pharmaceutical composition provided herein for treating or preventing a condition associated with a Klebsiella infection.

The present disclosure also provides a method for treating, preventing, or ameliorating a condition associated with a Klebsiella infection in a subject in need thereof comprising administering to the subject an effective amount of an antigen binding protein (including, e.g., an anti-MrkA antibody or antigen binding fragment thereof) or pharmaceutical composition provided herein.

In one instance, provided herein is a method for inhibiting the growth of Klebsiella in a subject comprising administering to a subject in need thereof an antigen binding protein (including, e.g., an anti-MrkA antibody or antigen binding fragment thereof) or pharmaceutical composition provided herein.

In one instance, provided herein is a method for treating, preventing, or ameliorating a condition associated with a Klebsiella infection in a subject in need thereof comprising administering to said subject an effective amount of an anti-MrkA antibody or an antigen binding fragment thereof. In some embodiments, the condition is selected from the group consisting of pneumonia, urinary tract infection, septicemia, neonatal septicemia, diarrhea, soft tissue infection, infection following an organ transplant, surgery infection, wound infection, lung infection, pyogenic liver abscesses (PLA), endophthalmitis, meningitis, necrotizing meningitis, ankylosing spondylitis, and spondyloarthropathies. In one instance, the condition is a nosocomial infection. In one instance, the Klebsiella is K. pneumonia. K. oxytoca, K. planticola and/or K. granulomatis. In one instance, the Klebsiella is resistant to cephalosporin, aminoglycoside, quinolone, and/or carbapenem. In one instance, the method further comprises administering an antibiotic. In one instance, the antibiotic is a carbapanem or colistin.

In one instance, provided herein is a method for inhibiting the growth of Klebsiella in a subject comprising administering to a subject in need thereof an effective amount of an anti-MrkA antibody or an antigen binding fragment thereof. In some embodiments, the anti-MrkA antibody or antigen binding fragment thereof specifically binds to K. pneumonia, K. oxytoca, K. planticola and/or K. granulomatis MrkA. In one instance, the anti-MrkA antibody or antigen binding fragment thereof specifically bins to K. pneumonia MrkA

The present disclosure also provides an isolated nucleic acid molecule encoding an antigen binding protein provided herein.

In one instance, provided herein is an isolated nucleic acid molecule encoding a heavy chain variable region (VH) sequence selected from the group consisting of SEQ ID NOs: 13, 14, 53, 54, 55, and 56. In one instance, provided herein is an isolated nucleic acid molecule encoding a light chain variable region (VL) sequence selected from the group consisting of SEQ ID NOs:15, 16, 57, 58, 59, and 60.

In one instance, the nucleic acid molecule is operably linked to a control sequence. In one instance, provided herein is a vector comprising a nucleic acid molecule provided herein. In one instance, provided herein is a host cell transformed with a nucleic acid molecule provided herein or a vector provided herein.

In one instance, provided herein is a host cell transformed with a nucleic acid encoding a heavy chain variable region (VH) sequence selected from the group consisting of SEQ ID NOs: 13, 14, 53, 54, 55, and 56 and a nucleic acid molecule encoding a VL sequence selected from the group consisting of SEQ ID NOs: 15, 16, 57, 58, 59, and 60.

In one instance, the host cell is a mammalian host cell. In one instance, the host cell is a NS0 murine myeloma cell, a PER.C6® human cell, or a Chinese hamster ovary (CHO) cells.

The present disclosure also provides a pharmaceutical composition comprising MrkA, an immunogenic fragment thereof, or a polynucleotide encoding MrkA or an immunogenic fragment thereof. In one instance, the disclosure provides a vaccine comprising MrkA, an immunogenic fragment thereof, or a polynucleotide encoding MrkA or an immunogenic fragment thereof. In some embodiments, the pharmaceutical composition or vaccine comprises an immunologically effective amount of the MrkA, immunogenic fragment thereof, or polynucleotide encoding MrkA or an immunogenic fragment thereof. In one instance, the pharmaceutical composition or vaccine comprises an adjuvant. In one instance, the MrkA or immunogenic fragment thereof of the pharmaceutical composition or vaccine is monomeric. In one instance, the MrkA or immunogenic fragment thereof of the pharmaceutical composition or vaccine is oligomeric. In one instance, the MrkA is K. pneumonia MrkA.

In some embodiments, the MrkA or immunogenic fragment thereof comprises a sequence at least 75%, at least 80%, at least 85%, at least 90%, at least 95%, at least 96%, at least 97%, at least 98%, or at least 99% identical to the sequence set forth in SEQ ID NO: 17 or wherein the polynucleotide encoding MrkA or an immunogenic fragment thereof encodes a sequence at least 75%, at least 80%, at least 85%, at least 90%, at least 95%, at least 96%, at least 97%, at least 98%, or at least 99% identical to the sequence set forth in SEQ ID NO: 17. In one instance, the MrkA or immunogenic fragment thereof comprises the sequence set forth in SEQ ID NO: 17 or wherein the polynucleotide encoding MrkA or an immunogenic fragment thereof encodes the sequence set forth in SEQ ID NO: 17.

The present disclosure also provides a method of inducing an immune response against Klebsiella in a subject comprising administering to the subject a pharmaceutical composition, a MrkA or immunogenic fragment thereof, or vaccine provided herein. In one instance, the immune response comprises an antibody response. In one instance, the immune response comprises a cell-mediated immune response. In one instance, the immune response comprises a cell-mediated immune response and an antibody response. In one instance, the immune response is a mucosal immune response. In one instance, the immune response is a protective immune response.

In addition, provided herein is a method of vaccinating a subject against Klebsiella comprising administering to a subject the pharmaceutical composition, MrkA or immunogenic fragment thereof, or vaccine provided herein. In one instance, provided herein is a method for treating, preventing, or reducing the incidence of a condition associated with a Klebsiella infection in a subject in need thereof comprising administering to said subject MrkA, an immunogenic fragment thereof, or a polynucleotide encoding MrkA or an immunogenic fragment thereof. In one instance, provided herein is a method for inhibiting the growth of Klebsiella in a subject comprising administering to a subject in need thereof MrkA, an immunogenic fragment thereof, or a polynucleotide encoding MrkA or an immunogenic fragment thereof. In one instance of the methods provided herein, the Klebsiella is K. pneumonia, K. oxytoca, K. planticola and/or K. granulomatis. In one instance, the Klebsiella is K. pneumonia. In one instance of the methods provided herein, the MrkA or immunogenic fragment thereof is monomeric. In one instance of the methods provided herein, the MrkA or immunogenic fragment thereof is oligomeric. In one instance of the methods provided herein, the MrkA is K. pneumonia MrkA.

### BRIEF DESCRIPTION OF THE DRAWINGS/FIGURES

FIGS. 1A-F depict the K. pneumoniae binding and potent OPK activity of monoclonal antibodies (mAbs) isolated through phage and hybridoma platforms. A: Antibody binding to Kp29011 in a whole cell ELISA assay: two hybridoma clones (88D10 and 89E10) and two phage antibodies (Kp3 and Kp16) bind to K. pneumoniae strain 29011 in ELISA assays as described in Example 2. As expected, control antibody hIgG control did not bind to K. pneumoniae strain 29011. B: Antibodies induce opsonophagocytic killing (OPK) of K. pneumoniae. Phage (Kp3 and Kp16) and hybridoma (88D10 and 89E10) derived antibodies were incubated with baby rabbit serum, HL60, and K. pneumoniae strain 29011.lux. Bacterial killing was calculated in comparison with control lacking antibody. C: Phage antibodies (Kp3 and Kp16) compete for binding to K. pneumoniae. One g/ml of biotin-labeled Kp3 was mixed with increasing amount of unlabeled phage and control antibodies as indicated and tested for its binding to K. pneumoniae strain 29011. Streptavidin-HRP was used as the detecting agent. Kp3 and Kp16 both prevented binding of biotin-labeled Kp3 to K. pneumoniae strain 29011. D: Phage (Kp3 and Kp16) and hybridoma antibodies (88D10) compete in binding to K. pneumoniae. One g/ml of hybridoma clone 88D10 was mixed with increasing amount of phage and control antibodies (hIgG) and tested for its binding to K. pneumoniae strain 29011. Anti-mouse-IgG-HRP was used as the detecting agent. The reduction in ELISA signal was expressed as a percentage of inhibition. Kp3 and Kp16 both prevented binding of 88D10 to K. pneumoniae strain 2901. E. Phage (Kp3 and Kp16) and hybridoma (21G10, 22B12, 88D10 and 89E10) antibodies bind to K. pneumoniae strains with various serotypes. “+” indicates binding. F. Anti-MrkA mAb Kp3 displays potent OPK activity against K. pneumoniae of different serotypes.

FIGS. 2A-D depict the results of experiments identifying MrkA as the antigen bound by K. pneumoniae specific antibodies generated herein. A: Confocal microscopy image showing Kp3 antibody binding to the surface of K. pneumoniae. B: Immunoprecipitation by Kp3, 88D10, and an isotype control antibody from cell lysates from non-reactive (1899) and reactive (43816DM) K. pneumoniae strains. The numbered bands (1 to 4) corresponding to immunoprecipitated polypeptides were subjected to LC-MS analysis. C: Western blot analysis of the immunoprecipitation products. The lanes in FIGS. 2B and C were as follows: Lane 1—pre-stained molecular weight marker; Lane 2—cell lysate from Kp3 nonreactive strain 1899; Lane 3—cell lysate from Kp3 reactive strain 43816DM; Lane 4 —1899 lysate subjected to immunoprecipitation by isotype control; Lane 5-1899 lysate subjected to immunoprecipitation by Kp3; Lane 6-1899 lysate subjected to immunoprecipitation by 88D10; Lane 7-43816DM lysate subjected to immunoprecipitation by isotype control; Lane 8-43816DM lysate subjected to immunoprecipitation by Kp3; and Lane 9-43816DM lysate subjected to immunoprecipitation by 88D10. D: LC-MS result of gel band number 3 from FIG. 2B. Peptides identified through mass spectrometry are in bold and underlined in the context of the K. pneumoniae strain MGH78578 MrkA sequence (SEQ ID NO: 17).

FIGS. 3A-B show MrkA is the common antigen bound by K. pneumoniae specific antibodies generated herein. A: Recombinant expression of MrkA by Western blot analysis using anti-his tag (left panel) and Kp3 (right panel) antibodies. Lane 1: host cell only; Lane 2: host cell transformed with empty vector; Lane 3: host cell transformed with expression vector carrying his-tagged MrkA ORF; and Lane 4: lysate prepared from strain 43816DM. These results show that Kp3 binds to recombinant MrkA. B: In vitro transcription and translation of MrkA and Western blot analysis using Kp3 (left panel) anti-Myc tag (right panel) antibodies. Samples 1: positive bacterial cell lysate; 2: negative cell lysate; 3: in vitro expressed MrkA without signal peptide/with disulfide bond enhancer; 4: with signal peptide/with disulfide bond enhancer; 5: without either signal peptide or disulfide bond enhancer; 6: with signal peptide but no disulfide bond enhancer; and 7: In vitro expression system negative control without MrkA ORF. These results show that Kp3 binds to in vitro translated MrkA. Numbers on the left sides of both FIGS. 3A and 3B are protein molecular weights in kDa.

FIGS. 4A-D depict the protective activity of Kp3 mAb in various in vivo models. A and B: Kp3 reduces organ burden in intranasal lung infection model against Kp29011 (O1:K2) and Kp9178 (O3:K38), respectively. An irrelevant human IgG1 antibody (hIgG1) and rabbit polyclonal antibody against Kp43816 (Rab IgG) were used as controls. All antibodies were used at a dose of 15 mg/kg. These results show that anti-MrkA antibody Kp3 reduced organ burden when administered prior to bacterial challenge. C: Kp3 enhanced survival in a lethal bacterial pneumonia model using Kp43816 (O1:K2). An irrelevant human IgG1 (hIgG1) antibody was used as a control. Both antibodies were used at a dose of 15 mg/l kg. D: Kp3 significantly enhanced survival in a lethal bacterial pneumonia model using Kp985048, a multi-drug resistance (MDR) strain. An irrelevant human IgG1 (hlgG1) antibody was used as a control. Both antibodies were used at a dose of 5 mg/kg. These results show that anti-MrkA antibody Kp3 enhances survival when administered 24 hours before bacterial challenge.

FIG. 5 depicts MrkA conservation among the enterobactereaceae family members. Conserved residues are displayed at the top, and divergent residues are marked with a box. MrkA is conserved among the majority of enterobactereace family members.

FIG. 6 depicts the results of MrkA binding assays. Full length MrkA (“MrkA-WT”; SEQ ID NO: 17), MrkA with a 40 amino acid N-terminal deletion (“MrkA-N-dlt”; i.e., amino acids 41-202 of SEQ ID NO:17 (i.e., SEQ ID NO:26)), MrkA with a 32 amino acid C-terminal deletion (“MrkA-C-dlt”; i.e., amino acids 1-170 of SEQ ID NO:17 (i.e., SEQ ID NO:27)), MrkA with both the N and C terminal deletions (“MrkA-N/C-dlt”; i.e., amino acids 41-170 of SEQ ID NO:17 (i.e., SEQ ID NO:28)), and an empty vector (“Top10 cont”) were expressed in cells. Cell lysates was coated directly onto ELISA plates and assayed for binding with Kp3 and a control MrkA antibody. Human IgG1 also served as a control. Kp3 only detected full length MrkA, whereas the control antibody detected full length MrkA as well as MrkA with N terminal deletion. These results show that Kp3 recognizes a conformational epitope.

FIG. 7 depicts purification of monomeric and oligomeric MrkA. Fractions of monomeric and oligomeric MrkA were expressed, purified, and analyzed by SDS-PAGE gel under reducing and non-reducing conditions and visualized with blue stain. M: molecular weight marker. Lanes 1 and 4 contain monomeric MrkA from pool 1. Lanes 2 and 5 contain monomeric MrkA from pool 2. Lanes 3 and 6 contain oligomeric MrkA.

FIGS. 8A-B shows that MrkA vaccination reduces lung burden. C57/bl6 mice immunized with monomeric or oligomeric MrkA were challenged with Kp29011 (O1:K2) intra-nasally. The presence of bacteria in lung and liver were analyzed 24 hours post infection. Monomeric MrkA significantly reduced bacteria in the lung (FIG. 8A), and oligomeric MrkA significantly reduced bacteria in both the lung and liver (FIG. 8B). (*) indicates Student's t test p value <0.05.

FIG. 9 shows that Kp3 inhibits Klebsiella biofilm formation. Kp43816 was added to Falcon plastic plates in the presence of the anti-MrkA antibody Kp3 (closed triangles), or hIgG1 (isotype control antibodies, open triangles, “R347”). The inhibition of biofilm formation was graphed. (**) indicates Student's t test p value <0.01 for Kp3 values relative to isotype control.

FIG. 10 shows that Kp3 inhibits Klebsiella binding to epithelial cells. Kp43816 was added to A549 cells (2×10⁵/well) in the presence of the anti-MrkA antibody Kp3 (closed triangles), or higGI (open triangles, “R347”). Samples were run in duplicate; graph is representative of 3 separate experiments. (*) indicates Student's t test p value <0.05 for Kp3 values relative to isotype control. Where error bars cannot be seen they are smaller than the symbol width.

FIG. 11 shows the phage panning output screening cascade described in Example 10. More than 4000 colonies were picked for high throughput screening after phage panning, scFv.Fc conversion, and transformation. Four clones including clones 1, 4, 5, and 6 were selected for further characterization.

FIG. 12 shows a schematic representation of a four-component homogeneous time resolved FRET (HTRF) used for screening for MrkA binders. Component A, which is Streptavidin-Eu(K) cryptate and serves as the energy donor, is brought into close proximity of component D, which is anti-huFc-alexa fluor 647 and serves as the energy acceptor by the interaction between components B and C. B is the biotin-labeled MrkA, and C is a scFv-Fc specific for MrkA.

FIGS. 13A-B shows binding assays using anti-MrkA antibodies. MrkA protein was either coated directly onto the ELISA plate (B) or captured by streptavidin after biotinylation (A). The MrkA protein was recognized differently by anti-MrkA antibodies in these different antigen-presentation formats.

FIG. 14 shows that anti-MrkA antibodies bind preferably to the oligomeric MrkA prepared directly from a KP strain (K) as compared to the recombinant MrkA expressed in E. coli (E) in a Western blot analysis. Clone 1 is the only antibody capable of detecting the monomeric MrkA from KP (indicated by an arrow).

FIGS. 15A-C shows the result of epitope binding assays. Epitope binning was performed against three test articles: KP3 (A), clone 4 (B), and clone 5 (C).

FIGS. 16A-B demonstrates that OPK activity is important for in vivo protective activities. KP3-TM mutation was generated and tested in both an in vitro OPK assay (A) and an in viov challenge assay (B). Significant reduction was seen in the OPK assay, and a trend towards significance was seen in the in vivo challenge assay.

FIGS. 17A-C shows serotype-independent binding to KP strains by anti-MrkA antibodies. A flow cytometry experiment was used to gauge the binding of four anti-MrkA antibodies against three WT KP strains of different serotypes, 29011 (O1:K2) (A); 961842 (O2) (B): and 985048 (O4) (C). R347 is a human IgG isotype control.

FIGS. 18A-B shows serotype-independent OPK activities by anti-MrkA antibodies. Two strains of LPS serotvpes O1 (A) and O2 (B) were used in the OPK assay. The anti-MrkA antibodies clone 1, clone 4, clone 5, and clone 6 displayed comparable OPK activities to that of KP3. R347 is a human IgG isotype control.

FIG. 19 shows the results of a prophylactic in vivo challenge model. Antibodies were given 24 hours prior to KP challenge.

FIG. 20 shows the results of a therapeutic in vivo challenge model. Antibodies were given one hour after KP challenge.

FIG. 21 shows that individual antibodies are as effective as antibody combinations in the therapeutic model. KP3 was combined with either clone 1 or clone 5 in equal amount as indicated and tested in a therapeutic model.

### DETAILED DESCRIPTION OF THE INVENTION

The present disclosure provides isolated binding proteins, including antibodies or antigen binding fragments thereof, which bind to MrkA. Related polynucleotides, vectors, host cells, and pharmaceutical compositions comprising the MrkA binding proteins, including antibodies or antigen binding fragments thereof, are also provided. Also provided are methods of making and using the MrkA binding proteins, including antibodies or antigen binding fragments, disclosed herein. The present disclosure also provides methods of preventing and/or treating a condition associated with a Klebsiella infection by administering the MrkA binding proteins, including antibodies or antigen binding fragments, disclosed herein.

In order that the present disclosure can be more readily understood, certain terms are first defined. Additional definitions are set forth throughout the detailed description.

### I. Definitions

The terms “a,” “an,” and “the” include plural referents unless the context clearly dictates otherwise. For example, “an antigen binding protein” is understood to represent one or more antigen binding proteins. The terms “a” (or “an”), as well as the terms “one or more,” and “at least one” can be used interchangeably herein. Furthermore. “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C: A and B; B and C; A (alone); B (alone); and C (alone).

The term “comprise” is generally used in the sense of include, that is to say permitting the presence of one or more features or components. Wherever aspects are described herein with the language “comprising,” otherwise analogous aspects described in terms of “consisting of,” and/or “consisting essentially of” are also provided.

The term “about” as used in connection with a numerical value throughout the specification and the claims denotes an interval of accuracy, familiar and acceptable to a person skilled in the art. In general, such interval of accuracy is ±10%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme Intemational de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The term “antigen binding protein” refers to a molecule comprised of one or more polypeptides that recognizes and specifically binds to a target, e.g., MrkA, such as an anti-MrkA antibody or antigen-binding fragment thereof.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, multispecific antibodies such as bispecific antibodies generated from at least two intact antibodies, chimeric antibodies, humanized antibodies, human antibodies, fusion proteins comprising an antibody, and any other modified immunoglobulin molecule so long as the antibodies exhibit the desired biological activity. An antibody can be any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” or “antibody fragment thereof” refers to a portion of an intact antibody. An “antigen-binding fragment” or “antigen-binding fragment thereof” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, scFvs, and single chain antibodies.

It is possible to take monoclonal and other antibodies or fragments thereof and use techniques of recombinant DNA technology to produce other antibodies or chimeric molecules or fragments thereof that retain the specificity of the original antibody or fragment. Such techniques can involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A, or EP-A-239400, and a large body of subsequent literature. A hybridoma or other cell producing an antibody can be subject to genetic mutation or other changes, which may or may not alter the binding specificity of antibodies or fragments thereof produced.

Further techniques available in the art of antibody engineering have made it possible to isolate human and humanized antibodies or fragments thereof. For example, human hybridomas can be made as described by Kontermann and Sefan. Antibody Engineering, Springer Laboratory Manuals (2001). Phage display, another established technique for generating antigen binding proteins has been described in detail in many publications such as Kontermann and Sefan. Antibody Engineering, Springer Laboratory Manuals (2001) and WO92/01047. Transgenic mice in which the mouse antibody genes are inactivated and functionally replaced with human antibody genes while leaving intact other components of the mouse immune system, can be used for isolating human antibodies to human antigens.

Synthetic antibody molecules or fragments thereof can be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

It has been shown that fragments of a whole antibody can perform the function of binding antigens. Examples of binding fragments are (i) the Fab fragment consisting of VL, VH, CL, and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989), McCafferty et al (1990) Nature, 348, 552-554) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA. 85, 5879-5883, 1988); (viii) bispecific single chain Fv dimers (PCT/US92/09965) and (ix) “diabodies,” multivalent or multispecific fragments constructed by gene fusion (WO94/13804; P. Holliger et al, Proc. Natl. Acad. Sci. USA 90 6444-6448, 1993). Fv, scFv or diabody molecules may be stabilized by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects ofanti-idiotypic reaction. Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against MrkA, then a library can be made where the other arm is varied and an antibody of appropriate specificity selected. Bispecific whole antibodies may be made by knobs-into-holes engineering (J. B. B. Ridgeway et al, Protein Eng., 9, 616-621, 1996). Immunoglobulin-like domain-based technologies that have created multispecific and/or multivalent molecules include dAbs, TandAbs, nanobodies, BiTEs, SMIPs, DNLs, Affibodies, Fynomers, Kunitz Domains, Albu-dabs, DARTs, DVD-IG, Covx-bodies, peptibodies, scFv-Igs, SVD-Igs, dAb-Igs, Knobs-in-Holes, DuoBodies™ and triomAbs. Bispecific bivalent antibodies, and methods of making them, are described, for instance in U.S. Pat. Nos. 5,731,168; 5,807,706; 5,821,333; and U.S. Patent Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148: 1547-1553 (1992).

The phrase “effector function” refers to the activities of antibodies that result from the interactions of their Fc components with Fc receptors or components of complement. These activities include, for example, antibody-dependent cell-mediated cytotoxicity (ADCC), complement-dependent cytotoxicity (CDC), and antibody-dependent cell phagocytosis (ADCP). Thus an antigen binding protein (e.g., an antibody or antigen binding fragment thereof) with altered effector function refers to an antigen binding protein (e.g., an antibody or antigen binding fragment thereof) that contains an alteration in an Fc region (e.g., amino acid substitution, deletion, or addition or change in oligosaccharide) that changes the activity of at least one effector function (e.g., ADCC, CDC, and/or ADCP). An antigen binding protein (e.g., an antibody or antigen binding fragment thereof) with improved effector function refers to an antigen binding protein (e.g., an antibody or antigen binding fragment thereof) that contains an alteration in an Fc region (e.g., amino acid substitution, deletion, or addition or change in oligosaccharide) that increases the activity of at least one effector function (e.g., ADCC, CDC, and/or ADCP).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the antigen binding protein carrying the antigen binding domain will be able to bind to the various antigens carrying the epitope.

By “specifically binds” it is generally meant that an antigen binding protein including an antibody or antigen binding fragment thereof binds to an epitope via its antigen binding domain, and that the binding entails some complementarity between the antigen binding domain and the epitope. According to this definition, an antibody is said to “specifically bind” to an epitope when it binds to that epitope via its antigen binding domain more readily than it would bind to a random, unrelated epitope.

“Affinity” is a measure of the intrinsic binding strength of a ligand binding reaction. For example, a measure of the strength of the antibody (Ab)-antigen (Ag) interaction is measured through the binding affinity, which may be quantified by the dissociation constant, k_{d}. The dissociation constant is the binding affinity constant and is given by:

K_{d}=[Ab][Ag]
[AbAg complex]
   Affinity may, for example, be measured using a BIAcore®, a KinExa affinity assay, flow cytometry, and/or radioimmunoassay.

“Potency” is a measure of pharmacological activity of a compound expressed in terms of the amount of the compound required to produce an effect of given intensity. It refers to the amount of the compound required to achieve a defined biological effect; the smaller the dose required, the more potent the drug. Potency of an antigen binding protein that binds MrkA may, for example, be determined using an OPK assay, as described herein.

“Opsonophagocytic killing” or “OPK” refers to the death of a cell, e.g., a Klebsiella, that occurs as a result of phagocytosis by an immune cell. Assays that can be used to demonstrate OPK activity include the bio-luminescent OPK activity used in the Examples or by counting the bacterial colonies on Agar plates. Additional assays are provided, for example, in DiGiandomenico et al., J. Exp. Med. 209: 1273-87 (2012), which is incorporated herein by reference.

An antigen binding protein including an antibody or antigen binding fragment thereof is said to competitively inhibit binding of a reference antibody or antigen binding fragment thereof to a given epitope or “compete” with a reference antibody or antigen binding fragment if it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope or compete with a reference antibody or antigen binding fragment thereof by at least 90%, at least 80%, at least 70%, at least 60%, or at least 50%.

The term “compete” when used in the context of antigen binding proteins (e.g., neutralizing antigen binding proteins or neutralizing antibodies) means competition between antigen binding proteins as determined by an assay in which the antigen binding protein (e.g., antibody or immunologically functional fragment thereof) under test prevents or inhibits specific binding of a reference antigen binding protein (e.g., a ligand, or a reference antibody) to a common antigen (e.g., an MrkA protein or a fragment thereof). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 92:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see. e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein.

Competitive inhibition can be measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Usually, when a competing antigen binding protein is present in excess, it will inhibit specific binding of a reference antigen binding protein to a common antigen by at least 40%, 45%, 50%, 55%, 60%, 65%, 70% or 75%. In some instance, binding is inhibited by at least 80%, 85%, 90%, 91%, 92%, 93%, 94%, 95%, 96%, 97% 98%, 99% or more.

Antigen binding proteins, antibodies or antigen binding fragments thereof disclosed herein can be described or specified in terms of the epitope(s) or portion(s) of an antigen, e.g., a target polypeptide that they recognize or specifically bind. For example, the portion of MrkA that specifically interacts with the antigen binding domain of the antigen binding polypeptide or fragment thereof disclosed herein is an “epitope”. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. A conformational epitope can be composed of discontinuous sections of the antigen's amino acid sequence. A linear epitope is formed by a continuous sequence of amino acids from the antigen. Epitope determinants may include chemically active surface groupings of molecules such as amino acids, sugar side chains, phosphoryl or sulfonyl groups, and can have specific three dimensional structural characteristics, and/or specific charge characteristics. An epitope typically includes at least 3, 4, 5, 6, 3, 4, 5, 6, 7, 8, 9, 10, 11, 12, 13, 14, 15, 16, 17, 18, 19, 20, 25, 30, 35 amino acids in a unique spatial conformation. Epitopes can be determined using methods known in the art.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The term “isolated” refers to the state in which antigen binding proteins of the disclosure, or nucleic acid encoding such binding proteins, will generally be in accordance with the present disclosure. Isolated proteins and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practiced in vitro or in vivo. Proteins and nucleic acid may be formulated with diluents or adjuvants and still for practical purposes be isolated—for example the proteins will normally be mixed with gelatin or other carriers if used to coat microtitre plates for use in immunoassays, or will be mixed with pharmaceutically acceptable carriers or diluents when used in diagnosis or therapy. Antigen binding proteins may be glycosylated, either naturally or by systems of heterologous eukaryotic cells (e.g. CHO or NS0 (ECACC 85110503) cells), or they may be (for example if produced by expression in a prokaryotic cell) unglycosylated.

A polypeptide, antigen binding protein, antibody, polynucleotide, vector, cell, or composition which is “isolated” is a polypeptide, antigen binding protein, antibody, polynucleotide, vector, cell, or composition which is in a form not found in nature. Isolated polypeptides, antigen binding proteins, antibodies, polynucleotides, vectors, cells, or compositions include those which have been purified to a degree that they are no longer in a form in which they are found in nature. In some embodiments, an antigen binding protein, antibody, polynucleotide, vector, cell, or composition which is isolated is substantially pure.

A “recombinant” polypeptide, protein or antibody refers to a polypeptide or protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

Also included in the present disclosure are fragments, variants, or derivatives of polypeptides, and any combination thereof. The term “fragment” when referring to polypeptides and proteins of the present disclosure include any polypeptides or proteins which retain at least some of the properties of the reference polypeptide or protein. Fragments of polypeptides include proteolytic fragments, as well as deletion fragments.

The term “variant” as used herein refers to an antibody or polypeptide sequence that differs from that of a parent antibody or polypeptide sequence by virtue of at least one amino acid modification. Variants of antibodies or polypeptides of the present disclosure include fragments, and also antibodies or polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “derivatives” as applied to antibodies or polypeptides refers to antibodies or polypeptides which have been altered so as to exhibit additional features not found on the native polypeptide or protein. An example of a “derivative” antibody is a fusion or a conjugate with a second polypeptide or another molecule (e.g., a polymer such as PEG, a chromophore, or a fluorophore) or atom (e.g., a radioisotope).

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “host cell” refers to a cell or a population of cells harboring or capable of harboring a recombinant nucleic acid. Host cells can be prokaryotic cells (e.g., E. coli), or alternatively, the host cells can be eukaryotic, for example, fungal cells (e.g., yeast cells such as Saccharomyces cerivisiae, Pichia pastoris, or Schizosaccharomyces pombe), and various animal cells, such as insect cells (e.g., Sf-9) or mammalian cells (e.g., HEK293F, CHO, COS-7, NIH-3T3, a NS0 murine mveloma cell, a PER.C6® human cell, a Chinese hamster ovary (CHO) cell or a hybridoma).

The term “amino acid substitution” refers to replacing an amino acid residue present in a parent sequence with another amino acid residue. An amino acid can be substituted in a parent sequence, for example, via chemical peptide synthesis or through recombinant methods known in the art. Accordingly, references to a “substitution at position X” refer to the substitution of an amino acid present at position X with an alternative amino acid residue. In some embodiments, substitution patterns can be described according to the schema AXY, wherein A is the single letter code corresponding to the amino acid naturally present at position X, and Y is the substituting amino acid residue. In other aspects, substitution patterns can described according to the schema XY, wherein Y is the single letter code corresponding to the amino acid residue substituting the amino acid naturally present at position X.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala. Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala, Ser) or no side chain (e.g., Gly).

Other substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, omithine, or D-omithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

The term “amino acid insertion” refers to introducing a new amino acid residue between two amino acid residues present in the parent sequence. An amino acid can be inserted in a parent sequence, for example, via chemical peptide synthesis or through recombinant methods known in the art. Accordingly as used herein, the phrases “insertion between positions X and Y” or “insertion between Kabat positions X and Y,” wherein X and Y correspond to amino acid positions (e.g., a cysteine amino acid insertion between positions 239 and 240), refers to the insertion of an amino acid between the X and Y positions, and also to the insertion in a nucleic acid sequence of a codon encoding an amino acid between the codons encoding the amino acids at positions X and Y. Insertion patterns can be described according to the schema AXins, wherein A is the single letter code corresponding to the amino acid being inserted, and X is the position preceding the insertion.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tool/psa.

“Specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. The present disclosure is concerned with antigen-antibody type reactions.

The term “IgG” as used herein refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3.

The term “antigen binding domain” describes the part of an antibody molecule which comprises the area which specifically binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). An antigen binding domain may comprise an antibody light chain variable region (VL) and an antibody heavy chain variable region (VH).

The term “antigen binding protein fragment” or “antibody fragment” refers to a portion of an intact antigen binding protein or antibody and refers to the antigenic determining variable regions of an intact antigen binding protein or antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides. The term “humanized antibody” refers to an antibody derived from a non-human (e.g., murine) immunoglobulin, which has been engineered to contain minimal non-human (e.g., murine) sequences.

The term “chimeric antibody” refers to antibodies wherein the amino acid sequence of the immunoglobulin molecule is derived from two or more species. Typically, the variable region of both light and heavy chains corresponds to the variable region of antibodies derived from one species of a mammal (e.g., mouse, rat, rabbit, etc.) with the desired specificity, affinity, and capability while the constant regions are homologous to the sequences in antibodies derived from another (usually human) to avoid eliciting an immune response in that species.

The term “antibody binding site” refers to a region in the antigen (e.g., MrkA) comprising a continuous or discontinuous site (i.e., an epitope) to which a complementary antibody specifically binds. Thus, the antibody binding site can contain additional areas in the antigen which are beyond the epitope and which can determine properties such as binding affinity and/or stability, or affect properties such as antigen enzymatic activity or dimerization. Accordingly, even if two antibodies bind to the same epitope within an antigen, if the antibody molecules establish distinct intermolecular contacts with amino acids outside of the epitope, such antibodies are considered to bind to distinct antibody binding sites.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service. National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The IMGT (Lefranc, M.-P. et al. Dev. Comp. Immunol. 27: 55-77 (2003)) classification of CDRs can also be used.

The term “EU index as in Kabat” refers to the numbering system of the human IgG1 EU antibody described in Kabat et al., Sequences of Immunological Interest, 5th Ed. Public Health Service, National Institutes of Health. Bethesda, Md. (1991). All amino acid positions referenced in the present application refer to EU index positions. For example, both “L234” and “EU L234” refer to the amino acid leucine at position 234 according to the EU index as set forth in Kabat.

The terms “Fc domain,” “Fc Region,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. For example, an Fc domain contains the entire second constant domain CH2 (residues at EU positions 231-340 of human IgG1) and the third constant domain CH3 (residues at EU positions 341-447 of human IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358. Thus, a “wild type IgG Fc domain” or “WT IgG Fc domain” refers to any naturally occurring IgG Fc region (i.e., any allele). Myriad Fc mutants, Fc fragments, Fc variants, and Fc derivatives are described, e.g., in U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; U.S. Patent publication 2004/0002587; and PCT Publication Nos. WO 99/058572, WO 2011/069164 and WO 2012/006635.

The sequences of the heavy chains of human IgG1, IgG2, IgG3 and IgG4 can be found in a number of sequence databases, for example, at the Uniprot database (wvww.uniprot.org) under accession numbers P01857 (IGHG1_HUMAN). P01859 (IGHG2_HUMAN), P01860 (IGHG3_HUMAN), and P01861 (IGHG1_HUMAN), respectively.

The terms “YTE” or “YTE mutant” refer to a set of mutations in an IgG1 Fc domain that results in an increase in the binding to human FcRn and improves the serum half-life of the antibody having the mutation. A YTE mutant comprises a combination of three “YTE mutations”: M252Y, S254T, and T256E, wherein the numbering is according to the EU index as in Kabat, introduced into the heavy chain of an IgG. See U.S. Pat. No. 7,658,921, which is incorporated by reference herein. The YTE mutant has been shown to increase the serum half-life of antibodies compared to wild-type versions of the same antibody. See. e.g., Dall'Acqua et al., J. Biol. Chem. 281:23514-24 (2006) and U.S. Pat. No. 7,083,784, which are hereby incorporated by reference in their entireties. A “‘Y’” mutant comprises only the M256Y mutations; similarly a “YT” mutation comprises only the M252Y and S254T; and a “YE” mutation comprises only the M252Y and T256E. It is specifically contemplated that other mutations may be present at EU positions 252 and/or 256. In certain aspects, the mutation at EU position 252 may be M252F, M252S, M252W or M252T and/or the mutation at EU position 256 may be T256S, T256R, T256Q or T256D.

The term “naturally occurring MrkA” generally refers to a state in which the MrkA protein or fragments thereof may occur. Naturally occurring MrkA means MrkA protein which is naturally produced by a cell, without prior introduction of encoding nucleic acid using recombinant technology. Thus, naturally occurring MrkA may be as produced naturally by for example K. pneumoniae and/or as isolated from different members of the Klebsiella genus.

The term “recombinant MrkA” refers to a state in which the MrkA protein or fragments thereof may occur. Recombinant MrkA means MrkA protein or fragments thereof produced by recombinant DNA, e.g., in a heterologous host. Recombinant MrkA may differ from naturally occurring MrkA by glycosylation.

Recombinant proteins expressed in prokaryotic bacterial expression systems are not glycosylated while those expressed in eukaryotic systems such as mammalian or insect cells are glycosylated. Proteins expressed in insect cells however differ in glycosylation from proteins expressed in mammalian cells.

The terms “half-life” or “in vivo half-life” as used herein refer to the biological half-life of a particular type of antibody, antigen binding protein, or polypeptide of the present disclosure in the circulation of a given animal and is represented by a time required for half the quantity administered in the animal to be cleared from the circulation and/or other tissues in the animal.

The term “subject” as used herein refers to any animal (e.g., a mammal), including, but not limited to humans, non-human primates, rodents, sheep, dogs, cats, horses, cows, bears, chickens, amphibians, reptiles, and the like, which is to be the recipient of a particular treatment. The terms “subject” and “patient” as used herein refer to any subject, particularly a mammalian subject, for whom diagnosis, prognosis, or therapy of a condition associated with a Klebsiella infection. As used herein, phrases such as “a patient having a condition associated with a Klebsiella infection” includes subjects, such as mammalian subjects, that would benefit from the administration of a therapy, imaging or other diagnostic procedure, and/or preventive treatment for that condition associated with a Klebsiella infection.

“Klebsiella” refers to a genus of gram-negative, facultatively anaerobic, rod-shaped bacteria in the Enterobacteriaceae family. Klebsiella include, for example, K. pneumoniae, K. oxyoca, K. planticola and K. granulomatis.

Members of the Klebsiella genus typically express 2 types of antigens on their cell surface: an O antigen and a K antigen. The O antigen is a lipopolysaccharide, and the K antigen is a capsular polysaccharide. The structural variability of these antigens forms the basis for their classification into Klebsiella “serotypes.” Thus, the ability of a MrkA binding protein (e.g., an antibody or an antigen binding fragment thereof) to bind to multiple serotypes refers to its ability to bind to Klebsiella with different O and/or K antigens.

The term “pharmaceutical composition” as used herein refers to a preparation which is in such form as to permit the biological activity of the active ingredient to be effective, and which contains no additional components which are unacceptably toxic to a subject to which the composition would be administered. Such composition can be sterile.

An “effective amount” of a polypeptide, e.g., an antigen binding protein (including an antibody or antigen binding fragment thereof), a MrkA polypeptide, immunogenic fragment thereof, or a polynucleotide encoding a MrkA polypeptide or an immunogenic fragment thereof, as disclosed herein is an amount sufficient to carry out a specifically stated purpose. An “effective amount” can be determined empirically and in a routine manner, in relation to the stated purpose. The term “therapeutically effective amount” as used herein refers to an amount of a polypeptide, e.g., an antigen binding protein including an antibody, or other drug effective to “treat” a disease or condition in a subject or mammal and provides some improvement or benefit to a subject having a Klebsiella-mediated disease or condition. Thus, a “therapeutically effective” amount is an amount that provides some alleviation, mitigation, and/or decrease in at least one clinical symptom of the Klebsiella-mediated disease or condition. Clinical symptoms associated with the Klebsiella-mediated disease or condition that can be treated by the methods and systems of the disclosure are well known to those skilled in the art. Further, those skilled in the art will appreciate that the therapeutic effects need not be complete or curative, as long as some benefit is provided to the subject. In some embodiments, the term “therapeutically effective” refers to an amount of a therapeutic agent that is capable of reducing MrkA activity in a patient in need thereof. The actual amount administered and rate and time-course of administration, will depend on the nature and severity of what is being treated. Prescription of treatment, e.g. decisions on dosage etc., is within the responsibility of general practitioners and other medical doctors. Appropriate doses of antibodies and antigen binding fragments thereof are well known in the art; see Ledermann J. A. et al. (1991) Int. J. Cancer 47: 659-664; Bagshawe K. D. et al. (1991) Antibody, Immunoconjugates and Radiopharmaceuticals 4: 915-922.

As used herein, a “sufficient amount” or “an amount sufficient to” achieve a particular result in a patient having a Klebsiella-mediated disease or condition refers to an amount of a therapeutic agent (e.g., an antigen binding protein including an antibody, as disclosed herein) that is effective to produce a desired effect, which is optionally a therapeutic effect (i.e., by administration of a therapeutically effective amount). In some embodiments, such particular result is a reduction in MrkA activity in a patient in need thereof.

The term “label” when used herein refers to a detectable compound or composition which is conjugated directly or indirectly to a polypeptide, e.g., an antigen binding protein including an antibody, so as to generate a “labeled” polypeptide or antibody. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” or “ameliorating” or “or ameliorate” refer to therapeutic measures that cure, slow down, lessen symptoms of, and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented. For example, the phrase “treating a patient having a Klebsiella-mediated disease or condition” refers to reducing the severity of the Klebsiella-mediated disease or condition, preferably, to an extent that the subject no longer suffers discomfort and/or altered function due to it (for example, a relative reduction in asthma exacerbations when compared to untreated patients). The phrase “preventing a Klebsiella-mediated disease or condition” refers to reducing the potential for a Klebsiella-mediated disease or condition and/or reducing the occurrence of the Klebsiella-mediated disease or condition.

An “immunologically effective amount” of a MrkA polypeptide, an immunogenic fragment thereof, or a polynucleotide encoding a MrkA polypeptide or an immunogenic fragment thereof is an amount sufficient to enhance a subject's own immune response against Klebsiella. Levels of induced immunity can be monitored, e.g., by measuring amounts of neutralizing secretory and/or serum antibodies, e.g., by complement fixation, enzyme-linked immunosorbent, serum bactericidal assay, opsonophagocytic killing assay, or biofilm formation inhibition assay.

The term “immunogenic fragment” means a fragment that generates an immune response (i.e., has immunogenic activity) when administered, alone or optionally with a suitable adjuvant, to a subject.

A “vaccine” composition according to the present invention is one comprising an immunogenically effective amount of MrkA, including immunogenically active truncates, portions, fragments and segments thereof, or a polynucleotide encoding MrkA, including immunogenically active truncates, portions, fragments and segments thereof and in any and all active combinations thereof, wherein said polypeptide, or active fragment, or fragments, or polynucleotides is/are suspended in a pharmacologically acceptable carrier, which includes all suitable diluents or excipients.

As used herein, an “immune response” refers to a response in the subject to the introduction of the MrkA polypeptide, immunogenic fragment thereof, or polynucleotide encoding MrkA polypeptide or an immunogenic fragment thereof, generally characterized by, but not limited to, production of antibodies and/or T cells. Generally, an immune response may be a cellular response such as induction or activation of CD4+ T cells or CD8+ T cells or both, specific for Klebsiella, a humoral response of increased production of anti-Klebsiella antibodies, or both cellular and humoral responses. Immune responses can also include a mucosal response, e.g., a mucosal antibody response, e.g., S-IgA production or a mucosal cell-mediated response, e.g., T-cell response.

A “protective immune response” refers to an immune response exhibited by a subject that is protective when the subject is exposed to Klebsiella. In some instances, the Klebsiella can still cause infection, but it cannot cause a serious infection. Typically, the protective immune response results in detectable levels of host engendered serum and antibodies that are capable of neutralizing Klebsiella in vitro and in vivo.

The term “adjuvant” refers to any material having the ability to (1) alter or increase the immune response to a particular antigen or (2) increase or aid an effect of a pharmacological agent. As used herein, any compound which may increase the expression, antigenicity or immunogenicity of MrkA polypeptide or immunogenic fragment thereof provided herein is a potential adjuvant.

As used herein, the term “a condition associated with a Klebsiella infection” refers to any pathology caused by (alone or in association with other mediators), exacerbated by, associated with, or prolonged by Klebsiella infection (e.g. infection with K. pneumoniae, K. oxytoca, K. planticola and/or K. granulomatis) in the subject having the disease or condition. Non-limiting examples of conditions associated with a Klebsiella infection include pneumonia, urinary tract infection, septicemia, neonatal septicemia, diarrhea, soft tissue infections, infections following an organ transplant, surgery infection, wound infection, lung infection, pyogenic liver abscesses, endophthalmitis, meningitis, necrotizing meningitis, ankylosing spondylitis and spondyloarthropathies. In some embodiments, the Klebsiella infection is a nosocomial infection. In some embodiments, the Klebsiella infection is an opportunistic infection. In some embodiments, the Klebsiella infection follows an organ transplant. In some embodiments, the subject is exposed to a Klebsiella contaminated medical device, including, e.g., a ventilator, a catheter, or an intravenous catheter.

The structure for carrying a CDR or a set of CDRs will generally be of an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to (Kabat, E. A. et al, Sequences of Proteins of Immunological Interest. 4th Edition. US Department of Health and Human Services. 1987, and updates thereof, now available on the Internet (http://immuno.bme.nwu.edu or find “Kabat” using any search engine), herein incorporated by reference. CDRs can also be carried by other scaffolds such as fibronectin or cvtochrome B.

A CDR amino acid sequence substantially as set out herein can be carried as a CDR in a human variable domain or a substantial portion thereof. The HCDR3 sequences substantially as set out herein represent embodiments of the present disclosure and each of these may be carried as a HCDR3 in a human heavy chain variable domain or a substantial portion thereof.

Variable domains employed in the disclosure can be obtained from any germ-line or rearranged human variable domain, or can be a synthetic variable domain based on consensus sequences of known human variable domains. A CDR sequence (e.g. CDR3) can be introduced into a repertoire of variable domains lacking a CDR (e.g. CDR3), using recombinant DNA technology.

For example, Marks et al. (Bio/Technology, 1992, 10:779-783; which is incorporated herein by reference) provide methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire can be combined with a CDR3 of a particular antibody. Using analogous techniques, the CDR3-derived sequences of the present disclosure can be shuffled with repertoires of VH or VL domains lacking a CDR3, and the shuffled complete VH or VL domains combined with a cognate VL or VH domain to provide antigen binding proteins. The repertoire can then be displayed in a suitable host system such as the phage display system of WO92′01047 or any of a subsequent large body of literature, including Kay, B. K., Winter, J., and McCafferty, J. (1996) Phage Display of Peptides and Proteins: A Laboratory Manual, San Diego: Academic Press, so that suitable antigen binding proteins may be selected. A repertoire can consist of from anything from 104 individual members upwards, for example from 106 to 108 or 110 members. Other suitable host systems include yeast display, bacterial display, T7 display, ribosome display and so on. For a review of ribosome display for see Lowe D and Jermutus L, 2004, Curr. Pharm, Biotech, 517-27, also WO92/01047, which are herein incorporated by reference.

Analogous shuffling or combinatorial techniques are also disclosed by Stemmer (Nature, 1994, 370:389-391, which is herein incorporated by reference), who describes the technique in relation to a β-lactamase gene but observes that the approach may be used for the generation of antibodies.

A further alternative is to generate novel VH or VL regions carrying CDR-derived sequences of the disclosure using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR. In some embodiments, one or two amino acid substitutions are made within a set of HCDRs and/or LCDRs.

Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press. Cold Spring Harbor, N.Y. (1990), all of which are herein incorporated by reference.

The skilled person will be able to use such techniques described above to provide antigen binding proteins, MrkA polypeptides, and immunogenic fragments thereof of the disclosure using routine methodology in the art.

### II. MrkA Binding Molecules

The present disclosure provides MrkA binding molecules, e.g., antibodies, antigen binding proteins, and antigen binding fragments thereof, that specifically bind MrkA, for example, Klebsiella MrkA. In some embodiments, the MrkA binding molecules, e.g., antibodies, antigen binding proteins, and antigen binding fragments thereof specifically bind to K. pneumoniae MrkA. MrkA binding molecules are referred to herein interchangeably as “MrkA binding molecules”, “MrkA binding proteins” or “MrkA binding agents”.

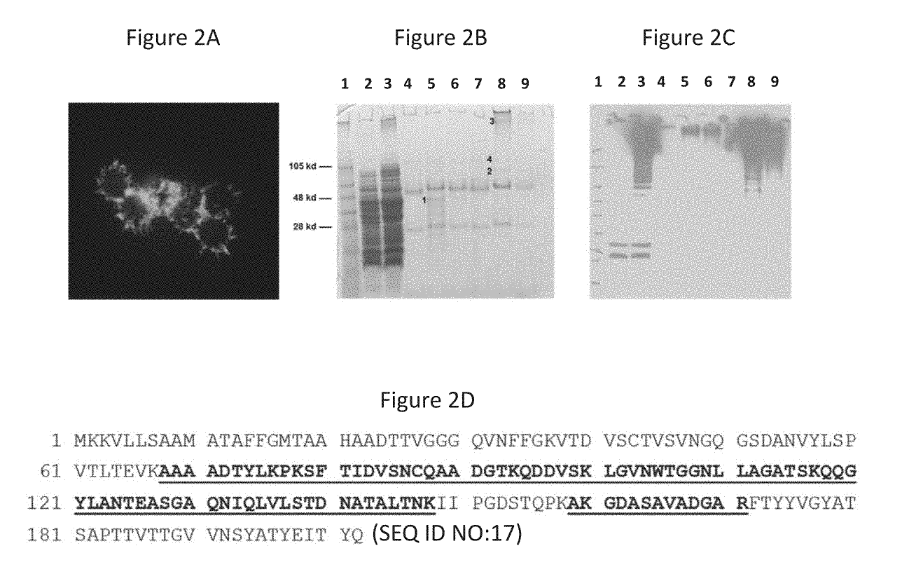

The full-length amino acid and nucleotide sequences for MrkA are known in the art (see, e.g., UniProt Acc. No. B6S767 for K. pneumoniae MrkA, or UniProt Acc. No. BOZDW4 for E. coli MrkA; both herein incorporated by reference in their entireties). As used herein, the term “K. pneumoniae MrkA” refers to the amino acid sequence shown in FIG. 2D (SEQ ID NO: 17). K. pneumoniae isolates commonly express two fimbrial adhesins, type 1 and type 3 fimbriae. The type 1 fimbriae are implicated in promoting K. pneumoniae colonization and biofilm formation, while the Type 3 fimbriae mediate biofilm formation on biotic and abiotic surfaces and are required for mature biofilm development. The various components of type 3 fimbriae are encoded by the mrkABCDF operon, which produce the major pilin subunit MrkA, chaperone MrkB, outer membrane usher MrkC, adhesin MrkD and MrkF. See Yang et al. PLoS One. 2013 Nov. 14; 8(11):e79038. Klebsiella pneumoniae type 3 fimbriae are mainly composed of MrkA pilins that assemble into a helix-like filament. The type 3 fimbriae mediate binding to target tissue using the MrkD adhesin that is associated with the fimbrial shaft comprised of the MrkA protein. See Langstraat et al., Infect Immun. 2001 September; 69(9): 5805-5812. Host cell adherence and biofilm formation of Klebsiella are mediated by such MrkA pilins. See Chan et al., Langmuir 28: 7428-7435 (2012), which is herein incorporated by reference in its entirety.

In some embodiments, the disclosure provides an isolated antigen binding protein that is an antibody or polypeptide that specifically binds to MrkA. In some embodiments, the antigen binding protein is an antigen binding fragment of an antibody that specifically binds to MrkA.

In certain embodiments, the MrkA binding molecules are antibodies or polypeptides. In some embodiments, the disclosure provides an isolated antigen binding protein thereof that is a murine, non-human, humanized, chimeric, resurfaced, or human antigen binding protein that specifically binds to MrkA. In some embodiments, the MrkA binding molecules are humanized antibodies or antigen binding fragment thereof. In some embodiments, the MrkA binding molecule is a human antibody or antigen binding fragment thereof.

The disclosure provides an isolated antigen binding protein (including, e.g., an anti-MrkA antibody or antigen binding fragment thereof) that specifically binds to MrkA, wherein said antigen binding protein (including, e.g., an anti-MrkA antibody or antigen binding fragment thereof): a) binds to at least two Klebsiella pneumoniae (K. pneumoniae) serotypes; b) induces opsonophagocytic killing (OPK) of K. pneumoniae or c) binds to at least two K. pneumoniae serotypes and induces OPK of K. pneumoniae.

In some embodiments, the disclosure provides an isolated antigen binding protein that binds to at least two K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least three K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least four K. pneumoniae serotypes selected from the group consisting of O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least five K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least six K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least seven K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least eight K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least nine K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein that binds to at least ten K. pneumoniae serotypes selected from the group consisting of: O1:K2, O1:K79, O2:K28, O2a:K28, O5:K57, O3:K58, O3:K11, O3:K25, O4:K15, O5:K61, O7:K67, and O12:K80. In some embodiments, the disclosure provides an isolated antigen binding protein (e.g., an anti-MrkA antibody or antigen binding fragment thereof) that binds to at least one, two, three, four, five, six, seven, eight, nine, or ten of the serotypes of the K. pneumoniae listed in Table 5.

In some embodiments, the disclosure provides an isolated antigen binding protein that binds to the K. pneumoniae serotypes O1:K2, O1:K79, O2:K28, O2a:K28, O5:K57, O3:K58, O3:K11, O3:K25, O4:K15, O5:K61, O7:K67, and O12:K80.

The disclosure provides an isolated antigen binding protein (including. e.g., an anti-MrkA antibody or antigen binding fragment thereof) that induces OPK of Klebsiella, including e.g., K. pneumoniae. In some embodiments, the disclosure provides an isolated antigen binding protein that induces OPK in at least one K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least two K. pneumoniae serotypes selected from the group consisting of: O1:K2, O1:K79, O2a:K28, O5:K57. O3:K58, O3:K1, O3:K25, O4:K15, O5:K61, O7:K67, and O12:K80. In some embodiments, the disclosure provides an isolated antigen binding protein that induces OPK in at least three K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least four K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least five K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least six K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least seven K. pneumoniae serotypes selected from the group consisting of: O1:K2, O1:K79, O2a:K28, O5:K57, O3:K58, O3:K1, O3:K25, O4:K15, O5:K61, O7:K67, and O12:K80. In some embodiments, the disclosure provides an isolated antigen binding protein that induces OPK in at least eight K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least nine K. pneumoniae serotypes selected from the group consisting of: O1:K2, O1:K79, O2a:K28, O5:K57, O3:K58, O3:K11, O3:K25, O4:K15, O5:K61, O7:K67, and O12:K80. In some embodiments, the disclosure provides an isolated antigen binding protein that induces OPK in at least ten K. pneumoniae serotypes selected from the group consisting of: O1:K2, O1:K79, O2a:K28, O5:K57, O3:K58, O3:K11, O3:K25, O4:K15, O5:K61, O7:K67, and O12:K80.

In some embodiments, the disclosure provides an isolated antigen binding protein (including, e.g., an anti-MrkA antibody or antigen binding fragment thereof) that induces OPK in the K. pneumoniae serotypes O1:K2, O1:K79, O2a:K28, O5:K57, O3:K58, O3:K11, O3:K25, O4:K15, O5:K61, O7:K67, and O12:K80.

In some embodiments, the disclosure provides an isolated antigen binding protein (including, e.g., an anti-MrkA antibody or antigen binding fragment thereof) that specifically binds to MrkA, wherein said antigen binding protein has at least one characteristic selected from the group consisting of: a) binds to at least two K. pneumoniae serotypes; b) induces OPK of at least one or two K. pneumoniae serotypes in vitro; c) reduces bacterial burden in a mouse Klebsiella infection model; and d) confers survival benefit in a mouse Klebsiella infection model.

In some embodiments, the disclosure provides an isolated antigen binding protein (including, e.g., an anti-MrkA antibody or antigen binding fragment thereof) that specifically binds to MrkA, wherein said antigen binding protein has at least two characteristics selected from the group consisting of: a) binds to at least two K. pneumoniae serotypes; b) induces OPK of at least one or two K. pneumoniae serotypes in vitro; c) reduces bacterial burden in a mouse Klebsiella infection model; and d) confers survival benefit in a mouse Klebsiella infection model.

In some embodiments, the disclosure provides an isolated antigen binding protein (including, e.g., an anti-MrkA antibody or antigen binding fragment thereof) that specifically binds to MrkA, wherein said antigen binding protein has at least three characteristic selected from the group consisting of: a) binds to at least two K. pneumoniae serotypes; b) induces OPK of at least one or two K. pneumoniae serotypes in vitro; c) reduces bacterial burden in a mouse Klebsiella infection model; and d) confers survival benefit in a mouse Klebsiella infection model.

In some embodiments, the disclosure provides an isolated antigen binding protein (including, e.g., an anti-MrkA antibody or antigen binding fragment thereof) that specifically binds to MrkA, wherein said antigen binding protein: a) binds to at least two K. pneumoniae serotypes; b) induces OPK of at least one or two K. pneumoniae serotypes in vitro; c) reduces bacterial burden in a mouse Klebsiella infection model; and d) confers survival benefit in a mouse Klebsiella infection model.

The MrkA-binding proteins disclosed herein include MrkA antibodies Kp3 and Kp16 and antigen-binding fragments thereof. The MrkA-binding proteins disclosed herein also include MrkA antibodies clone 1, clone 4, clone 5, and clone 6 and antigen-binding fragments thereof. The MrkA-binding proteins of the disclosure also include MrkA-binding proteins (e.g., anti-MrkA antibodies or antigen-binding fragments thereof) that specifically bind to the same MrkA epitope as Kp3 or Kp16. The MrkA-binding proteins of the disclosure also include MrkA-binding proteins (e.g., anti-MrkA antibodies or antigen-binding fragments thereof) that specifically bind to the same MrkA epitope as clone 1, clone 4, clone 5, or clone 6. In some embodiments, the disclosure provides an isolated antigen binding protein (e.g., anti-MrkA antibody or antigen-binding fragment thereof) that binds oligomeric MrkA. In some embodiments, the antigen binding protein (e.g., anti-MrkA antibody or antigen-binding fragment thereof) does not bind to monomeric MrkA. In some embodiments, the antigen binding protein (e.g., anti-MrkA antibody or antigen-binding fragment thereof) binds to monomeric MrkA (e.g., clone 1, an antibody or antigen-binding fragment thereof that contains the six CDRs or the VH and VL of clone 1, or an antibody or antigen-binding fragment thereof that binds the same epitope as or competitively inhibits binding of clone 1 to MrkA).

In some embodiments, the antigen binding protein (including e.g., an anti-MrkA antibody or antigen-binding fragment thereof) binds to an epitope within amino acids 1-40 and 171-202 of SEQ ID NO:17.

In some embodiments, the antigen binding protein (including e.g., an anti-MrkA antibody or antigen-binding fragment thereof) binds to the MrkA sequence set forth in SEQ ID NO:17, but does not bind to MrkA lacking amino acids 1-40 of SEQ ID NO:17 (i.e., SEQ ID NO:26). In some embodiments, the antigen binding protein (e.g., an anti-MrkA antibody or antigen-binding fragment thereof) binds to the MrkA sequence set forth in SEQ ID NO: 17, but does not bind to MrkA lacking amino acids 171-202 of SEQ ID NO: 17 (i.e., SEQ ID NO:27). In some embodiments, the antigen binding protein (e.g., an anti-MrkA antibody or antigen-binding fragment thereof) binds to the MrkA sequence set forth in SEQ ID NO: 17 but does not bind to MrkA lacking amino acids 1-40 and 171-202 of SEQ ID NO: 17 (i.e., SEQ ID NO:28).

In some embodiments, the antigen binding protein (e.g., an anti-MrkA antibody or antigen-binding fragment thereof) specifically binds to MrkA (SEQ ID NO: 17), but does not bind to either SEQ ID NO:26 or SEQ ID NO:27. In some embodiments, the antigen binding protein (e.g., an anti-MrkA antibody or antigen-binding fragment thereof) specifically binds to MrkA (SEQ ID NO: 17), but does not bind to any of SEQ ID NOs:26-28.

The MrkA-binding proteins (e.g. anti-MrkA antibodies or antigen binding fragments thereof) also include MrkA-binding proteins that competitively inhibit binding of Kp3 or Kp16 to MrkA. The MrkA-binding proteins (e.g. anti-MrkA antibodies or antigen binding fragments thereof) also include MrkA-binding proteins that competitively inhibit binding of clone 1, clone 4, clone 5, or clone 6 to MrkA. In some embodiments, an anti-MrkA antibody or antigen-binding fragment thereof competitively inhibits binding of Kp3 or Kp16 to MrkA in a competition ELISA assay. In some embodiments, an anti-MrkA antibody or antigen-binding fragment thereof competitively inhibits binding of clone 1, clone 4, clone 5, or clone 6 to MrkA in a competition ELISA assay. In some embodiments, an anti-MrkA antibody or antigen-binding fragment thereof competitively inhibits binding of Kp3 or Kp16 to K. pneumoniae in a competition ELISA assay. In some embodiments, an anti-MrkA antibody or antigen-binding fragment thereof competitively inhibits binding of clone 1, clone 4, clone 5, or clone 6 to K. pneumoniae in a competition ELISA assay. In some embodiments, an anti-MrkA antibody or antigen-binding fragment thereof competitively inhibits binding of Kp3 or Kp16 to K. pneumoniae strain 29011 in a competition ELISA assay. In some embodiments, an anti-MrkA antibody or antigen-binding fragment thereof competitively inhibits binding of clone 1, clone 4, clone 5, or clone 6 to K. pneumoniae strain 29011 in a competition ELISA assay. In some embodiments, an anti-MrkA antibody or antigen-binding fragment thereof competitively inhibits binding of Kp3, Kp16, clone 1, clone 4, clone 5, or clone 6 to K. pneumoniae strain 961842 in a competition ELISA assay. In some embodiments, an anti-MrkA antibody or antigen-binding fragment thereof competitively inhibits binding of Kp3, Kp16, clone 1, clone 4, clone 5, or clone 6 to K. pneumoniae strain 985048 in a competition ELISA assay.

In some embodiments, 10² fold excess of the anti-MrkA antibody or antigen-binding fragment thereof decreases binding of 1 μg Kp3 to MrkA by at least 20% in a competitive ELISA assay. In some embodiments, 10² fold excess of the anti-MrkA antibody or antigen-binding fragment thereof decreases binding of 1 μg Kp3 to MrkA by at least 25% in a competitive ELISA assay. In some embodiments, 10² fold excess of the anti-MrkA antibody or antigen-binding fragment thereof decreases binding of 1 μg Kp3 to MrkA by at least 30% in a competitive ELISA assay.

In some embodiments, 10² fold excess of the anti-MrkA antibody or antigen-binding fragment thereof decreases binding of 1 μg Kp3 to K. pneumoniae by at least 20% in a competitive ELISA assay. In some embodiments, 10² fold excess of the anti-MrkA antibody or antigen-binding fragment thereof decreases binding of 1 μg Kp3 to K. pneumoniae by at least 25% in a competitive ELISA assay. In some embodiments, 10² fold excess of the anti-MrkA antibody or antigen-binding fragment thereof decreases binding of 1 μg Kp3 to K. pneumoniae by at least 30% in a competitive ELISA assay.

In some embodiments, 10² fold excess of the anti-MrkA antibody or antigen-binding fragment thereof decreases binding of 1 μg Kp3 to K. pneumoniae strain 29011 by at least 20% in a competitive ELISA assay. In some embodiments, 10² fold excess of the anti-MrkA antibody or antigen-binding fragment thereof decreases binding of 1 μg Kp3 to K. pneumoniae strain 29011 by at least 25% in a competitive ELISA assay. In some embodiments, 10² fold excess of the anti-MrkA antibody or antigen-binding fragment thereof decreases binding of 1 μg Kp3 to K. pneumoniae strain 29011 by at least 30% in a competitive ELISA assay.

In some embodiments, the MrkA-binding proteins (including, e.g., anti-MrkA antibodies or antigen binding fragments thereof) inhibit or reduce Klebsiella biofilm formation.

In some embodiments, the MrkA-binding proteins (including, e.g., anti-MrkA antibodies or antigen binding fragments thereof) inhibit or reduce Klebsiella biofilm formation by at least 25%. In some embodiments, the MrkA-binding proteins (e.g. anti-MrkA antibodies or antigen binding fragments thereof) inhibit or reduce Klebsiella biofilm formation by at least 30%. In some embodiments, the MrkA-binding proteins (e.g. anti-MrkA antibodies or antigen binding fragments thereof) inhibit or reduce Klebsiella biofilm formation by at least 40%. In some embodiments, the MrkA-binding proteins (e.g. anti-MrkA antibodies or antigen binding fragments thereof) inhibit or reduce Klebsiella biofilm formation by at least 50%. In some embodiments, the MrkA-binding proteins (e.g. anti-MrkA antibodies or antigen binding fragments thereof) inhibit or reduce Klebsiella biofilm formation by at least 55%. In some embodiments, the MrkA-binding proteins (e.g. anti-MrkA antibodies or antigen binding fragments thereof) inhibit or reduce Klebsiella biofilm formation by at least 60%. In some embodiments, the MrkA-binding proteins (e.g. anti-MrkA antibodies or antigen binding fragments thereof) inhibit or reduce Klebsiella biofilm formation by about 25% to about 65%. In some embodiments, the MrkA-binding proteins (e.g. anti-MrkA antibodies or antigen binding fragments thereof) inhibit or reduce Klebsiella biofilm formation by about 50% to about 60%.

In some embodiments, the MrkA-binding proteins (e.g. anti-MrkA antibodies or antigen binding fragments thereof) inhibit or reduce Klebsiella biofilm formation by at least 25% at a concentration of about 3 pgml. In some embodiments, the MrkA-binding proteins (e.g. anti-MrkA antibodies or antigen binding fragments thereof) inhibit or reduce Klebsiella biofilm formation by at least 25% at a concentration of about 4 μg/ml. In some embodiments, the MrkA-binding proteins (e.g. anti-MrkA antibodies or antigen binding fragments thereof) inhibit or reduce Klebsiella biofilm formation by at least 25% at a concentration of about 5 μg/ml.

In some embodiments, the MrkA-binding proteins (e.g. anti-MrkA antibodies or antigen binding fragments thereof) inhibit or reduce Klebsiella biofilm formation by at least 50% at a concentration of about 10 μg/ml. In some embodiments, the MrkA-binding proteins (e.g. anti-MrkA antibodies or antigen binding fragments thereof) inhibit or reduce Klebsiella biofilm formation by at least 60% at a concentration of about 10 μg/ml.

In some embodiments, the MrkA-binding proteins (e.g. anti-MrkA antibodies or antigen binding fragments thereof) inhibit or reduce Klebsiella biofilm formation by about 25% to about 65% at a concentration of about 10 μg/ml. In some embodiments, the MrkA-binding proteins (e.g. anti-MrkA antibodies or antigen binding fragments thereof) inhibit or reduce Klebsiella biofilm formation by about 50% to about 60% at a concentration of about 10 μg/ml.

In some embodiments, the MrkA-binding proteins (e.g. anti-MrkA antibodies or antigen binding fragments thereof) inhibit or reduce Klebsiella cell adherence (e.g., Klebsiella epithelial cell adherence).

In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 20%. In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 30%. In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 40%. In some embodiments, the MrkA-binding proteins (e.g. anti-MrkA antibodies or antigen binding fragments thereof) inhibit or reduce Klebsiella cell adherence (e.g., Klebsiella epithelial cell adherence) by about 20% to about 50%. In some embodiments, the MrkA-binding proteins (e.g. anti-MrkA antibodies or antigen binding fragments thereof) inhibit or reduce Klebsiella cell adherence (e.g., Klebsiella epithelial cell adherence) by about 40% to about 50%.

In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 20% at a concentration of about 10 μg/ml. In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 30% at a concentration of about 10 μg/ml. In some embodiments, the MrkA-binding proteins (e.g. anti-MrkA antibodies or antigen binding fragments thereof) inhibit or reduce Klebsiella cell adherence (e.g., Klebsiella epithelial cell adherence) by at least 40% at a concentration of about 10 μg/ml. In some embodiments, the MrkA-binding proteins (e.g. anti-MrkA antibodies or antigen binding fragments thereof) inhibit or reduce Klebsiella cell adherence (e.g., Klebsiella epithelial cell adherence) by about 20% to about 50% at a concentration of about 10 μg/ml. In some embodiments, the MrkA-binding proteins (e.g. anti-MrkA antibodies or antigen binding fragments thereof) inhibit or reduce Klebsiella cell adherence (e.g., epithelial cell adherence) by about 40% to about 50% at a concentration of about 10 μg/ml.

The MrkA-binding proteins (e.g. anti-MrkA antibodies or antigen binding fragments thereof) also include MrkA-binding proteins that comprise the heavy and light chain complementarity determining region (CDR) sequences of Kp3, Kp16, clone 1, clone 4, clone 5, or clone 6. The CDR sequences of Kp3, Kp16, clone 1, clone 4, clone 5, and clone 6 are described in Tables 1 and 2 below.

TABLE 1
Variable heavy chain CDR amino acid sequences

| Antibody | VH-CDR1 | VH-CDR2 | VH-CDR3 |
|---|---|---|---|
| Kp3 | SNSNTYYWG (SEQ ID | TIHSSGRTYYNPSLKS | DLSGASLAPRRPFNYYY |
| | NO: 1) | (SEQ ID NO: 2) | YNMDV (SEQ ID NO: 3) |
| Kp16 | TYYMH (SEQ ID NO 4) | MINPSSGSTIYAQPFRG | GNYGSSFGY (SEQ ID |
| | | (SEQ ID NO: 5) | NO: 6) |
| St1_C1 | SYAVH (SEQIDNO: 29) | GINGGNGNTRISQRFQD | ADDCSGVGCHPWFDP |
| “clone 1” | | (SEQIDNO: 30) | SEQIDNO: 31) |
| St2_C4 | NANWWS (SEQIDNO: 32) | EIYHSGTTYYNPSLKS | DRDITSRGTFDV |
| “clone 4” | | (SEQIDNO: 33) | (SEQIDNO: 34) |
| St3_C5 | AYYMH (SEQIDNO: 35) | WINPSSGGTNSAQKFQG | GTIGAAGNY |
| “clone 5” | | (SEQIDNO: 36) | (SEQIDNO: 37) |
| St4_C6 | SYAVH (SEQIDNO: 38) | GVNGGNGNTRFSQKFQ | ADDCSGVGCHPWFDP |
| “clone 6” | | D (SEQIDNO: 39) | (SEQIDNO: 40) |

TABLE 2
Variable light chain CDR amino acid sequences

| Antibody | VL-CDR1 | VL-CDR2 | VL-CDR3 |
|---|---|---|---|
| Kp3 | RSSQSLVYSDGNTYLN | KVSNRDS (SEQ ID NO: 8) | MQGTHWPPIT(SEQ ID |
| | (SEQ ID NO: 7) | | NO: 9) |
| Kp16 | SGSSSNIGSNTVN(SEQ | NNNQRPS (SEQ ID | AAWDDSLNGVV (SEQ |
| | ID NO: 10) | NO: 11) | ID NO: 12) |
| St1_C1 | SGDKLGDKYVS | KDTKRPS (SEQIDNO: 42) | QAWDRSIMI |
| “clone 1” | (SEQIDNO: 41) | | (SEQIDNO: 43) |
| St2_C4 | RASEGIYHWLA | KASSLAS (SEQIDNO: 45) | QQYSNYPLT |
| “clone 4” | (SEQIDNO: 44) | | (SEQIDNO: 46) |
| St3_C5 | SGSRPNIGGNTVN | SNSQRPS (SEQIDNO: 48) | AAWDDSLTGPV |
| “clone 5” | (SEQIDNO: 47) | | (SEQIDNO: 49) |
| St4_C6 | SGDKLGDKYTS | QDTKRPS (SEQIDNO: 51) | QAWDSDSGTAT |
| “clone 6” | (SEQIDNO: 50) | | (SEQIDNO: 52) |

Antigen binding proteins (including anti-MrkA antibodies or antigen binding fragments thereof) described herein can comprise one of the individual variable light chains or variable heavy chains described herein. Antigen binding proteins (including anti-MrkA antibodies or antigen binding fragments thereof) described herein can also comprise both a variable light chain and a variable heavy chain. The variable light chain and variable heavy chain sequences of anti-MrkA Kp3, Kp16, clone 1, clone 4, clone 5, and clone 6 antibodies are provided in Tables 3 and 4 below.

TABLE 3
Variable heavy chain amino acid sequences

| Antibody | VH Amino Acid Sequence (SEQ ID NO) |
|---|---|
| Kp3 | QVQLQESGPGLVKPSETLSLTCTVSGGSMNSNSNTYYWGWIRQPPGKGLEWIGTIH |
| | SSGRTYYNPSLKSRVTISVDMSKNQFSLNLTSATAADTAVYYCARDLSGASLAPRR |
| | PFNYYYYNMDVWGRGTLVTVSS (SEQ ID NO: 13) |
| Kpl6 | QVQLQQSGAEVKKPGASVKVSCKASGYALTTYYMHWVRQAPGQGLQWMGMIN |
| | PSSGSTIYAQPERGRVTLTRDTSSGTVFMDLSSLTSEDTAIYYCARGNYGSSFGYW |
| | GKGTMVTVSS (SEQ ID NO: 14) |
| St1_C1 | QVQLVQSGAEVRKPGASVTVFCRTSGYIFTSYAVHWVRQAPGQGLEWMGGINGG |
| “clone 1” | NGNTRISQRFQDRLMITRDRSANTASMELRSLTSEDTAIYYCARADDCSGVGCHP |
| | WFDPWGRGTLVTVSS (SEQIDNO: 53) |
| St2_C4 | QLQLQESGPGLVKPSGTLSLTCAVSGDSIDNANWWSWVRQTPGKGLEWIGEIYHS |
| “clone 4” | GTTYYNPSLKSRVTISIDNSKNQFSLALTSVTAADTAVYYCARDRDITSRGTFDVW |
| | GRGTMVTVSS (SEQIDNO: 54) |
| St3_C5 | QVQLVQSGAEVKKPGASLKVSCKASGYTFTAYYMHWVRQAPGHGLEWMGWINP |
| “clone 5” | SSGGTNSAQKFQGRVTMTRDTSINTAYMELSRLTSDDTAVYYCARGTIGAAGNY |
| | WGQGTLVTVSS (SEQIDNO: 55) |
| St4_C6 | QVQLVQSGAEVRKPGASVTLSCRTSGYTFTSYAVHWVRQAPGQGLEWMGGVNG |
| “clone 6” | GNGNTRFSQKFQDRLMIVRDRSANTASMELRSLTSEDTAVYYCARADDCSGVGC |
| | HPWFDPWGQGTLVTVSS (SEQIDNO: 56) |

TABLE 4
Variable light chain amino acid sequences

| Antibody | VL Amino Acid Sequence (SEQ ID NO) |
|---|---|
| Kp3 | DVVMTQSPLSLPVTLGQPASISCRSSQSLVYSDGNTYLNWFQQRPGQSPRRLIYKV |
| | SNRDSGVPDRFSGSGSGTDFTLKISRVEAEDVGVYYCMQGTHWPPITFGQGTRLEI |
| | K (SEQ ID NO: 15) |
| Kp16 | SYVLTQPPSASGTPCQRVTISCSGSSSNIGSNTVNWYQQLPGTAPKLLIYNNNQRPS |
| | GVPDRFSGSKSGTSASLAISGLQSEDEADYYCAAWDDSLNGVVFGGGTKVTVL |
| | (SEQ ID NO: 16) |
| St1_C1 | QSVLTQPPSVSVSPGHTASITCSGDKLGDKYVSWYQQKSGQSPVLVMYKDTKRPS |
| “clone 1” | GIPERFSGSNSGNTATLAISGTQAVDEADYFCQAWDRSIMIFGGGTKVTVL (SEQ |
| | ID NO: 57) |
| St2_C4 | DIQMTQSPSTLSASIGDRVTITCRASEGIYHWLAWYQQKPGKAPKLLIYKASSLASG |
| “clone 4” | APSRFSGSGSGTDFTLTISSLQPDDFATYYCQQYSNYPLTGGGTKLEIK |
| | (SEQIDNO: 58) |
| St3_C5 | QSVLTQPPSASGTPGQRVTISCSGSRPNIGGNTVNWYQQLPGAAPKLLIYSNSQRPS |
| “clone 5” | GVPDRFSGSKYGTSASLAISGLQSDDEADYYCAAWDDSLTGPVFGGGTKLTIL |
| | (SEQIDNO: 59) |
| St4_C6 | SVILTQPPSVSVSPGQTANITCSGDKLGDKYTSWYLQKPGQSPVLLIFQDTKRPSDIP |
| “clone 6” | ERFSGSNSGNTATLTISGTQAVDEADYYCQAWDSDSGTATFGGGTKLTVL |
| | (SEQIDNO: 60) |

In some embodiments, the disclosure provides an isolated antigen binding protein (including anti-MrkA antibodies or antigen binding fragments thereof) that specifically binds to MrkA, wherein said antigen binding protein comprises a heavy chain variable region (VH) at least 95, 96, 97, 98, or 99% identical to SEQ ID NOs: 13-14 or 53-56 and a light chain variable region (VL) at least 95, 96, 97, 98, or 99% identical to SEQ ID NOs: 15-16 or 57-60. In some embodiments, the isolated antigen binding protein that specifically binds to MrkA comprises a heavy chain variable region comprising the sequences of SEQ ID NOs: 13-14 or 53-56 and a light chain variable region comprising the sequences of SEQ ID NOs:15-16 or 57-60. In some embodiments, the polypeptide having a certain percentage of sequence identity to SEQ ID NOs:13-16 or 53-60 differs from SEQ ID NOs:13-16 or 53-60 by conservative amino acid substitutions only.

In some embodiments, the disclosure provides an isolated antigen binding protein (including anti-MrkA antibodies or antigen binding fragments thereof) that specifically binds to MrkA, wherein said antigen binding protein comprises a VH at least 95% identical to SEQ ID NO:13 and a VL at least 95% identical to SEQ ID NO:15, a VH at least 95% identical to SEQ ID NO:14 and a VL at least 95% identical to SEQ ID NO:16, a VH at least 95% identical to SEQ ID NO:53 and a VL at least 95% identical to SEQ ID NO:57, a VH at least 95% identical to SEQ ID NO:54 and a VL at least 95% identical to SEQ ID NO:58, a VH at least 95% identical to SEQ ID NO:55 and a VL at least 95% identical to SEQ ID NO:59, or a VH at least 95% identical to SEQ ID NO:56 and a VL at least 95% identical to SEQ ID NO:60, wherein the antigen binding protein binds to at least two K. pneumoniae serotypes.

In some embodiments, the disclosure provides an isolated antigen binding protein (including anti-MrkA antibodies or antigen binding fragments thereof) that specifically binds to MrkA, wherein said antigen binding protein comprises a VH at least 95% identical to SEQ ID NO: 13 and a VL at least 95% identical to SEQ ID NO: 15, a VH at least 95% identical to SEQ ID NO: 14 and a VL at least 95% identical to SEQ ID NO: 16, a VH at least 95% identical to SEQ ID NO:53 and a VL at least 95% identical to SEQ ID NO:57, a VH at least 95% identical to SEQ ID NO:54 and a VL at least 95% identical to SEQ ID NO:58, a VH at least 95% identical to SEQ ID NO:55 and a VL at least 95% identical to SEQ ID NO:59, or a VH at least 95% identical to SEQ ID NO:56 and a VL at least 95% identical to SEQ ID NO:60, wherein the antigen binding protein induces OPK of at least two K. pneumoniae serotypes in vitro.

In some embodiments, the disclosure provides an isolated antigen binding protein (including anti-MrkA antibodies or antigen binding fragments thereof) that specifically binds to MrkA, wherein said antigen binding protein comprises a VH at least 95% identical to SEQ ID NO:13 and a VL at least 95% identical to SEQ ID NO:15, a VH at least 95% identical to SEQ ID NO: 14 and a VL at least 95% identical to SEQ ID NO: 16, a VH at least 95% identical to SEQ ID NO:53 and a VL at least 95% identical to SEQ ID NO:57, a VH at least 95% identical to SEQ ID NO:54 and a VL at least 95% identical to SEQ ID NO:58, a VH at least 95% identical to SEQ ID NO:55 and a VL at least 95% identical to SEQ ID NO:59, or a VH at least 95% identical to SEQ ID NO:56 and a VL at least 95% identical to SEQ ID NO:60, wherein the antigen binding protein reduces bacterial burden in a subject.

In some embodiments, the disclosure provides an isolated antigen binding protein (including anti-MrkA antibodies or antigen binding fragments thereof) that specifically binds to MrkA, wherein said antigen binding protein comprises a VH at least 95% identical to SEQ ID NO: 13 and a VL at least 95% identical to SEQ ID NO: 15, a VH at least 95% identical to SEQ ID NO: 14 and a VL at least 95% identical to SEQ ID NO: 16, a VH at least 95% identical to SEQ ID NO:53 and a VL at least 95% identical to SEQ ID NO:57, a VH at least 95% identical to SEQ ID NO:54 and a VL at least 95% identical to SEQ ID NO:58, a VH at least 95% identical to SEQ ID NO:55 and a VL at least 95% identical to SEQ ID NO:59, or a VH at least 95% identical to SEQ ID NO:56 and a VL at least 95% identical to SEQ ID NO:60, wherein the antigen binding protein confers survival benefit in a subject.

In some embodiments, the disclosure provides an isolated antigen binding protein (including anti-MrkA antibodies or antigen binding fragments thereof) that specifically binds to MrkA, wherein said antigen binding protein comprises a VH at least 96% identical to SEQ ID NO:13 and a VL at least 96% identical to SEQ ID NO:15, a VH at least 96% identical to SEQ ID NO:14 and a VL at least 96% identical to SEQ ID NO:16, a VH at least 96% identical to SEQ ID NO:53 and a VL at least 96% identical to SEQ ID NO:57, a VH at least 96% identical to SEQ ID NO:54 and a VL at least 96% identical to SEQ ID NO:58, a VH at least 96% identical to SEQ ID NO:55 and a VL at least 96% identical to SEQ ID NO:59, or a VH at least 96% identical to SEQ ID NO:56 and a VL at least 96% identical to SEQ ID NO:60, wherein the antigen binding protein binds to at least two K. pneumoniae serotypes.

In some embodiments, the disclosure provides an isolated antigen binding protein (including anti-MrkA antibodies or antigen binding fragments thereof) that specifically binds to MrkA, wherein said antigen binding protein comprises a VH at least 96% identical to SEQ ID NO: 13 and a VL at least 96% identical to SEQ ID NO: 15, a VH at least 96% identical to SEQ ID NO: 14 and a VL at least 96% identical to SEQ ID NO: 16, a VH at least 96% identical to SEQ ID NO:53 and a VL at least 96% identical to SEQ ID NO:57, a VH at least 96% identical to SEQ ID NO:54 and a VL at least 96% identical to SEQ ID NO:58, a VH at least 96% identical to SEQ ID NO:55 and a VL at least 96% identical to SEQ ID NO:59, or a VH at least 96% identical to SEQ ID NO:56 and a VL at least 96% identical to SEQ ID NO:60, wherein the antigen binding protein induces OPK of at least two K. pneumoniae serotypes in vitro.

In some embodiments, the disclosure provides an isolated antigen binding protein (including anti-MrkA antibodies or antigen binding fragments thereof) that specifically binds to MrkA, wherein said antigen binding protein comprises a VH at least 96% identical to SEQ ID NO: 13 and a VL at least 96% identical to SEQ ID NO: 15, a VH at least 96% identical to SEQ ID NO: 14 and a VL at least 96% identical to SEQ ID NO: 16, a VH at least 96% identical to SEQ ID NO:53 and a VL at least 96% identical to SEQ ID NO:57, a VH at least 96% identical to SEQ ID NO:54 and a VL at least 96% identical to SEQ ID NO:58, a VH at least 96% identical to SEQ ID NO:55 and a VL at least 96% identical to SEQ ID NO:59, or a VH at least 96% identical to SEQ ID NO:56 and a VL at least 96% identical to SEQ ID NO:60, wherein the antigen binding protein reduces bacterial burden in a subject.

In some embodiments, the disclosure provides an isolated antigen binding protein (including anti-MrkA antibodies or antigen binding fragments thereof) that specifically binds to MrkA, wherein said antigen binding protein comprises a VH at least 96% identical to SEQ ID NO: 13 and a VL at least 96% identical to SEQ ID NO: 15, a VH at least 96% identical to SEQ ID NO: 14 and a VL at least 96% identical to SEQ ID NO: 16, a VH at least 96% identical to SEQ ID NO:53 and a VL at least 96% identical to SEQ ID NO:57, a VH at least 96% identical to SEQ ID NO:54 and a VL at least 96% identical to SEQ ID NO:58, a VH at least 96% identical to SEQ ID NO:55 and a VL at least 96% identical to SEQ ID NO:59, or a VH at least 96% identical to SEQ ID NO:56 and a VL at least 96% identical to SEQ ID NO:60, wherein the antigen binding protein confers survival benefit in a subject.

In some embodiments, the disclosure provides an isolated antigen binding protein (including anti-MrkA antibodies or antigen binding fragments thereof) that specifically binds to MrkA, wherein said antigen binding protein comprises a VH at least 97% identical to SEQ ID NO: 13 and a VL at least 97% identical to SEQ ID NO: 15, a VH at least 97% identical to SEQ ID NO: 14 and a VL at least 97% identical to SEQ ID NO: 16, a VH at least 97% identical to SEQ ID NO:53 and a VL at least 97% identical to SEQ ID NO:57, a VH at least 97% identical to SEQ ID NO:54 and a VL at least 97% identical to SEQ ID NO:58, a VH at least 97% identical to SEQ ID NO:55 and a VL at least 97% identical to SEQ ID NO:59, or a VH at least 97% identical to SEQ ID NO:56 and a VL at least 97% identical to SEQ ID NO:60, wherein the antigen binding protein binds to at least two K. pneumoniae serotypes.

In some embodiments, the disclosure provides an isolated antigen binding protein (including anti-MrkA antibodies or antigen binding fragments thereof) that specifically binds to MrkA, wherein said antigen binding protein comprises a VH at least 97% identical to SEQ ID NO:13 and a VL at least 97% identical to SEQ ID NO:15, a VH at least 97% identical to SEQ ID NO: 14 and a VL at least 97% identical to SEQ ID NO: 16, a VH at least 97% identical to SEQ ID NO:53 and a VL at least 97% identical to SEQ ID NO:57, a VH at least 97% identical to SEQ ID NO:54 and a VL at least 97% identical to SEQ ID NO:58, a VH at least 97% identical to SEQ ID NO:55 and a VL at least 97% identical to SEQ ID NO:59, or a VH at least 97% identical to SEQ ID NO:56 and a VL at least 97% identical to SEQ ID NO:60, wherein the antigen binding protein induces OPK of at least two K. pneumoniae serotypes in vitro.

In some embodiments, the disclosure provides an isolated antigen binding protein (including anti-MrkA antibodies or antigen binding fragments thereof) that specifically binds to MrkA, wherein said antigen binding protein comprises a VH at least 97% identical to SEQ ID NO: 13 and a VL at least 97% identical to SEQ ID NO: 15, a VH at least 97% identical to SEQ ID NO: 14 and a VL at least 97% identical to SEQ ID NO: 16, a VH at least 97% identical to SEQ ID NO:53 and a VL at least 97% identical to SEQ ID NO:57, a VH at least 97% identical to SEQ ID NO:54 and a VL at least 97% identical to SEQ ID NO:58, a VH at least 97%0 identical to SEQ ID NO:55 and a VL at least 97% identical to SEQ ID NO:59, or a VH at least 97% identical to SEQ ID NO:56 and a VL at least 97% identical to SEQ ID NO:60, wherein the antigen binding protein reduces bacterial burden in a subject.

In some embodiments, the disclosure provides an isolated antigen binding protein (including anti-MrkA antibodies or antigen binding fragments thereof) that specifically binds to MrkA, wherein said antigen binding protein comprises a VH at least 97% identical to SEQ ID NO: 13 and a VL at least 97% identical to SEQ ID NO: 15, a VH at least 97% identical to SEQ ID NO: 14 and a VL at least 97% identical to SEQ ID NO: 16, a VH at least 97% identical to SEQ ID NO:53 and a VL at least 97% identical to SEQ ID NO:57, a VH at least 97% identical to SEQ ID NO:54 and a VL at least 97% identical to SEQ ID NO:58, a VH at least 97% identical to SEQ ID NO:55 and a VL at least 97% identical to SEQ ID NO:59, or a VH at least 97% identical to SEQ ID NO:56 and a VL at least 97% identical to SEQ ID NO:60, wherein the antigen binding protein confers survival benefit in a subject.

In some embodiments, the disclosure provides an isolated antigen binding protein (including anti-MrkA antibodies or antigen binding fragments thereof) that specifically binds to MrkA, wherein said antigen binding protein comprises a VH at least 98% identical to SEQ ID NO:13 and a VL at least 98% identical to SEQ ID NO:15, a VH at least 98% identical to SEQ ID NO:14 and a VL at least 98% identical to SEQ ID NO:16, a VH at least 98% identical to SEQ ID NO:53 and a VL at least 98% identical to SEQ ID NO:57, a VH at least 98% identical to SEQ ID NO:54 and a VL at least 98% identical to SEQ ID NO:58, a VH at least 98% identical to SEQ ID NO:55 and a VL at least 98% identical to SEQ ID NO:59, or a VH at least 98% identical to SEQ ID NO:56 and a VL at least 98% identical to SEQ ID NO:60, wherein the antigen binding protein binds to at least two K. pneumoniae serotypes.

In some embodiments, the disclosure provides an isolated antigen binding protein (including anti-MrkA antibodies or antigen binding fragments thereof) that specifically binds to MrkA, wherein said antigen binding protein comprises a VH at least 98% identical to SEQ ID NO: 13 and a VL at least 98% identical to SEQ ID NO: 15, a VH at least 98% identical to SEQ ID NO: 14 and a VL at least 98% identical to SEQ ID NO: 16, a VH at least 98% identical to SEQ ID NO:53 and a VL at least 98% identical to SEQ ID NO:57, a VH at least 98% identical to SEQ ID NO:54 and a VL at least 98% identical to SEQ ID NO:58, a VH at least 98% identical to SEQ ID NO:55 and a VL at least 98% identical to SEQ ID NO:59, or a VH at least 98% identical to SEQ ID NO:56 and a VL at least 98% identical to SEQ ID NO:60, wherein the antigen binding protein induces OPK of at least two K. pneumoniae serotypes in vitro.

In some embodiments, the disclosure provides an isolated antigen binding protein (including anti-MrkA antibodies or antigen binding fragments thereof) that specifically binds to MrkA, wherein said antigen binding protein comprises a VH at least 98% identical to SEQ ID NO: 13 and a VL at least 98% identical to SEQ ID NO: 15, a VH at least 98% identical to SEQ ID NO: 14 and a VL at least 98% identical to SEQ ID NO: 16, a VH at least 98% identical to SEQ ID NO:53 and a VL at least 98% identical to SEQ ID NO:57, a VH at least 98% identical to SEQ ID NO:54 and a VL at least 98% identical to SEQ ID NO:58, a VH at least 98% identical to SEQ ID NO:55 and a VL at least 98% identical to SEQ ID NO:59, or a VH at least 98% identical to SEQ ID NO:56 and a VL at least 98% identical to SEQ ID NO:60, wherein the antigen binding protein reduces bacterial burden in a subject.

In some embodiments, the disclosure provides an isolated antigen binding protein (including anti-MrkA antibodies or antigen binding fragments thereof) that specifically binds to MrkA, wherein said antigen binding protein comprises a VH at least 98% identical to SEQ ID NO:13 and a VL at least 98% identical to SEQ ID NO:15, a VH at least 98% identical to SEQ ID NO:14 and a VL at least 98% identical to SEQ ID NO:16, a VH at least 98% identical to SEQ ID NO:53 and a VL at least 98% identical to SEQ ID NO:57, a VH at least 98% identical to SEQ ID NO:54 and a VL at least 98% identical to SEQ ID NO:58, a VH at least 98% identical to SEQ ID NO:55 and a VL at least 98% identical to SEQ ID NO:59, or a VH at least 98% identical to SEQ ID NO:56 and a VL at least 98% identical to SEQ ID NO:60, wherein the antigen binding protein confers survival benefit in a subject.

In some embodiments, the disclosure provides an isolated antigen binding protein (including anti-MrkA antibodies or antigen binding fragments thereof) that specifically binds to MrkA, wherein said antigen binding protein comprises a VH at least 99% identical to SEQ ID NO: 13 and a VL at least 99% identical to SEQ ID NO: 15, a VH at least 99% identical to SEQ ID NO: 14 and a VL at least 99%⁰ identical to SEQ ID NO: 16, a VH at least 99% identical to SEQ ID NO:53 and a VL at least 99% identical to SEQ ID NO:57, a VH at least 99% identical to SEQ ID NO:54 and a VL at least 99% identical to SEQ ID NO:58, a VH at least 99% identical to SEQ ID NO:55 and a VL at least 99% identical to SEQ ID NO:59, or a VH at least 99% identical to SEQ ID NO:56 and a VL at least 99% identical to SEQ ID NO:60, wherein the antigen binding protein binds to at least two K. pneumoniae serotypes.

In some embodiments, the disclosure provides an isolated antigen binding protein (including anti-MrkA antibodies or antigen binding fragments thereof) that specifically binds to MrkA, wherein said antigen binding protein comprises a VH at least 99% identical to SEQ ID NO: 13 and a VL at least 99% identical to SEQ ID NO: 15, a VH at least 99% identical to SEQ ID NO: 14 and a VL at least 99% identical to SEQ ID NO: 16, a VH at least 990/identical to SEQ ID NO:53 and a VL at least 99% identical to SEQ ID NO:57, a VH at least 99% identical to SEQ ID NO:54 and a VL at least 99% identical to SEQ ID NO:58, a VH at least 99% identical to SEQ ID NO:55 and a VL at least 99% identical to SEQ ID NO:59, or a VH at least 99% identical to SEQ ID NO:56 and a VL at least 99% identical to SEQ ID NO:60, wherein the antigen binding protein induces OPK of at least two K. pneumoniae serotypes in vitro.

In some embodiments, the disclosure provides an isolated antigen binding protein (including anti-MrkA antibodies or antigen binding fragments thereof) that specifically binds to MrkA, wherein said antigen binding protein comprises a VH at least 99% identical to SEQ ID NO: 13 and a VL at least 99% identical to SEQ ID NO: 15, a VH at least 99% identical to SEQ ID NO: 14 and a VL at least 99% identical to SEQ ID NO: 16, a VH at least 99% identical to SEQ ID NO:53 and a VL at least 99% identical to SEQ ID NO:57, a VH at least 99% identical to SEQ ID NO:54 and a VL at least 99% identical to SEQ ID NO:58, a VH at least 99% identical to SEQ ID NO:55 and a VL at least 99% identical to SEQ ID NO:59, or a VH at least 99% identical to SEQ ID NO:56 and a VL at least 99% identical to SEQ ID NO:60, wherein the antigen binding protein reduces bacterial burden in a subject.

In some embodiments, the disclosure provides an isolated antigen binding protein (including anti-MrkA antibodies or antigen binding fragments thereof) that specifically binds to MrkA, wherein said antigen binding protein comprises a VH at least 99% identical to SEQ ID NO: 13 and a VL at least 99° % identical to SEQ ID NO: 15, a VH at least 99% identical to SEQ ID NO: 14 and a VL at least 99% identical to SEQ ID NO: 16, a VH at least 99% identical to SEQ ID NO:53 and a VL at least 99% identical to SEQ ID NO:57, a VH at least 99% identical to SEQ ID NO:54 and a VL at least 99% identical to SEQ ID NO:58, a VH at least 99% identical to SEQ ID NO:55 and a VL at least 99% identical to SEQ ID NO:59, or a VH at least 99% identical to SEQ ID NO:56 and a VL at least 99% identical to SEQ ID NO:60, wherein the antigen binding protein confers survival benefit in a subject.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo in an animal. The monoclonal antibodies can then be purified from the culture medium or ascites fluid.

Alternatively monoclonal antibodies can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. ofa monoclonal antibody.

In some embodiments, the monoclonal antibody against the MrkA is a humanized antibody. In certain embodiments, such antibodies are used therapeutically to reduce antigenicity and HAMA (human anti-mouse antibody) responses when administered to a human subject. Humanized antibodies can be produced using various techniques known in the art. In certain alternative embodiments, the antibody to MrkA is a human antibody.

Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373). Also, the human antibody can be selected from a phage library, where that phage library expresses human antibodies, as described, for example, in Vaughan et al., 1996, Nat. Biotech., 14:309-314, Sheets et al., 1998, Proc. Nat'l. Acad. Sci., 95:6157-6162, Hoogenboom and Winter, 1991, J. Mol. Biol., 227:381, and Marks et al., 1991, J. Mol. Biol., 222:581). Techniques for the generation and use of antibody phage libraries are also described in U.S. Pat. Nos. 5,969,108, 6,172,197, 5,885,793, 6,521,404; 6,544.731; 6,555,313; 6,582,915; 6,593,081; 6,300,064; 6,653,068; 6,706,484; and 7,264.963; and Rothe et al., 2007. J. Mol. Bio., doi: 10.1016/j.jmb.2007.12.018 (each of which is incorporated by reference in its entirety). Affinity maturation strategies and chain shuffling strategies (Marks et al., 1992, BioFfechnology 10:779-783, incorporated by reference in its entirety) are known in the art and can be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625.126; 5,633,425; and 5,661,016.

According to the present disclosure, techniques can be adapted for the production of single-chain antibodies specific to MrkA (see U.S. Pat. No. 4,946,778). In addition, methods can be adapted for the construction of Fab expression libraries (Huse, et al., Science 246:1275-1281 (1989)) to allow rapid and effective identification of monoclonal Fab fragments with the desired specificity for MrkA, or fragments thereof. Antibody fragments can be produced by techniques in the art including, but not limited to: (a) a F(ab′)2 fragment produced by pepsin digestion of an antibody molecule, (b) a Fab fragment generated by reducing the disulfide bridges of an F(ab′)2 fragment, (c) a Fab fragment generated by the treatment of the antibody molecule with papain and a reducing agent, and (d) Fv fragments.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antigen binding proteins of the present disclosure can further comprise antibody constant regions or parts thereof. For example, a VL domain can be attached at its C-terminal end to antibody light chain constant domains including human CK or Cy chains. Similarly, an antigen binding protein based on a VH domain can be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. For example, the immunoglobulin heavy chain can be derived from the antibody isotype sub-class. IgG1. Any synthetic or other constant region variant that has these properties and stabilizes variable regions is also contemplated for use in embodiments of the present disclosure. The antibody constant region can be an Fc region with a YTE mutation, such that the Fc region comprises the following amino acid substitutions: M252Y/S254T/T256E. This residue numbering is based on Kabat numbering. The YTE mutation in the Fc region increases serum persistence of the antigen-binding protein (see Dall'Acqua, W. F. et al. (2006) The Journal of Biological Chemistry, 281, 23514-23524).

In some embodiments herein, the antigen binding protein, e.g., antibody or antigen-binding fragment thereof is modified to improve effector function, e.g., so as to enhance antigen-dependent cell-mediated cytotoxicity (ADCC) and/or complement dependent cytotoxicity (CDC). This can be achieved by making one or more amino acid substitutions or by introducing cysteine in the Fc region. Variants of the Fc region (e.g., amino acid substitutions and/or additions and/or deletions) that can enhance or diminish effector function of an antibody and/or alter the pharmacokinetic properties (e.g., half-life) of the antibody are disclosed, for example in U.S. Pat. No. 6,737,056B1, U.S. Patent Application Publication No. 2004/0132101A1, U.S. Pat. No. 6,194,551, and U.S. Pat. Nos. 5,624,821 and 5,648,260. One particular set of substitutions, the triple mutation L234F/L235E/P331S (“TM”) causes a profound decrease in the binding activity of human IgG1 molecules to human Clq, CD64, CD32A and CD16. See, e.g., Oganesyan et al., Acta Crystallogr D Biol Crystallogr. 64:700-704 (2008). In other cases it can be that constant region modifications increase serum half-life. The serum half-life of proteins comprising Fc regions can be increased by increasing the binding affinity of the Fc region for FcRn.

When the antigen-binding protein is an antibody or an antigen-binding fragment thereof, it can further comprise a heavy chain immunoglobulin constant domain selected from the group consisting of: (a) an IgA constant domain; (b) an IgD constant domain; (c) an IgE constant domain; (d) an IgG1 constant domain; (e) an IgG2 constant domain; (f) an IgG3 constant domain; (g) an IgG4 constant domain; and (h) an IgM constant domain. In some embodiments, the antigen-binging protein is an antibody or an antigen-binding fragment thereof that comprises an IgG1 heavy chain immunoglobulin constant domain. In some embodiments, the antigen-binding protein is an antibody or an antigen-binding fragment thereof that comprises an IgG1/IgG3 chimeric heavy chain immunoglobulin constant domain.

The antigen-binding protein of the disclosure can further comprise a light chain immunoglobulin constant domain selected from the group consisting of: (a) an Ig kappa constant domain; and (b) an Ig lambda constant domain.

The antigen-binding protein of the disclosure can further comprise a human IgG1 constant domain and a human lambda constant domain.

The antigen-binding protein of the disclosure can comprise an IgG Fc domain containing a mutation at positions 252, 254 and 256, wherein the position numbering is according to the EU index as in Kabat. For example, the IgG1 Fc domain can contain a mutation of M252Y. S254T, and T256E, wherein the position numbering is according to the EU index as in Kabat.

The present disclosure also relates to an isolated VH domain of the antigen-binding protein of the disclosure and/or an isolated VL domain of the antigen-binding protein of the disclosure.

Antigen-binding proteins (including anti-MrkA antibodies or antigen binding fragments thereof) of the disclosure can be labeled with a detectable or functional label. Detectable labels include radiolabels such as 1311 or 99Tc, which may be attached to antibodies of the present disclosure using conventional chemistry known in the art of antibody imaging. Labels also include enzyme labels such as horseradish peroxidase. Labels further include chemical moieties such as biotin which may be detected via binding to a specific cognate detectable moiety, e.g., labeled avidin. Non-limiting examples of other detectable or functional labels which may be attached to the antigen-binding proteins (including antibodies or antigen binding fragments thereof) of the disclosure include: isotopic labels, magnetic labels, redox active moieties, optical dyes, biotinylated groups, fluorescent moieties such as biotin signaling peptides, Green Fluorescent Proteins (GFPs), blue fluorescent proteins (BFPs), cyan fluorescent proteins (CFPs), and yellow fluorescent proteins (YFPs), and polypeptide epitopes recognized by a secondary reporter such as histidine peptide (his), hemagglutinin (HA), gold binding peptide, Flag; a radioisotope, radionuclide, a toxin, a therapeutic and a chemotherapeutic agent.

### III. Pharmaceutical Compositions and Vaccines

The disclosure also provides a pharmaceutical composition comprising one or more of the MrkA-binding agents (including, e.g., anti-MrkA antibodies or antigen binding fragments) described herein, a MrkA polypeptide, an immunogenic fragment thereof, or a polynucleotide encoding a MrkA polypeptide or an immunogenic fragment thereof. In certain embodiments, the pharmaceutical compositions further comprise a pharmaceutically acceptable vehicle or pharmaceutically acceptable excipient. In certain embodiments, these pharmaceutical compositions find use in treating, preventing or ameliorating a condition associated with a Klebsiella infection in human patients. In certain embodiments, these pharmaceutical compositions find use in inhibiting growth of Klebsiella.

In certain embodiments, formulations are prepared for storage and use by combining an antibody or anti-MrkA binding agent, a MrkA polypeptide, an immunogenic fragment thereof, or a polynucleotide encoding a MrkA polypeptide or an immunogenic fragment thereof described herein with a pharmaceutically acceptable vehicle (e.g., carrier, excipient) (see, e.g., Remington, The Science and Practice of Pharmacy 20th Edition Mack Publishing, 2000, herein incorporated by reference). In some embodiments, the formulation comprises a preservative.

The pharmaceutical compositions of the present disclosure can be administered in any number of ways for either local or systemic treatment.

In some embodiments, a pharmaceutical composition comprising one or more of the MrkA-binding agents (including, e.g., anti-MrkA antibodies or antigen binding fragments), MrkA polypeptides, immunogenic fragments thereof, or polynucleotides encoding MrkA polypeptides or immunogenic fragments thereof described herein is used for treating pneumonia, urinary tract infection, septicemia, neonatal septicemia, diarrhea, soft tissue infection, infection following an organ transplant, surgery infection, wound infection, lung infection, pyogenic liver abscesses (PLA), endophthalmitis, meningitis, necrotizing meningitis, ankylosing spondvlitis, or spondyloarthropathies. In some embodiments, a pharmaceutical composition comprising one or more of the MrkA-binding agents (including, e.g., anti-MrkA antibodies or antigen binding fragments), MrkA polypeptides, immunogenic fragments thereof, or polynucleotides encoding MrkA polypeptides or immunogenic fragments thereof described herein is useful in nosocomial infections, opportunistic infections, infections following organ transplants, and other conditions associated with a Klebsiella infection (e.g. infection with K. pneumoniae, K. oxytoca, K. planticola, and/or K. granulomatis). In some embodiments, a pharmaceutical composition comprising one or more of the MrkA-binding agents (including, e.g., anti-MrkA antibodies or antigen binding fragments), MrkA polypeptides, immunogenic fragments thereof, or polynucleotides encoding MrkA polypeptides or immunogenic fragments thereof described herein is useful in subjects exposed to a Klebsiella contaminated device, including, e.g., a ventilator, a catheter, or an intravenous catheter.

In some embodiments, the pharmaceutical composition comprises an amount of a MrkA-binding agent (e.g., an antibody or antigen-binding fragment thereof) that is effective to inhibit growth of the Klebsiella in a subject. In some embodiments, the Klebsiella is K. pneumoniae. K. oxytoca, K. planticola, and/or K. granulomatis. In some embodiments, the Klebsiella is K. pneumoniae, K. oxytoca, and/or K. granulomatis. In some embodiments, the Klebsiella is K. pneumoniae.

In some embodiments, the pharmaceutical composition comprises an amount of a MrkA polypeptide, immunogenic fragment thereof, or polynucleotide encoding a MrkA polypeptide or immunogenic fragment thereof that is effective to elicit an immune response to Klebsiella, e.g., the production of antibodies, in a subject. In some embodiments, the Klebsiella is K. pneumoniae. K. oxytoca, K. planticola, and/or K. granulomatis. In some embodiments, the Klebsiella is K. pneumoniae. K. oxytoca, and/or K. granulomatis. In some embodiments, the Klebsiella is K. pneumoniae.

In some embodiments, the methods of treating, preventing and/or ameliorating a condition associated with a Klebsiella infection comprises contacting a subject infected with a Klebsiella with a pharmaceutical composition comprising a MrkA-binding protein (e.g., an anti-MrkA antibody or antigen-binding fragment thereof), a MrkA polypeptide, an immunogenic fragment thereof, or a polynucleotide encoding a MrkA polypeptide or immunogenic fragment thereof in vivo. In some embodiments, a pharmaceutical composition comprising a MrkA-binding protein, a MrkA polypeptide, an immunogenic fragment thereof, or a polynucleotide encoding a MrkA polypeptide or immunogenic fragment thereof is administered at the same time or shortly after a subject has been exposed to bacteria to prevent infection. In some embodiments, the pharmaceutical composition comprising a MrkA-binding protein is administered as a therapeutic after infection.

In certain embodiments, the method of treating, preventing, and/or ameliorating Klebsiella infections comprises administering to a subject a pharmaceutical composition comprising a MrkA-binding agent (e.g., an anti-MrkA antibody or antigen-binding fragment thereof), a MrkA polypeptide, an immunogenic fragment thereof, or a polynucleotide encoding a MrkA polypeptide or immunogenic fragment thereof. In certain embodiments, the subject is a human. In some embodiments, the pharmaceutical composition comprising a MrkA-binding protein (e.g., an anti-MrkA antibody or antigen-binding fragment thereof), a MrkA polypeptide, an immunogenic fragment thereof, or a polynucleotide encoding a MrkA polypeptide or immunogenic fragment thereof is administered before the subject is infected with Klebsiella. In some embodiments, the pharmaceutical composition comprising a MrkA-binding protein (e.g., an-anti MrkA antibody or antigen-binding fragment thereof), a MrkA polypeptide, an immunogenic fragment thereof, or a polynucleotide encoding a MrkA polypeptide or immunogenic fragment thereof is administered after the subject is infected with a Klebsiella.

In certain embodiments, the pharmaceutical composition comprising a MrkA-binding agent (e.g., an anti-MrkA antibody or antigen-binding fragment thereof), a MrkA polypeptide, an immunogenic fragment thereof, or a polynucleotide encoding a MrkA polypeptide or immunogenic fragment thereof is administered to a subject on a ventilator. In certain embodiments, the subject has a catheter (e.g., a urinary catheter or an intravenous catheter). In certain embodiments, the subject is receiving antibiotics.

In certain embodiments, a pharmaceutical composition comprising a MrkA-binding agent (e.g., an anti-MrkA antibody or antigen-binding fragment thereof), a MrkA polypeptide, an immunogenic fragment thereof, or a polynucleotide encoding a MrkA polypeptide or immunogenic fragment thereof is for the treatment or prevention of a nosocomial Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, a polynucleotide encoding a MrkA polypeptide or immunogenic fragment thereof is for the treatment or prevention of an opportunistic Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a Klebsiella infection following an organ transplant.

In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a cephalosporin resistant Klebsiella infection. In certain embodiments, a pharmaceutical composition comprising a MrkA-binding agent (e.g., an anti-MrkA antibody or antigen-binding fragment thereof) MrkA polypeptide, an immunogenic fragment thereof, or a polynucleotide encoding a MrkA polypeptide or immunogenic fragment thereof is for the treatment or prevention of an aminoglycoside resistant Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a quinolone resistant Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a carbapenem resistant Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a cephalosporin, aminoglycoside, quinolone, and carbapenem resistant Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of infection with Klebsiella that produce extended spectrum beta-lactamase (ESBL). In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of a cephalosporin, aminoglycoside, and quinolone resistant Klebsiella infection. In certain embodiments, a pharmaceutical composition comprising a MrkA-binding agent (e.g., an anti-MrkA antibody or antigen-binding fragment thereof), MrkA polypeptide, an immunogenic fragment thereof, or a polynucleotide encoding a MrkA polypeptide or immunogenic fragment thereof is for the treatment or prevention of an infection with Klebsiella that produce carbapenemase.

For the treatment, prevention and/or amelioration of a condition associated with a Klebsiella infection, the appropriate dosage of a pharmaceutical composition, antibody, anti-MrkA binding agent, MrkA polypeptide, immunogenic fragment thereof, or polynucleotide encoding a MrkA polypeptide or immunogenic fragment thereof described herein depends on the type of condition, the severity and course of the condition, the responsiveness of the condition, whether the pharmaceutical composition, antibody, anti-MrkA binding agent, MrkA polypeptide, immunogenic fragment thereof, or polynucleotide encoding a MrkA polypeptide or immunogenic fragment thereof is administered for therapeutic or preventative purposes, previous therapy, patient's clinical history, and so on all at the discretion of the treating physician. The pharmaceutical composition, antibody, anti-MrkA binding agent. MrkA polypeptide, immunogenic fragment thereof, or polynucleotide encoding a MrkA polypeptide or immunogenic fragment thereof can be administered one time or over a series of treatments lasting from several days to several months, or until a cure is effected or a diminution of the condition is achieved. Optimal dosing schedules can be calculated from measurements of drug accumulation in the body of the patient and will vary depending on the relative potency of an individual antibody or agent. The administering physician can easily determine optimum dosages, dosing methodologies and repetition rates.

As provided herein, MrkA, an immunogenic fragment thereof, or a polynucleotide encoding a MrkA polypeptide or immunogenic fragment thereof can be administered to a subject to protect from infection with Klebsiella, e.g., by eliciting antibodies to a protective MrkA antigen. In further aspects, an immunogenic composition comprising MrkA, an immunogenic fragment thereof, or a polynucleotide encoding a MrkA polypeptide or immunogenic fragment thereof can be utilized to produce antibodies to diagnose Klebsiella infections, or to produce vaccines for prophylaxis and/or treatment of such Klebsiella infections as well as booster vaccines to maintain a high titer of antibodies against the immunogen(s) of the immunogenic composition.

In some embodiments, the MrkA or immunogenic fragment thereof is K. pneumoniae MrkA or an immunogenic fragment thereof. In some embodiments, the MrkA or immunogenic fragment thereof is K. pneumoniae MrkA. In some embodiments, the MrkA or immunogenic fragment thereof comprises the sequence set forth in SEQ ID NO: 17. In some embodiments, the MrkA or immunogenic fragment thereof is monomeric. In some embodiments, the MrkA or immunogenic fragment thereof is oligomeric.

In some embodiments, the MrkA or immunogenic fragment thereof comprises a sequence at least 75% identical to the sequence set forth in SEQ ID NO: 17. In some embodiments, the MrkA or immunogenic fragment thereof comprises a sequence at least 80% identical to the sequence set forth in SEQ ID NO: 17. In some embodiments, the MrkA or immunogenic fragment thereof comprises a sequence at least 85% identical to the sequence set forth in SEQ ID NO: 17. In some embodiments, the MrkA or immunogenic fragment thereof comprises a sequence at least 90% identical to the sequence set forth in SEQ ID NO: 17. In some embodiments, the MrkA or immunogenic fragment thereof comprises a sequence at least 95% identical to the sequence set forth in SEQ ID NO: 17. In some embodiments, the MrkA or immunogenic fragment thereof comprises a sequence at least 96% identical to the sequence set forth in SEQ ID NO: 17. In some embodiments, the MrkA or immunogenic fragment thereof comprises a sequence at least 97% identical to the sequence set forth in SEQ ID NO: 17. In some embodiments, the MrkA or immunogenic fragment thereof comprises a sequence at least 98% identical to the sequence set forth in SEQ ID NO: 17. In some embodiments, the MrkA or immunogenic fragment thereof comprises a sequence at least 99% identical to the sequence set forth in SEQ ID NO: 17.

In some embodiments, the MrkA or immunogenic fragment thereof comprises amino acids 1-40 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 1-50 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 1-100 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 1-150 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 1-175 of SEQ ID NO: 17 or a sequence at least 75%, 80%, 85%, 90%, 95%, 96%, 97%, 98%, or 99% identical thereto.

In some embodiments, the MrkA or immunogenic fragment thereof comprises amino acids 171-202 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 150-202 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 100-202 of SEQ ID NO: 17 or a sequence at least 75%, 80%, 85%, 90%, 95%, 96%, 97%, 98%, or 99% identical thereto. In some embodiments, the MrkA or immunogenic fragment thereof comprises amino acids 50-202 of SEQ ID NO: 17 or a sequence at least 75%, 80%, 85%, 90%, 95%, 96%, 97%, 98%, or 99% identical thereto.

In some embodiments, the MrkA or immunogenic fragment thereof comprises amino acids 1-40 and 171-202 of SEQ ID NO:17 or a sequence at least 75%, 80%, 85%, 90%, 95%, 96%, 97%, 98%, or 99% identical thereto.

In some embodiments, the MrkA or immunogenic fragment thereof comprises the sequence set forth in SEQ ID NO: 19. In some embodiments, the MrkA or immunogenic fragment thereof comprises a sequence at least 75% identical to the sequence set forth in SEQ ID NO: 19. In some embodiments, the MrkA or immunogenic fragment thereof comprises a sequence at least 80% identical to the sequence set forth in SEQ ID NO: 19. In some embodiments, the MrkA or immunogenic fragment thereof comprises a sequence at least 85% identical to the sequence set forth in SEQ ID NO: 19. In some embodiments, the MrkA or immunogenic fragment thereof comprises a sequence at least 90% identical to the sequence set forth in SEQ ID NO: 19. In some embodiments, the MrkA or immunogenic fragment thereof comprises a sequence at least 95% identical to the sequence set forth in SEQ ID NO: 19. In some embodiments, the MrkA or immunogenic fragment thereof comprises a sequence at least 96% identical to the sequence set forth in SEQ ID NO: 19. In some embodiments, the MrkA or immunogenic fragment thereof comprises a sequence at least 97% identical to the sequence set forth in SEQ ID NO: 19. In some embodiments, the MrkA or immunogenic fragment thereof comprises a sequence at least 98% identical to the sequence set forth in SEQ ID NO: 19. In some embodiments, the MrkA or immunogenic fragment thereof comprises a sequence at least 99% identical to the sequence set forth in SEQ ID NO:19.

In some embodiments, the MrkA or immunogenic fragment thereof comprises amino acids 1-42 of SEQ ID NO: 19 or a sequence at least 75%, 80%, 85%, 90%, 95%, 96%, 97%, 98%, or 999% identical thereto. In some embodiments, the MrkA or immunogenic fragment thereof comprises amino acids 1-50 of SEQ ID NO: 19 or a sequence at least 75%, 80%, 85%, 90%, 95%, 96%, 97%, 98%, or 99% identical thereto. In some embodiments, the MrkA or immunogenic fragment thereof comprises amino acids 1-100 of SEQ ID NO: 19 or a sequence at least 75%, 80⁰%, 85%, 90%, 95%, 96%, 97%, 98%, or 99% identical thereto. In some embodiments, the MrkA or immunogenic fragment thereof comprises amino acids 1-150 of SEQ ID NO: 19 or a sequence at least 75%, 80%, 85%, 90%, 95%, 96%, 97%, 98%, or 99% identical thereto. In some embodiments, the MrkA or immunogenic fragment thereof comprises amino acids 1-175 of SEQ ID NO: 19 or a sequence at least 75%, 80%, 85%, 90%, 95%, 96%, 97%, 98%, or 99% identical thereto.

In some embodiments, the MrkA or immunogenic fragment thereof comprises amino acids 173-204 of SEQ ID NO: 19 or a sequence at least 75%, 80%, 85%, 90%, 95%, 96%, 97%, 98%, or 99% identical thereto. In some embodiments, the MrkA or immunogenic fragment thereof comprises amino acids 150-204 of SEQ ID NO: 19 or a sequence at least 75%, 80%, 85%, 90%, 95%, 96%, 97%, 98%, or 99% identical thereto. In some embodiments, the MrkA or immunogenic fragment thereof comprises amino acids 100-204 of SEQ ID NO: 19 or a sequence at least 75%, 80%, 85%, 90%, 95%, 96%, 97%, 98%, or 99% identical thereto. In some embodiments, the MrkA or immunogenic fragment thereof comprises amino acids 50-204 of SEQ ID NO: 19 or a sequence at least 75%, 80%, 85%, 90%, 95%, 96%, 97%, 98%, or 99% identical thereto.

Vaccines can be prepared as injectables, either as liquid solutions or suspensions. Vaccines in an oil base are also well known such as for inhaling. Solid forms which are dissolved or suspended prior to use can also be formulated. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. Vaccine compositions can comprise substances to stabilize pH, or to function as adjuvants, wetting agents, or emulsifying agents, which can serve to improve the effectiveness of the vaccine. In some embodiments, a vaccine comprises one or more adjuvants.

Vaccine administration is generally by conventional routes, for instance, intravenous, subcutaneous, intraperitoneal, or mucosal routes. The administration can be by parenteral injection, for example, a subcutaneous or intramuscular injection.

The vaccine may be given in a single dose schedule, or optionally in a multiple dose schedule. The amount of vaccine sufficient to confer immunity to Klebsiella is determined by methods well known to those skilled in the art. This quantity will be determined based upon the characteristics of the vaccine recipient, including considerations of age, sex, and general physical condition, and the level of immunity required.

### IV. Methods of Use

The MrkA-binding agents (including, e.g., anti-MrkA antibodies and antigen-binding fragments thereof), MrkA polypeptides, immunogenic fragments thereof, and polynucleotides encoding MrkA polypeptides or immunogenic fragments thereof described herein are useful in a variety of applications including, but not limited to, pneumonia, urinary tract infection, septicemia, neonatal septicemia, diarrhea, soft tissue infection, infection following an organ transplant, surgery infection, wound infection, lung infection, pyogenic liver abscesses (PLA), endophthalmitis, meningitis, necrotizing meningitis, ankylosing spondylitis, and spondyloarthropathies. In some embodiments, the MrkA-binding agents (including antibodies and antigen-binding fragments thereof), MrkA polypeptides, immunogenic fragments thereof, and polynucleotides encoding MrkA polypeptides or immunogenic fragments thereof described herein are useful in nosocomial infections, opportunistic infections, infections following organ transplants, and other conditions associated with a Klebsiella infection (e.g. infection with K. pneumoniae, K. oxytoca, K. planticola, and/or K. granulomatis). In some embodiments, the MrkA-binding agents, MrkA polypeptides, immunogenic fragments thereof, and polynucleotides encoding MrkA polypeptides or immunogenic fragments thereof are useful in subjects exposed to a Klebsiella contaminated device, including, e.g., a ventilator, a catheter, or an intravenous catheter.

In some embodiments, the disclosure provides methods of treating, preventing and/or ameliorating a condition associated with a Klebsiella infection comprising administering an effective amount of a MrkA-binding agent (e.g., an anti-MrkA antibody or antigen-binding fragment thereof), MrkA polypeptide, immunogenic fragment thereof, or polynucleotide encoding a MrkA polypeptide or immunogenic fragment thereof to a subject. In some embodiments, the amount is effective to inhibit growth of the Klebsiella in the subject. In some embodiments, the Klebsiella is K. pneumoniae, K. oxytoca, K. planticola, and/or K. granulomatis. In some embodiments, the Klebsiella is K. pneumoniae. K oxytoca, and/or K. granulomatis. In some embodiments, the Klebsiella is K. pneumoniae. In some embodiments, the subject has been exposed to Klebsiella. In some embodiments, Klebsiella has been detected in the subject. In some embodiments, the subject is suspected of being infected with Klebsiella, e.g., based on symptoms.

In some embodiments, the disclosure provides methods of treating, preventing and/or ameliorating a condition associated with a Klebsiella infection comprising administering an amount of a MrkA polypeptide, immunogenic fragment thereof, or polynucleotide encoding a MrkA polypeptide or immunogenic fragment thereof to a subject, wherein the amount is effective to produce an immune response (e.g., the production of antibodies) to Klebsiella in the subject. In some embodiments, the Klebsiella is K. pneumoniae. K. oxytoca, K. planticola, and/or K. granulomatis. In some embodiments, the Klebsiella is K. pneumoniae, K. oxytoca, and/or K. granulomatis. In some embodiments, the Klebsiella is K. pneumoniae. In some embodiments, the subject has been exposed to Klebsiella. In some embodiments, Klebsiella has been detected in the subject. In some embodiments, the subject is suspected of being infected with Klebsiella, e.g., based on symptoms.

In some embodiments, the disclosure further provides methods of inhibiting growth of Klebsiella comprising administering a MrkA-binding agent (e.g., an anti-MrkA antibody or antigen-binding fragment thereof), MrkA polypeptide, immunogenic fragment thereof, or polynucleotide encoding a MrkA polypeptide or immunogenic fragment thereof to a subject. In some embodiments, the Klebsiella is K. pneumoniae, K. oxytoca. K. planticola, and/or K. granulomatis. In some embodiments, the Klebsiella is K. pneumoniae, K. oxytoca, and/or K. granulomatis. In some embodiments, the Klebsiella is K. pneumoniae. In some embodiments, the subject has been exposed to Klebsiella. In some embodiments, Klebsiella has been detected in the subject. In some embodiments, the subject is suspected of being infected with a Klebsiella, e.g., based on symptoms.

In some embodiments, the methods of treating, preventing and/or ameliorating a condition associated with a Klebsiella infection comprises contacting a subject infected with a Klebsiella with the MrkA-binding agent (e.g., an anti-MrkA antibody or antigen-binding fragment thereof), MrkA polypeptide, immunogenic fragment thereof, or polynucleotide encoding a MrkA polypeptide or immunogenic fragment thereof in vivo. In certain embodiments, contacting a cell with a MrkA-binding agent, MrkA polypeptide, immunogenic fragment thereof, or polynucleotide encoding a MrkA polypeptide or immunogenic fragment thereof is undertaken in a subject. For example, MrkA-binding agents, MrkA polypeptides, immunogenic fragments thereof, and polynucleotides encoding a MrkA polypeptides or immunogenic fragments thereof can be administered to a mouse Klebsiella infection model to reduce bacterial burden. In some embodiments, the MrkA-binding agent, MrkA polypeptide, immunogenic fragment thereof, or polynucleotide encoding a MrkA polypeptide or immunogenic fragment thereof is administered before introduction of bacteria to the subject to prevent infections. In some embodiments, the MrkA-binding agent, MrkA polypeptide, immunogenic fragment thereof, or polynucleotide encoding a MrkA polypeptide or immunogenic fragment thereof is administered at the same time or shortly after the subject has been exposed to bacteria to prevent infection. In some embodiments, the MrkA-binding agent, MrkA polypeptide, immunogenic fragment thereof, or polynucleotide encoding a MrkA polypeptide or immunogenic fragment thereof is administered to the subject as a therapeutic after infection.

In certain embodiments, the method of treating, preventing, and/or ameliorating Klebsiella infections comprises administering to a subject an effective amount of a MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof. In certain embodiments, the subject is a human. In some embodiments, the effective amount of a MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof is administered before the subject or patient is infected with Klebsiella. In some embodiments, the effective amount of a MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof is administered after the subject or patient is infected with a Klebsiella.

In certain embodiments, the subject is on a ventilator. In certain embodiments, the subject has a catheter (e.g., a urinary catheter or an intravenous catheter). In certain embodiments, the subject is receiving antibiotics.

In certain embodiments, the Klebsiella infection is a nosocomial infection. In certain embodiments, the Klebsiella infection is an opportunistic infection. In certain embodiments, the Klebsiella infection follows an organ transplant.

In certain embodiments, the Klebsiella is cephalosporin resistant. In certain embodiments, the Klebsiella is aminoglycoside resistant. In certain embodiments, the Klebsiella is quinolone resistant. In certain embodiments, the Klebsiella is carbapenem resistant. In certain embodiments, the Klebsiella is cephalosporin, aminoglycoside, quinolone, and carbapenem resistant. In certain embodiments, the Klebsiella produce extended spectrum beta-lactamase (ESBL). In certain embodiments, the Klebsiella is cephalosporin, aminoglycoside, and quinolone resistant. In certain embodiments, the Klebsiella produce carbapenemase.

In certain embodiments, the method of treating, preventing, and/or ameliorating Klebsiella infections comprises administering to a subject an effective amount of a MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof and an antibiotic. The MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof and the antibiotic can be administered simultaneously or sequentially. The MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof and the antibiotic can be administered in the same pharmaceutical composition. The MrkA-binding protein (e.g., an anti-MrkA antibody or antigen-binding fragment thereof), MrkA polypeptide, immunogenic fragment thereof, or polynucleotide encoding a MrkA polypeptide or immunogenic fragment thereof and the antibiotic can be administered in separate pharmaceutical compositions simultaneously or sequentially. The antibiotic can be, for example, a carbapanem or colistin.

The present disclosure also provides methods of detecting MrkA, e.g., MrkA oligomers. In some embodiments, a method of detecting MrkA or a MrkA oligomer comprises contacting a sample with a MrkA antibody or antigen-binding fragment thereof provided herein and assaying for binding of the antibody or antigen-binding fragment thereof to the sample. Methods of assessing binding are well known in the art.

### V. Kits

A kit comprising an isolated antigen-binding protein (e.g. an anti-MrkA antibody molecule or antigen-binding fragment thereof), MrkA polypeptide, immunogenic fragment thereof, or polynucleotide encoding a MrkA polypeptide or immunogenic fragment thereof according to any aspect or embodiment of the present disclosure is also provided as an aspect of the present disclosure. In a kit, the antigen-binding protein or anti-MrkA antibody, MrkA polypeptide, immunogenic fragment thereof, or polynucleotide encoding a MrkA polypeptide or immunogenic fragment thereof can be labeled to allow its reactivity in a sample to be determined. e.g. as described further below. Components of a kit are generally sterile and in sealed vials or other containers. Kits can be employed in diagnostic analysis or other methods for which antibody molecules are useful. A kit can contain instructions for use of the components in a method, e.g. a method in accordance with the present disclosure. Ancillary materials to assist in or to enable performing such a method may be included within a kit of the disclosure.

The reactivities of antibodies or antigen-binding fragments thereof in a sample can be determined by any appropriate means. Radioimmunoassay (RIA) is one possibility. Radioactive labeled antigen is mixed with unlabeled antigen (the test sample) and allowed to bind to the antibody. Bound antigen is physically separated from unbound antigen and the amount of radioactive antigen bound to the antibody determined. The more antigen there is in the test sample the less radioactive antigen will bind to the antibody. A competitive binding assay can also be used with non-radioactive antigen, using antigen or an analogue linked to a reporter molecule. The reporter molecule can be a fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules can be enzymes which catalyze reactions that develop or change colors or cause changes in electrical properties, for example. They can be molecularly excitable, such that electronic transitions between energy states result in characteristic spectral absorptions or emissions. They can include chemical entities used in conjunction with biosensors. Biotin/avidin or biotin/streptavidin and alkaline phosphatase detection systems can be employed.

The signals generated by individual antibody-reporter conjugates can be used to derive quantifiable absolute or relative data of the relevant antibody binding in samples (normal and test).

The present disclosure also provides the use of an antigen-binding protein as described above for measuring antigen levels in a competition assay, including methods of measuring the level of MrkA in a sample by employing an antigen-binding protein provided by the present disclosure in a competition assay. In some embodiments, the physical separation of bound from unbound antigen is not required. In some embodiments, a reporter molecule is linked to the antigen-binding protein so that a physical or optical change occurs on binding. The reporter molecule can directly or indirectly generate detectable, and preferably measurable, signals. In some embodiments, the linkage of reporter molecules is direct or indirect, or covalent, e.g., via a peptide bond or non-covalent interaction. Linkage via a peptide bond can be as a result of recombinant expression of a gene fusion encoding antibody and reporter molecule.

The present disclosure also provides methods of measuring levels of MrkA directly, by employing an antigen-binding protein according to the disclosure. In some embodiments, these methods utilize a biosensor system.

### VI. Polynucleotides and Host Cells

In further aspects, the present disclosure provides an isolated nucleic acid comprising a nucleic acid sequence encoding an antigen-binding protein, VH domain and/or VL domain, MrkA polypeptide, or immunogenic fragment thereof according to the present disclosure. In some aspects the present disclosure provides methods of making or preparing an antigen-binding protein, a VH domain and/or a VL domain, MrkA polypeptide, or immunogenic fragment thereof described herein, comprising expressing said nucleic acid under conditions to bring about production of said antigen-binding protein, VH domain and/or VL domain, MrkA polypeptide, or immunogenic fragment thereof and, optionally, recovering the antigen-binding protein, VH domain and/or VL domain, MrkA polypeptide, or immunogenic fragment thereof.

A nucleic acid provided by the present disclosure includes DNA and/or RNA. In one aspect, the nucleic acid is cDNA. In one aspect, the present disclosure provides a nucleic acid which codes for a CDR or set of CDRs or VH domain or VL domain or antibody antigen-binding site or antibody molecule, e.g., scFv or IgG1, as described above.

One aspect of the present disclosure provides a nucleic acid, generally isolated, optionally a cDNA, encoding a VH CDR or VL CDR sequence described herein. In some embodiments, the VH CDR is selected from SEQ ID NOs: 1-6 or 29-40. In some embodiments, the VL CDR is selected from SEQ ID NOs: 7-12 or 41-52. A nucleic acid encoding the Kp3, Kp16, clone 1, clone 4, clone 5, or clone 6 set of CDRs, a nucleic acid encoding the Kp3, Kp16, clone 1, clone 4, clone 5, or clone 6 set of HCDRs and a nucleic acid encoding the Kp3, KP16, clone 1, clone 4, clone 5, or clone 6 set of LCDRs are also provided, as are nucleic acids encoding individual CDRs, HCDRs, LCDRs and sets of CDRs, HCDRs, LCDRs as described in Tables 1 and 2. In some embodiments, the nucleic acids of the present disclosure encode a VH and/or VL domain of Kp3. Kp16, clone 1, clone 4, clone 5, or clone 6 as described in Tables 3 and 4.

In some embodiments, the polynucleotide encodes a sequence at least 75% identical to the sequence set forth in SEQ ID NO: 17. In some embodiments, the polynucleotide encodes a sequence at least 80% identical to the sequence set forth in SEQ ID NO: 17. In some embodiments, the polynucleotide encodes a sequence at least 85% identical to the sequence set forth in SEQ ID NO: 17. In some embodiments, the polynucleotide encodes a sequence at least 90% identical to the sequence set forth in SEQ ID NO: 17. In some embodiments, the polynucleotide encodes a sequence at least 95% identical to the sequence set forth in SEQ ID NO: 17. In some embodiments, the polynucleotide encodes a sequence at least 96% identical to the sequence set forth in SEQ ID NO: 17. In some embodiments, the polynucleotide encodes a sequence at least 97% identical to the sequence set forth in SEQ ID NO: 17. In some embodiments, the polynucleotide encodes a sequence at least 98% identical to the sequence set forth in SEQ ID NO: 17. In some embodiments, the polynucleotide encodes a sequence at least 99% identical to the sequence set forth in SEQ ID NO:17.

In some embodiments, the polynucleotide encodes amino acids 1-40 of SEQ ID NO: 17 or a sequence at least 75%, 80%, 85%, 90%, 95%, 96%, 97%, 98%, or 990% identical thereto. In some embodiments, the polynucleotide encodes amino acids 1-50 of SEQ ID NO: 17 or a sequence at least 75%, 80%, 85%, 90%, 95%, 96%, 97%, 98%, or 99% identical thereto. In some embodiments, the polynucleotide encodes amino acids 1-100 of SEQ ID NO: 17 or a sequence at least 75%, 80%, 85%, 90%, 95%, 96%, 97%, 98%, or 99% identical thereto. In some embodiments, the polynucleotide encodes amino acids 1-150 of SEQ ID NO: 17 or a sequence at least 75%, 80%, 85%, 90%, 95%, 96%, 97%, 98%, or 99% identical thereto. In some embodiments, the polynucleotide encodes amino acids 1-175 of SEQ ID NO: 17 or a sequence at least 75%, 80%, 85%, 90%, 95%, 96%, 97%, 98%, or 99% identical thereto.

In some embodiments, the polynucleotide encodes amino acids 171-202 of SEQ ID NO: 17 or a sequence at least 75%, 80%, 85%, 90%, 95%, 96%, 97%, 98%, or 99% identical thereto. In some embodiments, the polynucleotide encodes amino acids 150-202 of SEQ ID NO: 17 or a sequence at least 75%, 80%, 85%, 90%, 95%, 96%, 97%, 98%, or 99% identical thereto. In some embodiments, the polynucleotide encodes amino acids 100-202 of SEQ ID NO: 17 or a sequence at least 75%, 80%, 85%, 90%, 95%, 96%, 97%, 98%, or 99% identical thereto. In some embodiments, the polynucleotide encodes amino acids 50-202 of SEQ ID NO: 17 or a sequence at least 75%, 80%, 85%, 90%, 95%, 96%, 97%, 98%, or 99% identical thereto.

In some embodiments, the polynucleotide encodes amino acids 1-40 and 171-202 of SEQ ID NO: 17 or a sequence at least 75%, 80%, 85%, 90%, 95%, 96%, 97%, 98%, or 99% identical thereto.

In some embodiments, the polynucleotide encodes the sequence set forth in SEQ ID NO: 19. In some embodiments, the polynucleotide encodes a sequence at least 75% identical to the sequence set forth in SEQ ID NO: 19. In some embodiments, the polynucleotide encodes a sequence at least 80% identical to the sequence set forth in SEQ ID NO: 19. In some embodiments, the polynucleotide encodes a sequence at least 85% identical to the sequence set forth in SEQ ID NO: 19. In some embodiments, the polynucleotide encodes a sequence at least 90% identical to the sequence set forth in SEQ ID NO: 19. In some embodiments, the polynucleotide encodes a sequence at least 95% identical to the sequence set forth in SEQ ID NO: 19. In some embodiments, the polynucleotide encodes a sequence at least 96% identical to the sequence set forth in SEQ ID NO: 19. In some embodiments, the polynucleotide encodes a sequence at least 97% identical to the sequence set forth in SEQ ID NO: 19. In some embodiments, the polynucleotide encodes a sequence at least 98% identical to the sequence set forth in SEQ ID NO: 19. In some embodiments, the polynucleotide encodes a sequence at least 99% identical to the sequence set forth in SEQ ID NO:19.

In some embodiments, the polynucleotide encodes amino acids 1-42 of SEQ ID NO: 19 or a sequence at least 75%, 80%, 85%, 90%, 95%, 96%, 97%, 98%, or 99% identical thereto. In some embodiments, the polynucleotide encodes amino acids 1-50 of SEQ ID NO: 19 or a sequence at least 75%, 80%, 85%, 90%, 95%, 96%, 97%, 98%, or 99/identical thereto. In some embodiments, the polynucleotide encodes amino acids 1-100 of SEQ ID NO: 19 or a sequence at least 75%, 80%, 85%, 90%, 95%, 96%, 97%, 98%, or 99% identical thereto. In some embodiments, the polynucleotide encodes amino acids 1-150 of SEQ ID NO: 19 or a sequence at least 75%, 80%, 85%, 90%, 95%, 96%, 97%, 98%, or 99% identical thereto. In some embodiments, the polynucleotide encodes amino acids 1-175 of SEQ ID NO: 19 or a sequence at least 75%, 80%, 85%, 90%, 95%, 96%, 97%, 98%, or 99% identical thereto.

In some embodiments, the polynucleotide encodes amino acids 173-204 of SEQ ID NO: 19 or a sequence at least 75%, 80%, 85%, 90%, 95%, 96%, 97%, 98%, or 99% identical thereto. In some embodiments, the polynucleotide encodes amino acids 150-204 of SEQ ID NO: 19 or a sequence at least 75%, 80%, 85%, 90%, 95%, 96%, 97%, 98%, or 99% identical thereto. In some embodiments, the polynucleotide encodes amino acids 100-204 of SEQ ID NO: 19 or a sequence at least 75%, 80%, 85%, 90%, 95%, 96%, 97%, 98%, or 99% identical thereto. In some embodiments, the polynucleotide encodes amino acids 50-204 of SEQ ID NO: 19 or a sequence at least 75%, 80%, 85%, 90%, 95%, 96%, 97%, 98%, or 99% identical thereto.

The present disclosure provides an isolated polynucleotide or cDNA molecule sufficient for use as a hybridization probe, PCR primer or sequencing primer that is a fragment of a nucleic acid molecule disclosed herein or its complement. The nucleic acid molecule can, for example, be operably linked to a control sequence.

The present disclosure also provides constructs in the form of plasmids, vectors, transcription or expression cassettes which comprise at least one polynucleotide as described above.

The present disclosure also provides a recombinant host cell which comprises one or more nucleic acids, plasmids, vectors or as described above. A nucleic acid encoding any CDR or set of CDRs or VH domain or VL domain or antibody antigen-binding site, antibody molecule, e.g. scFv or IgG1 as provided (see. e.g. Tables 1-4), MrkA polypeptide, or immunogenic fragment thereof, itself forms an aspect of the present disclosure, as does a method of production of the encoded product, which method comprises expression from the nucleic acid encoding the product (e.g. the antigen binding protein disclosed herein). Expression can conveniently be achieved by culturing under appropriate conditions recombinant host cells containing a nucleic acid described herein. Following production by expression a CDR, set of CDRs, VH or VL domain, an antigen-binding protein, MrkA polypeptide, or immunogenic fragment thereof can be isolated and/or purified using any suitable technique.

In some instances, the host cell is a mammalian host cell, such as a NS0 murine myeloma cell, a PER.C6® human cell, or a Chinese hamster ovary (CHO) cell.

Antigen-binding proteins, VH and/or VL domains, MrkA polypeptides, immunogenic fragments thereof, and encoding nucleic acid molecules and vectors can be isolated and/or purified, e.g. from their natural environment, in substantially pure or homogeneous form, or, in the case of nucleic acid, free or substantially free of nucleic acid or genes of origin other than the sequence encoding a polypeptide with the required function. Nucleic acids according to the present disclosure may comprise DNA or RNA and can be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, plant cells, yeast and baculovirus systems and transgenic plants and animals. Mammalian cell lines available in the art for expression of a heterologous polypeptide include Chinese hamster ovary (CHO) cells, HeLa cells, baby hamster kidney cells, NS0 mouse melanoma cells. YB20 rat myeloma cells, human embryonic kidney cells, human embryonic retina cells and many others. A common bacterial host is E. coli.

The expression of antibodies and antibody fragments in prokaryotic cells such as E. coli is well established in the art. For a review, see for example Plückthun, A. Bio/Technology 9: 545-551 (1991). Expression in eukarvotic cells in culture is also available to those skilled in the art as an option for production of an antigen-binding protein for example Chadd H E and Chamow S M (2001) 110 Current Opinion in Biotechnology 12: 188-194, Andersen D C and Krummen L (2002) Current Opinion in Biotechnology 13: 117, Larrick J W and Thomas D W (2001) Current opinion in Biotechnology 12:411-418.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acids, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1988, Short Protocols in Molecular Biology: A Compendium ofMethods from Current Protocols in Molecular Biology, Ausubel et al. eds., John Wiley & Sons, 4^{th} edition 1999. The disclosures of Sambrook et al. and Ausubel et al. (both) are incorporated herein by reference.

Thus, a further aspect of the present disclosure provides a host cell containing nucleic acid as disclosed herein. For example, the disclosure provides a host cell transformed with nucleic acid comprising a nucleotide sequence encoding an antigen-binding protein of the present disclosure or antibody CDR, set of CDRs, VH and/or VL domain of an antigen-binding protein, MrkA polypeptide, or immunogenic fragment thereof of the present disclosure. In some embodiments, the host cell comprises the expressed antigen-binding protein of the present disclosure or antibody CDR, set of CDRs. VH and/or VL domain of an antigen-binding protein, MrkA polypeptide, or immunogenic fragment thereof of the present disclosure.

Such a host cell can be in vitro and can be in culture. Such a host cell can be an isolated host cell. Such a host cell can be in vivo.

A still further aspect provided herein is a method comprising introducing such nucleic acid into a host cell. The introduction can employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. Introducing nucleic acid in the host cell, in particular a eukaryotic cell can use a viral or a plasmid based system. The plasmid system can be maintained episomally or may be incorporated into the host cell or into an artificial chromosome. Incorporation can be either by random or targeted integration of one or more copies at single or multiple loci. For bacterial cells, suitable techniques may include calcium chloride transformation, electroporation, and transfection using bacteriophage.

The introduction can be followed by causing or allowing expression from the nucleic acid, e.g., by culturing host cells under conditions for expression of the gene.

In one embodiment, the nucleic acid of the present disclosure is integrated into the genome (e.g. chromosome) of the host cell. Integration can be promoted by inclusion of sequences which promote recombination with the genome, in accordance with standard techniques.

The present disclosure also provides a method which comprises using a construct (e.g. plasmid, vector, etc. as described above) in an expression system in order to express an antigen-binding protein or polypeptide as described above.

In another aspect, the disclosure provides a hybridoma producing the antigen-binding protein (e.g. anti-MrkA antibodies or antigen binding fragments thereof) of the disclosure.

A yet further aspect of the disclosure provides a method of production of an antibody binding protein, MrkA polypeptide, or immunogenic fragment thereof of the disclosure, the method including causing expression from encoding nucleic acid. Such a method can comprise culturing host cells under conditions suitable for production of said antigen-binding protein, MrkA polypeptide, or immunogenic fragment thereof.

In some embodiments, the method of production further comprises isolating and/or purifying the antigen binding protein (including antibodies or antigen binding fragments thereof), MrkA polypeptide, or immunogenic fragment thereof produced from the host cell or hybridoma.

### EXAMPLES

In view of the need to identify agents that have protective effective against Klebsiella infections, a novel functionally-based screening assay was used to identify cross-protective targets for the Gram negative bacterium K. pneumoniae. This novel assay identified antibodies capable of inducing opsonophagocytic killing (OPK) and did not focus, at the outset, on any particular target antigen.

Materials and Methods

K. pneumoniae Strain Information

All K. pneumoniae isolates were obtained from America Type Culture Collection (ATCC, Manassas, Va.) or Eurofin collection. The capsule and O-antigen deficient K. pneumoniae 43816 strain (43816ΔcpsBΔWaaL or 43816DM) was constructed through allelic replacement with plasmids containing CpsB and WaaL ORFs and selected in the presence of gentamicin. Gentamicin resistant colonies were picked and expanded. The deletions of the CpsB and WaaL genes were confirmed by PCR analysis. To construct K. pneumoniae strains expressing luciferase (Lux strain), various K. pneumoniae clinical isolates were transformed with a plasmid containing the luciferase reporter gene and gentamicin resistant colonies were selected. Unless stated otherwise, all K. pneumoniae cultures were maintained in 2xYT media at 37° C., supplemented with antibiotics when appropriate.

Phage Panning and Screening

ScFv phage display libraries constructed from healthy donors were used for selection, as described in Vaughan et al., Nature Biotechnology 14:309-14 (1996). For selection, 9×10⁹ K. pneumoniae cells from 43816ΔcpsBΔWaaL were used as the panning antigen for round one, followed by two more rounds of panning on an equal mix of wild type strains 1901 (ATCC BAA-1901) and 1899 (ATCC BAA-1899). For each round, bacterial cells were harvested at mid-log phase and blocked (2xYT+3% dry milk), followed by addition of 1×10¹² blocked phage particles. Cells were then washed seven times by repeated re-suspension in PBS. Bound phage particles were eluted with 0. IN HCl, neutralized with 1M Tris-HCl, pH 8.0, and used to infect TG1 for phage particle amplification and subsequent rounds of panning. TG1 cells infected with third round phage panning output were used to prepare phagemid. ScFv fragments were prepared from the purified phagemids pool and subcloned into a scFv-Fc expression vector for expression and screening in near product format. Clones cross-reactive to 1900, 3556, and MGH78578 isolates were further characterized in the OPK assay.

Isolation of K. pneumonia Specific Hybridomas

Balb/c mice were immunized with 43816ΔcpsBΔWaaL via intraperitoneal (I.P.) route weekly for four weeks followed by a final boost with a mixture of wild type K. pneumoniae clinical isolates (Kp1901 and 1899). At the end of the immunization, lymph node lymphocytes and splenocytes were harvested and fused with P3X myelomas and subjected to selection in 1×HAT culture medium. Supernatants from the resulting hybridomas were then screened for binding to 43816ΔcpsBΔWaaL by whole bacterial ELISA. Positive binders were subjected to the high-throughput OPK assay to select for potentially protective hybridomas against K. pneumoniae.

Anti-K. pneumoniae Whole Bacterial ELISA

The binding of anti-K. pneumoniae antibodies to multiple strains was assessed by ELISA as described in DiGiandomenico, et al., J Exp Med. 209:1273-87 (2012), herein incorporated by reference. Briefly, a single colony of K. pneumoniae was inoculated into 2xYT media until the culture reached log phase. Bacteria were coated onto 384-well plates (Nunc MaxiSorp) overnight at 4° C. A set of plates were coated with similarly prepared culture of Acinetobactor pitti 19004 (ATCC 19004) as negative controls. After blocking with PBS supplemented with 4% BSA (PBS-B), the coated plates were incubated with anti-K. pneumoniae antibodies for 1 h. The plates were then washed with PBS-T (PBS+0.1% Tween 20) before HRP-conjugated secondary antibody was added for 1 h followed by washing and TMB (3, 3′, 5, 5′-Tetrametheylbenzidine) substrate addition. Color development was stopped by adding 0.1 N HCL, and the absorbance at 450 nm was measured by microplate reader (Molecular Devices). The data was plotted with Prism software.

High Throughput Opsonophagocytic Killing (OPK) Assay

OPK assays were performed based on the procedure described in DiGiandomenico, et al., J Exp Med, 209:1273-87 (2012) with modifications. Briefly, log phase culture of luciferase carrying K. pneumoniae strains (Lux) were diluted to ˜2×10⁶ cells/ml. Four components were mixed together in 384-well plates for OPK assays: bacteria, diluted baby rabbit serum (Cedarlane, 1:10), differentiated HL-60 cells, and antibodies. The mixture was incubated at 37° C. for two hours with shaking (250 rpm). The relative light units (RLUs) were then measured using an Envision Multilabel plate reader (Perkin Elmer). The percentage of killing was determined by comparing RLU derived from assays with anti-K. pneumoniae mAbs and a negative control mAb.

Confocal Microscopy

K. pneumoniae 43816 was grown overnight in 2xYT culture medium at 37° C. Fluorescent labeling was achieved by incubating bacteria with the MrkA specific monoclonal antibody Kp3, followed by Alexa 488 labeled anti-human IgG secondary antibody (Invitrogen). Bacteria were then fixed with 4% neutral buffer formalin and mounted on a cover slip. Confocal microscopy was performed with a Leica TCS SP5 confocal system consisting of a Leica DMI6000 B inverted microscope (Leica Microsystems). Images were analyzed using the LAS AF version 2.2.1 Leica Application Suite software (Leica Microsystems).

Immunoprecipitation from Klebsiella pneumoniae Lysate

K. pneumoniae overnight culture was collected by centrifugation, and the cell pellet was re-suspended in 3 ml of B-PER (Thermo Scientific) buffer supplemented with protease inhibitor cocktail and DNaseI (2 μl/ml at 200 U/μl). After incubating at room temperature for 40 min, the supernatant was collected through centrifugation at top speed in a table top Eppendorf centrifuge (14,000 rpm/min) for 20 min at 4° C. The cleared lysate was mixed with 40 μl of protein AG beads (Pierce, #20422) and incubated at 4° C. for 2 hours. The lysate was collected by centrifugation again at top speed (14,000 rpm/min) for 15 min at 4° C. The cleared lysate was moved to a new Eppendorf tube containing 15 μl of protein A/G beads (prewashed with B-PER), 6 μg of immunoprecipitation antibody, incubated on a rotator for 3 hours at 4° C. The beads were then collected by spinning at 10,000 rpm, 1 min at 4° C. followed by three washes with ice cold B-PER buffer. Immunoprecipated samples were then re-suspended in SDS-PAGE buffer and loaded directly onto a SDS-PAGE gel (4-12% gradient gel Novex). Half of the sample was loaded on one gel for blue stain (Invitrogen) and subsequent mass spec sample preparation; the other half was loaded to a second gel for Western blot analysis.

LC-MS Identification of Immunoprecipitation Products

Bands of interest were excised, de-stained and washed, followed by in-gel reduction with dithiothreitol (DTT) and alkylation with iodoacetamide in the dark. Proteins were digested in-gel with trypsin at 37° C. followed by extraction of the digested peptides. The trypsin digested sample was analyzed by on-line nano-LC-MS, using methods similar to the protocol provided in Aboulaich et al., Biotechnol. Prog. 30: 1114-1124 (2014), herein incorporated by reference. The LC separation of peptides was performed on a nano-ACQUITY UPLC® (Waters) system equipped with a 180 μm i.d.×20 mm length C18 Symmetry trap column and a 100 μm×100 mm C18 (Waters) reversed phase column operated at a flow rate of 400 nL/min (Buffer-A: 0.1% formic acid; buffer-B: 0.1% formic acid in acetonitrile) (see Heidbrink Thomspon et al, Rapid Communications in Mass Spectometry 28: 855-60 (2014)). Each sample was injected onto the trap column using 1% buffer B. Peptides were eluted over 60 minutes. After the LC separation, the eluted peptides were analyzed on-line using an LTQ-Orbitrap (top six MS/MS method) mass spectrometer (Thermo Fisher Scientific) in data dependent mode using collisionally induced dissociation (CID) for MS/MS. The identity of each protein was determined by the Proteome Discoverer v. 1.3 software equipped with Sequest and Mascot nodes (Aboulaich et al., Biotechnol. Prog. 30: 1114-1124 (2014)) by searching mass spectral data against a K. pneumoniae protein sequence database (Uniprot). The database also contained a human IgG₁ protein sequence. A minimum of two medium or high confidence (determined in the Peptide Validator node of Proteome Discoverer software) peptides per protein were required to positively identify each protein.

Recombinant MrkA Protein Expression

The MrkA-his tag open reading frame (ORFs) was synthesized, cloned into the expression vector pACYC-duet-1 (EMD Millipore), and transformed into E. coli BL21 (DE3) cells. Chloramphenicol-resistant colonies were picked and expanded in LB media containing 150 μg/ml of chloramphenicol. Once the OD (600 nm) reached 0.4, 1 mM IPTG was added to the culture to induce the expression of MrkA-his at 37° C. for 4 hours. Bacteria were lysed with B-PER, and the presence of MrkA-his was examined by Western blot using anti-his or MrkA specific mAbs as described herein.

In Vitro Transcription and Translation of MrkA Protein

The DNA templates of MrkA for in vitro expression were amplified by PCR. The template includes a T7 promoter at the 5′, a c-Myc tag and T7 terminator at 3′ of MrkA ORF. 250 ng of DNA templates were added to the PURExpress in vitro protein system (NEB E6800) with or without Disulfide Bond Enhancer (NEB E6820S) in 25 itl of reaction mixture, and the reaction mixes were incubated at 37° C. for 2 hours. The synthesized proteins were analyzed by western blot using anti-c-Myc and MrkA specific mAb as described herein.

Bacterial Infection Models

C56/BL6 mice were received from Jackson laboratories and maintained in a special pathogen free facility. All animal experiments were conducted in accordance with IACUC protocol and guidance. K. pneumoniae strains were grown on agar plates overnight and diluted in saline at proper concentration. The inoculum titer was determined by plating serial dilution of bacteria onto agar plates prior to and post challenge. Antibodies and controls were administered 24 hours prior to bacterial infection. For organ burden models, C57/bl6 mice were inoculated with 1e7 CFU bacteria in 50 μl saline intranasally to induce pneumonia. The lung bacterial burden was measured by plating lung homogenates onto agar plates to determine CFU 24 hours post infection. In acute pneumonia models, C57/bl6 mice were inoculated intranasally with 5e3 CFU or 1e8 CFU of K. pneumoniae 43816 strain (O1:K2) or K. pneumoniae 985048 strain, respectively. Kp3 and human IgG1 control antibody were given one day prior to bacterial challenge. Mouse survival was monitored daily until up to day 8. Combined survival data of three experiments were plotted in Prism.

Statistical Analysis

All statistical analysis was performed in GraphPad Prism version 6. For comparing bacterial burden, Kp3 treated animals were compared with human isotype control antibody treated animals by unpaired t test. Survival results were plotted as Kaplan-Meier curves and analyzed as Log-rank (Mental-Cox) tests.

### Example 1: Phage Panning Against Live K. pneumoniae

Human scFv libraries derived from healthy donors (Vaughan et al., Nature Biotechnology 14: 309-14 (1996)) were used to select for K. pneumoniae specific antibodies. This process was designed to select for functionally relevant targets instead of using specific antigens. Due to the highly variable structures of K. pneumoniae capsule polysaccharides and O-antigens, a capsule and O-antigen deleted mutant strain 43816DM (43816ΔcpsBΔWaaL) was generated to drive the selection process toward more conserved surface antigens. The first round of affinity selective panning was performed on 43816DM, followed by two more rounds of panning on a mixture of wild-type isolates (1901 and 1899). More than a hundred-fold enrichment was observed from output titers over three rounds of panning.

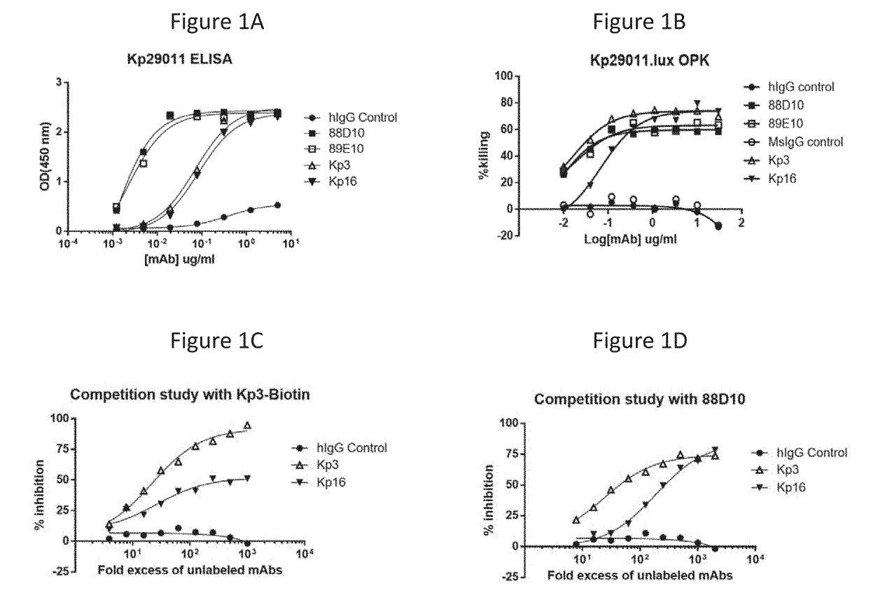

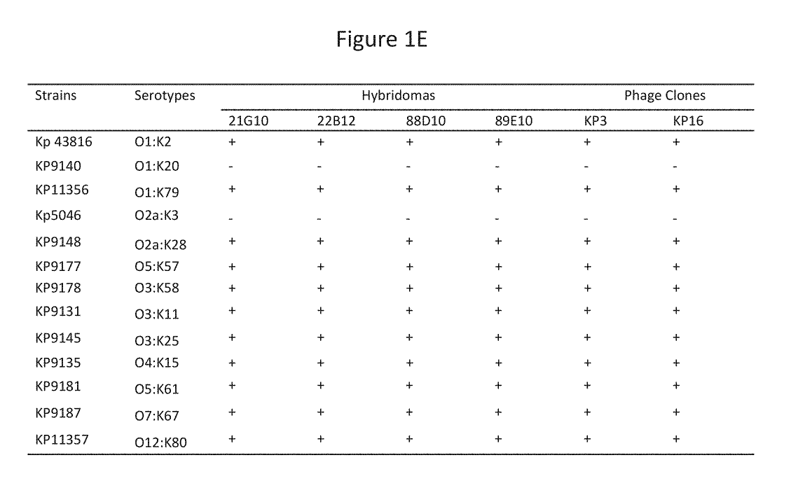

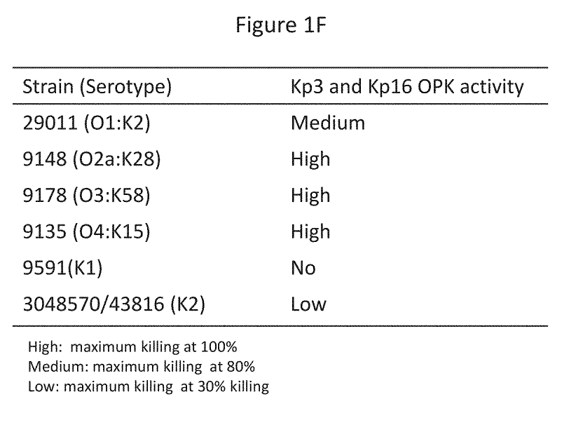

The phage libraries used in this study were single chain fragment variable (scFv) libraries. Through the scFv format is adequate for specific binding based preliminary screenings, it is not suitable for functional screening formats such as OPK because OPK relies on effector function mediated through the Fc fragment. Thus, the third round panning output was batch-converted into scFv-Fc format. This platform allows for scFv-Fc expression in both bacterial and mammalian hosts, which is suitable for both high throughput and functional screening needs. The scFV-Fc clones were expressed in bacteria, and the resulting supernatants were tested for binding to three live K. pneumoniae wild type strains. A total of 3520 scFv-Fc clones were screened, and more than 400 clones displayed specific binding to all three K. pneumoniae isolates. Non-specific binders were excluded by using an irrelevant bacterium as a control during ELISA screens. Sequencing revealed two dominant phage derived clones, Kp3 and Kp16. These were expressed in scFv-Fc format in mammalian cells and tested for OPK activity. After reformatting to IgG1, they retained strong binding to Kp29011 in whole bacterial ELISA (FIG. 1A), displayed potent OPK activity (FIG. 1B) and demonstrated binding to the majority of isolates with different capsule and O-antigen serotypes (FIG. 1E). Kp3 and Kp16 also showed OPK activities against a panel of K. pneumoniae of different serotypes (FIG. 1F). Further testing with an expanded spectrum of seven hundred recent K. pneumoniae clinical isolates, Kp3 bound to more than 62% of the strains, with majority of them being multi-drug resistant isolates. A list of representative K. pneumoniae clinical isolates recognized by Kp3 is shown in Table 5.

TABLE 5
Kp3 binding to multi-drug resistant Klebsiella pneumoniae clinical isolates

| | | IHMA | Body | |
|---|---|---|---|---|
| Region | Country | Number | Location | Facility Name |
| Europe | Italy | 845670 | Respiratory: Endotracheal aspirate | Pediatric ICU |
| Europe | Italy | 845728 | Respiratory: Endotracheal aspirate | Medicine ICU |
| Europe | Portugal | 845904 | Respiratory: Sputum | Medicine ICU |
| Europe | Portugal | 845927 | INT: Wound | Emergency Room |
| Latin | Argentina | 847379 | INT: Skin Ulcer | Medicine ICU |
| America | | | | |
| Middle | Israel | 849156 | Bodily Fluids: Peritoneal | Medicine General |
| East | | | | |
| Middle | Israel | 849584 | INT: Abscess | Pediatric ICU |
| East | | | | |
| Middle | Israel | 849626 | INT: Wound | Medicine General |
| East | | | | |
| Europe | Romania | 850438 | INT: Wound | Surgery General |
| Latin | Chile | 866937 | INT: Wound | Other |
| America | | | | |
| Middle | Israel | 869311 | Respiratory: Bronchial brushing | Medicine ICU |
| East | | | | |
| Europe | Russia | 874876 | Respiratory: Sputum | Pediatric ICU |
| Europe | Italy | 875928 | Respiratory: Endotracheal aspirate | Medicine ICU |
| Latin | Brazil | 900678 | Respiratory: Endotracheal aspirate | Medicine ICU |
| America | | | | |
| Europe | Portugal | 938176 | Respiratory: Sputum | Medicine General |
| Europe | Italy | 946900 | Respiratory: Bronchial brushing | Surgery General |
| Latin | Colombia | 960417 | Respiratory: Bronchoalveolar lavage | Medicine General |
| America | | | | |
| North | United | 961842 | Respiratory: Bronchoalveolar lavage | Medicine ICU |
| America | States | | | |
| North | United | 977784 | Respiratory: Endotracheal aspirate | Other |
| America | States | | | |
| North | United | 979288 | Respiratory: Sputum | Surgery General |
| America | States | | | |
| North | United | 979290 | Respiratory: Sputum | Medicine ICU |
| America | States | | | |
| Latin | Venezuela | 984342 | Respiratory: Endotracheal aspirate | Medicine ICU |
| America | | | | |
| Europe | Poland | 985048 | INT: Wound | Surgery General |
| Latin | Brazil | 991499 | Respiratory: Endotracheal aspirate | Medicine ICU |
| America | | | | |
| Latin | Brazil | 991947 | Respiratory: Bronchoalveolar lavage | Surgery General |
| America | | | | |
| Middle | Israel | 994038 | Respiratory: Endotracheal aspirate | Medicine ICU |
| East | | | | |
| Middle | Israel | 994039 | Respiratory: Endotracheal aspirate | Medicine General |
| East | | | | |
| Asia | China | 996004 | Respiratory: Sputum | None Given |
| Asia | China | 1032915 | Respiratory: Sputum | Medicine General |
| Africa | Kenya | 1046198 | Respiratory: Other | Medicine ICU |
| Europe | Russia | 1049214 | Respiratory: Bronchoalveolar lavage | Surgery General |
| Europe | Russia | 1049391 | Respiratory: Bronchoalveolar lavage | Surgery ICU |
| Europe | Russia | 1049474 | Respiratory: Bronchoalveolar lavage | Surgery General |
| North | United | 1072280 | Respiratory: Bronchoalveolar lavage | Surgery ICU |
| America | States | | | |
| Latin | Venezuela | 1073570 | Respiratory: Endotracheal aspirate | None Given |
| America | | | | |
| Europe | Spain | 1073956 | Respiratory: Bronchial brushing | Medicine ICU |
| Europe | Spain | 1073967 | CVS: Blood | Medicine General |
| South | Philippines | 1079540 | CVS: Blood | Pediatric ICU |
| Pacific | | | | |
| South | Philippines | 1079544 | Respiratory: Endotracheal aspirate | Medicine ICU |
| Pacific | | | | |
| Asia | Thailand | 1082632 | INT: Wound | Surgery General |
| Asia | Korea, | 1085601 | Respiratory: Sputum | Medicine General |
| | South | | | |
| Africa | South | 1088166 | Respiratory: Endotracheal aspirate | Medicine ICU |
| | Africa | | | |
| Europe | Belgium | 1089847 | INT: Wound | Medicine ICU |
| Africa | South | 1093894 | Bodily Fluids: Peritoneal | General |
| | Africa | | | Unspecified ICU |
| Latin | Argentina | 1093960 | Respiratory: Bronchoalveolar lavage | Medicine ICU |
| America | | | | |
| Latin | Argentina | 1093955 | Respiratory: Bronchoalveolar lavage | Medicine ICU |
| America | | | | |
| Latin | Argentina | 1093975 | Respiratory: Bronchoalveolar lavage | Medicine ICU |
| America | | | | |
| Latin | Argentina | 1093976 | Respiratory: Bronchoalveolar lavage | Medicine ICU |
| America | | | | |
| North | United | 1094435 | INT: Wound | Medicine General |
| America | States | | | |
| North | United | 1103982 | INT: Wound | Medicine ICU |
| America | States | | | |
| Middle | Kuwait | 1104304 | Respiratory: Endotracheal aspirate | General |
| East | | | | Unspecified ICU |
| Europe | Greece | 1104866 | Bodily Fluids: Peritoneal | Medicine General |
| North | United | 1105534 | Respiratory: Bronchoalveolar lavage | Medicine General |
| America | States | | | |
| Africa | Kenya | 1106510 | CVS: Blood | Surgery ICU |
| Latin | Colombia | 1109216 | Bodily Fluids: Peritoneal | Surgery General |
| America | | | | |
| Europe | Czech | 1120042 | Respiratory: Sputum | Medicine ICU |
| | Republic | | | |
| Europe | Belgium | 1130776 | Respiratory: Endotracheal aspirate | Surgery ICU |
| Latin | Chile | 1131115 | CVS: Blood | Medicine General |
| America | | | | |
| Latin | Chile | 1131124 | CVS: Blood | Medicine ICU |
| America | | | | |
| Europe | Italy | 1137983 | GI: Abscess | Surgery General |
| Europe | Italy | 1137984 | Respiratory: Bronchial brushing | Medicine ICU |
| Latin | Chile | 1145451 | Respiratory: Endotracheal aspirate | Medicine General |
| America | | | | |
| Latin | Chile | 1145452 | Respiratory: Endotracheal aspirate | Medicine ICU |
| America | | | | |
| North | United | 1147892 | Respiratory: Endotracheal aspirate | Medicine General |
| America | States | | | |
| North | United | 1147894 | Respiratory: Endotracheal aspirate | Medicine ICU |
| America | States | | | |
| Latin | Chile | 847204 | INT: Wound | Surgery General |
| America | | | | |
| Latin | Argentina | 847694 | Unknown | Medicine ICU |
| America | | | | |
| Latin | Argentina | 847747 | Respiratory: Endotracheal aspirate | Medicine ICU |
| America | | | | |
| Middle | Israel | 849585 | INT: Wound | Medicine General |
| East | | | | |
| Middle | Israel | 849624 | INT: Wound | Medicine General |
| East | | | | |
| South | Philippines | 850793 | SSI: Abscess Cavity | Other |
| Pacific | | | | |
| North | United | 863890 | INT: Decubitus | None Given |
| America | States | | | |
| Europe | Italy | 867822 | Bodily Fluids: Peritoneal | Surgery General |
| Europe | Belgium | 869028 | Respiratory: Other | Surgery ICU |
| Europe | Romania | 869918 | Respiratory: Sputum | General |
| | | | | Unspecified ICU |
| Europe | Romania | 869921 | Respiratory: Endotracheal aspirate | General |
| | | | | Unspecified ICU |
| North | United | 873461 | INT: Wound | Surgery ICU |
| America | States | | | |
| Europe | Russia | 874316 | Respiratory: Sputum | General |
| | | | | Unspecified ICU |
| Europe | Russia | 874329 | Respiratory: Other | General |
| | | | | Unspecified ICU |
| Europe | Italy | 875926 | Respiratory: Sputum | Medicine General |
| Europe | Italy | 875931 | Respiratory: Bronchoalveolar lavage | Medicine General |
| Latin | Colombia | 884610 | Respiratory: Endotracheal aspirate | Medicine ICU |
| America | | | | |
| Latin | Colombia | 884619 | Respiratory: Sputum | Surgery General |
| America | | | | |
| North | United | 890567 | Bodily Fluids: Peritoneal | Other |
| America | States | | | |
| Asia | Thailand | 894608 | Respiratory: Sputum | Medicine ICU |
| Asia | China | 896832 | Respiratory: Sputum | Medicine General |
| Latin | Brazil | 900681 | INT: Wound | Surgery General |
| America | | | | |
| Europe | Italy | 918904 | Respiratory: Bronchoalveolar lavage | Medicine General |
| Europe | Italy | 919877 | Respiratory: Sputum | Surgery ICU |
| Europe | Greece | 921044 | Respiratory: Sputum | Medicine General |
| Europe | Turkey | 926871 | Respiratory: Endotracheal aspirate | General |
| | | | | Unspecified ICU |
| Europe | Turkey | 926901 | Respiratory: Sputum | Medicine General |
| Europe | Greece | 927898 | Respiratory: Endotracheal aspirate | General |
| | | | | Unspecified ICU |
| Europe | Greece | 927915 | Respiratory: Endotracheal aspirate | General |
| | | | | Unspecified ICU |
| Europe | Greece | 927952 | Respiratory: Endotracheal aspirate | General |
| | | | | Unspecified ICU |
| Europe | Greece | 927963 | Respiratory: Endotracheal aspirate | General |
| | | | | Unspecified ICU |

### Example 2: Hybridoma Generation Against K. pneumoniae

43816DM (43816ΔcpsBΔWaaL) strain was used to immunize mice with the goal to elicit antibodies against antigens different from capsule or LPS O-antigen. After the initial phase of immunization with mutant strain, a final boost was performed with a combination of wild-type strains (1901 and 1899) before spleens and lymph nodes were collected for hybridoma generation. Whole-cell bacterial screening by binding was initially applied in hybridoma generation similarly as the above phage panning approach to identify cross-reactive antibodies. Of the approximately 9000 hybridomas tested, four hybridomas (21G10, 22B12, 88D10, and 89E10) showed serotype independent binding to the K. pneumoniae strains tested (FIGS. 1A and E).

### Example 3: Demonstration of Serotype Independent Opsonophagocytic Killing (OPK) Activity

Antibodies with OPK activity have been reported to correlate with in vivo protective function. See. e.g., DiGiandomenico, et al., J Exp Med, 209:1273-87 (2012), herein incorporated by reference. A high throughput OPK assay to facilitate phenotypic screens was adapted. Approximately 1000 hybridomas were maintained in antibiotic free media and tested for OPK activity. The OPK positive hybridomas were then cloned and expanded for antibody purification. Among these, two hybridoma derived antibodies (88D10, 89E10) displayed enhanced OPK activity (FIG. 1B) and showed strong bindings to the K. pneumoniae strain by whole bacteria ELISA assays (FIG. 1A).

OPK positive phage and hybridoma-derived antibodies were also tested for binding to a selective panel of K. pneumoniae strains with various capsule and O-antigen serotypes by ELISA (FIG. 1E). The phage and hybridoma-derived antibodies showed similar binding patterns, and all bound to multiple capsule and O-antigen serotypes.

The ability of the phage-derived Kp3 antibody to bind to ex vivo grown Klebsiella was also tested. In these experiments, Klebsiella strains were cultured in 2xYT broth overnight at 37° C., 250 rpm. The cultures were then diluted 1:200 and allowed to grow to log phase. 5e8 CFU bacteria were injected to mouse via intraperitoneal (Er vivo IP) or intranasal (Ex vivo IN) route. After two hours, mice were sacrificed, and bacteria were isolated from lung homogenate, peritoneal wash, or blood. Bacteria isolated under these conditions were subjected to a FACS binding assay using Kp3. As shown in Table 6, below, Kp3 also binds to multiple Klebsiella serotypes grown ex vivo (“+ or ++ or +++” indicate level of binding; “−” indicates no binding).

TABLE 6
Kp3 binds to Klebsiella grown ex vivo

| Klebsiella Strains | Growth condition | KP3 |
|---|---|---|
| 9178 (O3:K58) | 2xYT broth | ++ |
| | Ex vivo IP | ++ |
| | Ex vivo IN | + |
| 29011 (O1:K2) | 2xYT broth | +++ |
| | Ex vivo IP | + |
| 9148 (O2:K28) | 2xYT broth | ++ |
| | Ex vivo IP | + |
| | Ex vivo IN | + |
| 5046 (O2:K3) | In vitro | − |
| | Ex vivo IN | − |
| 9177 (O5:K57) | In vitro | ++ |
| | Ex vivo IP | + |
| | Ex vivo IN | + |
| 3048554 (KPC) | 2xYT broth | ++ |
| | Ex vivo IP | ++ |

### Example 4: Identification of MrkA Antigen

The similar binding patterns of the two phages (Kp3 and Kp16) and the four hybridoma clones (88D10, 89E10, 21G10, and 22B12) (see FIG. 1E) prompted investigation of the possibility that they recognize the same antigen. In these competition ELISA experiments, 1 μg/ml of biotin-labeled antibody (Kp3 in FIG. 1C or 88D10 in FIG. 1D) was mixed with increasing amounts of Kp3 or Kp16 (as indicated in FIG. 1) and tested for its binding to K. pneumoniae. Anti-mouse-IgG-HRP was used as the detecting agent. The reduction in ELISA signal was expressed as a percentage of inhibition. The competition ELISA showed that they all competed with each other in binding to the K. pneumoniae isolates tested, indicating that they bind to overlapping epitopes on the same antigen (FIGS. 1C and 1D).

Whole-cell protease treatment prior to binding analysis eliminated reactivity of mAbs KP3 and 88D10. This indicates that the target of these antibodies was likely to be a protein. It was also confirmed that the antigen target was located on the surface of K. pneumoniae by confocal microscopy using Kp3 staining, as protruding fibrous cell surface structures resembling fimbriae were visualized (FIG. 2A).

Immunoprecipitation was then used to isolate the mAb-binding antigen target. In these experiments, cell lysate was prepared from non-reactive (1899) and reactive (43816DM) strains and subjected to immunoprecipitation by Kp3, 88D10, and an isotype antibody control. The immunoprecipitation products were divided into two halves and separated on two 4-12% SDS-PAGE gels under reducing conditions. One gel was analyzed by blue stain. The other identical gel was transferred to a PVDF membrane and subjected to Western analysis using a mixture of biotinylated 88D10.1 and Kp3 as the detecting antibodies.

Compared to the control antibody, Kp3 and 88D10 captured four major protein bands with band 1 from a negative control isolate 1899 (FIG. 2B). Among them, band number 3 is reactive to Kp3 in a Western blot analysis (FIG. 2C). All four bands were excised and subjected to LC-MS analysis. The most dominant protein band (FIG. 2B band #3) was identified as MrkA, as peptides covering more than 50% of the full MrkA sequence were recovered. MrkA peptides identified through mass spectrometry are shown in bold face and underlined in FIG. 2D. The other dominant band (FIG. 2B band #2) was identified as MrkB, a chaperon protein that facilitates MrkA fimbrial subunit folding and transportation through the periplasmic space. (Chan et al., Langmuir 28:7428-35 (2012); Burmolle et al., Microbiology 154:187-95 (2008).) The least dominant band (FIG. 2B band #4) and one isolated from the negative control isolate (FIG. 2B band #1) did not identify any specific cell surface localized protein.

### Example 5: Confirmation of MrkA as the Antigen

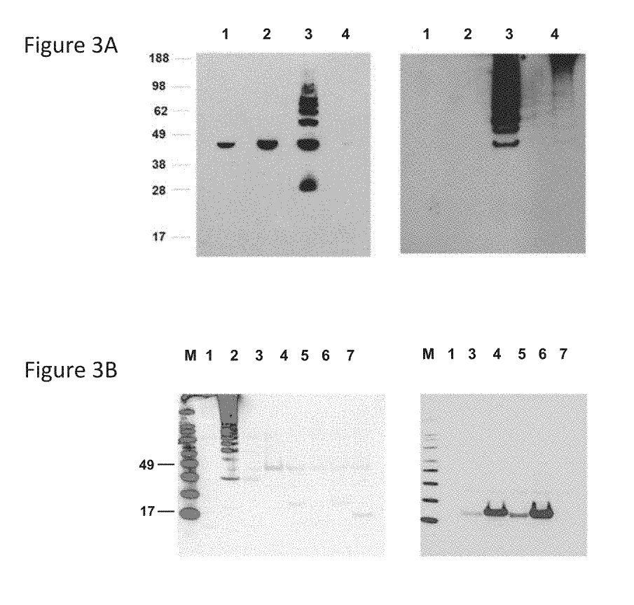

Though MrkA was the single protein species identified from FIG. 2B band No. 3 by LC-MS, there was clear discrepancy between the predicted MW of MrkA (˜20 kDa) and the apparent MW by SDS-PAGE (60-200 kDa) (FIGS. 2B and C). The laddered appearance of bacterial surface protein has been reported previously, including alpha protein C in Group B Streptococci and MrkA. See Chan et al., Langmuir 28:7428-35 (2012) and Langstraat et al., Infect. Immun. 69:5805-12 (2001). To further confirm the identity of the antigen, recombinant MrkA was expressed in E. coli based on the published MrkA sequence of K. pneumoniae MGH78578. Specifically, the MrkA ORF of strain MGH78578 from the UniProt database was cloned into an expression vector and expressed in BL21 cells. Lysates were then prepared using B-PER and subjected to Western blot analysis using an anti-his tag or Kp3. Similar to the endogenous MrkA, the recombinant MrkA displayed a laddered pattern including bands ranging in apparent sizes from 60 kDa to more than 200 kDa (FIG. 3A). Interestingly, while the anti-his antibody recognized both monomeric and oligomeric MrkA, Kp3 recognized only the oligomeric form. The MrkA mAb target identity is also consistent with the fimbriae structure shown in confocal experiments (FIG. 2A). Recombinant MrkA was also expressed with a c-Myc tag in an in vitro transcription and translation system under different experiment conditions, and the products were subjected to Western blot analysis. As indicated by anti-Myc detection, in vitro expression system predominantly produced MrkA monomeric protein (FIG. 3B). While Kp3 recognized higher molecular weight bands present in bacterial cell lysate (FIG. 3B, sample 1), it was not able to detect the MrkA monomer. This suggests that Kp3 binds to high order MrkA structures in type III fimbriae and that the MrkA assembly may require the contribution of other cellular components or conditions which are lacking in the in vitro expression system used in this study.

### Example 6: Anti-MrkA Antibodies Protect Mice Against K. pneumoniae In Vivo

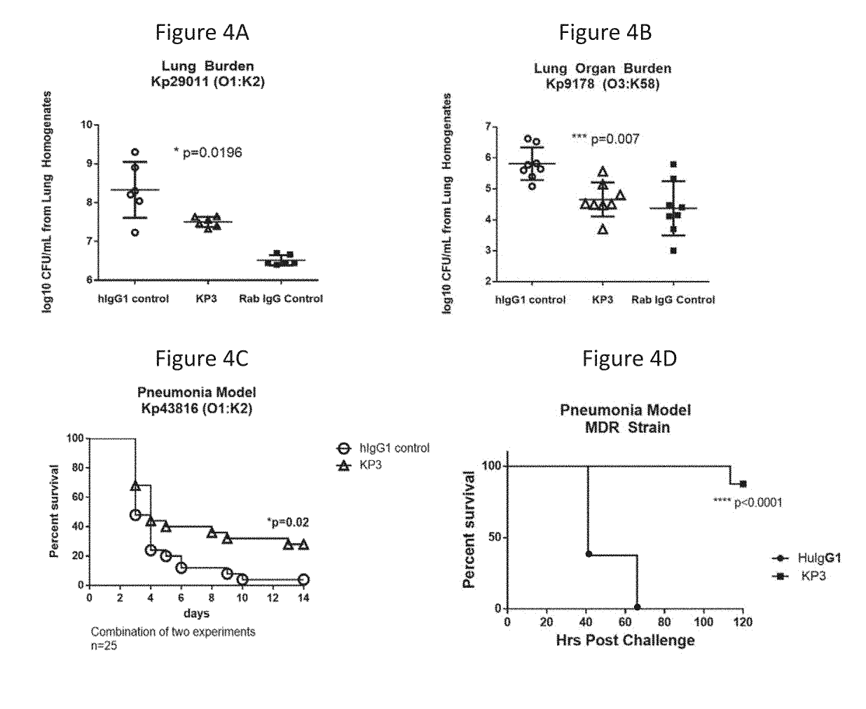

Given the superior serotype independent OPK activity and biofilm prevention by the anti-MrkA antibodies disclosed herein, Kp3 was evaluated in a murine model of K. pneumoniae infection with two major O-serotype strains. The virulence of the different clinical K. pneumoniae isolates varies dramatically in immunocompetent mice. The majority of isolates evaluated were not virulent even at high inoculating doses (1e9 CFU/mouse) in acute pneumonia models with few exceptions. Therefore, an organ burden model was adopted to demonstrate the efficacy of the anti-MrkA antibodies against multiple isolates. In these experiments, mice received a single dose of antibody by IP administration 24 hours prior to intranasal infection with 1e7 CFU of the bacteria. Mice were then sacrificed, and the bacterial counts in the infected lungs were assessed. Kp3 at 15 mg/kg (mpk) significantly reduced lung burden in mice that were infected with Kp29011 (O1:K2) and Kp9178 (O3:K58) (FIGS. 4A and 4B). A human IgG1 rabbit polyclonal antibody against Kp43816 was used as a control. Antibody dose titration showed that 15 mpk gave the best protection, with higher doses producing no additional benefit.

Kp3 was also tested in a lethal pneumonia model using Kp43816, a virulent O1:K2 strain (FIG. 4C) or Kp985048, a recently isolated clinical multi-drug resistant strain (FIG. 4D). In this model, 5e3 CFU (Kp43816) or 1e8 CFU (Kp985048) of the bacteria were given intranasally 24 hours after antibody administration. Mice were monitored up to 8 days post-infection. MAb Kp3 demonstrated significant protective benefit in these models (FIGS. 4C and 4D).

These data indicate that the OPK activity of anti-MrkA antibodies may contribute to their ability to reduce bacterial burden and enhance survival of mice infected with multiple serotypes of K. pneumoniae.

### Example 7: Identification of MrkA Epitope

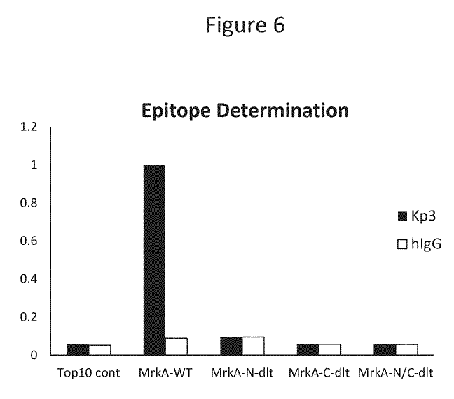

In order to generate MrkA deletions, MrkA gene sequences with a 40 amino acid N-terminal deletion (“MrkA-N-dlt”), a 32 amino acid C-terminal deletion (“MrkA-C-dlt”), and combination of the N- and C-terminal deletions (“MrkA-N/C-dlt”) were cloned into the pCABNTAB6 (GE Healthcare) bacterial expression vector with a His tag added at the C termini. A single colony was picked and inoculated into LB supplemented with 100 units Carbenicilin. The bacteria were cultured at 250 rpm, 37° C. When the OD600 reached 04-0.6, IPTG was added to a final concentration of 1 mM, and the bacteria were cultured for another 3 hours. Bacterial cells were then collected and subjected to lysis using B-PER Bacterial Protein Extraction Reagent (Thermo Scientific). The clear cell lysate was used directly to coat an ELISA plate, and binding of Kp3 was measured using a standard ELISA procedure. Human IgG1 and an unrelated anti-MrkA antibody were used as controls. As shown in FIG. 6, Kp3 only detected full length MrkA and did not bind to: MrkA-N-dlt; i.e., amino acids 41-202 of SEQ ID NO:17 (i.e., SEQ ID NO:26); MrkA-C-dlt; i.e., amino acids 1-170 of SEQ ID NO: 17 (i.e., SEQ ID NO:27) or MrkA-N/C-dlt; i.e., amino acids 41-170 of SEQ ID NO: 17 (i.e., SEQ ID NO:28). In contrast, a control anti-MrkA antibody detected full length MrkA as well as MrkA with N terminal deletion (data not shown). These results show that Kp3 recognizes a conformational epitope.

### Example 8: Monomeric and Polymeric MrkA Reduce Organ Burden in a Bacterial Challenge Model

Given the serotype independent protective activities of anti-MrkA mAb in prophylactic treatment, the ability of purified MrkA to confer protection as a vaccine antigen was tested. Recombinant MrkA protein exists in both monomeric and polymeric form (FIG. 3A). In order to assess the role of monomeric and oligomeric MrkA protein in inducing protective immunity, both monomeric and polymeric species were purified by column fractionations. Briefly, in order to express MrkA on a large scale, the mature form of MrkA (SEQ ID NO: 17) was cloned into pET28 (Novagen) in frame with an N terminal 6× his tag. The protein was expressed by the host BL21-DE3 E. coli strain. Transformed cells were grown in Terrific Broth (Corning)+Kanamycin (50 μg/ml) at 37° C. with 250 RPM shaking until reaching an OD600 of 0.6. IPTG (1 M) (InVitrogen) was added to the culture for a final concentration of 1 mM, and the culture was incubated for an additional 4 hours. The cells were harvested by centrifugation (12,000×g for 10 minutes), and the cell pellet was stored at −80° C. until purification. For MrkA purification, the cell pellet was thawed on ice, lysed using B-PER and the insoluble inclusion body fraction was collected by centrifugation and re-suspended in solubilization buffer (10 mM Tris, pH 8, 100 mM sodium phosphate, 8 M Urea, 1 mM DTT). Solubilized inclusion bodies were clarified by centrifugation at 27,000×g for 15 minutes at 10° C. then loaded onto a 5 ml HisTrap HP column (GE Healthcare) equilibrated with solubilization buffer. Both flow through and eluted fractions were collected and subjected to refolding according to the described protocol. Refolded mixtures were loaded onto a HisTrap column and eluted with a linear gradient up to 500 mM Imidazole in 25 mM sodium phosphate, pH 7.4 with 500 mM NaCl. Monomeric MrkA was collected early in the gradient (approx. 150 mM Imidazole) and oligomeric species later in the gradient (approx. 250 mM Imidazole). Each pool was concentrated with Vivaspin 5 K MWCO devices (Vivascience) and dialyzed into 10 mM Tris, pH 7.5 with 100 mM NaCl.

In order to refold by dialysis, samples were diluted with 3 volumes of Dilution Buffer [10 mM Tris, 100 mM sodium phosphate, 1 mM EDTA, 5 mM Cysteamine, 0.5 mM Cystamine, pH 8]. They were allowed to mix overnight at 4° C. They were dialyzed into refolding buffer (Dilution Buffer without EDTA) at 4° C. (two exchanges) then into 1×PBS, pH 7.2. The dialyzed samples were purified using HisTrap (eluted with a linear gradient to 500 mM Imidazole in 25 mM sodium phosphate, pH 7.4 with 500 mM NaCl).

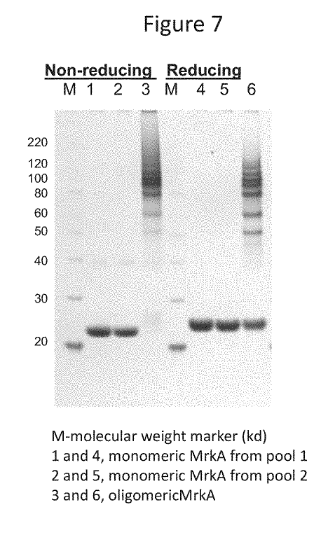

The MrkA that was retained in the column during the first loading step contained mostly oligomeric MrkA. It was refolded on the column, eluted, and concentrated as described above. Purification from inclusion body resulted in monomeric and oligomeric MrkA with high purity (FIG. 7), which was used in subsequent immunization experiments.

The purified and concentrated monomeric and oligomeric MrkA were used to vaccinate mice. Six to eight week old C57/bl6 mice were vaccinated three times through the subcutaneous injection of 15 microgram of monomeric or polymeric MrkA with Freund's adjuvant. After the third infection, strong serum titer against MrkA was detected. Mice were then challenged with 1.4e7 CFU Kp29011 (O1:K2) intra-nasally after the third immunization (week 4). 24 hours post infection, lung and liver were homogenized in 1 mL of PBS and plated on LB agar plates to calculate CFU/mL of homogenate.

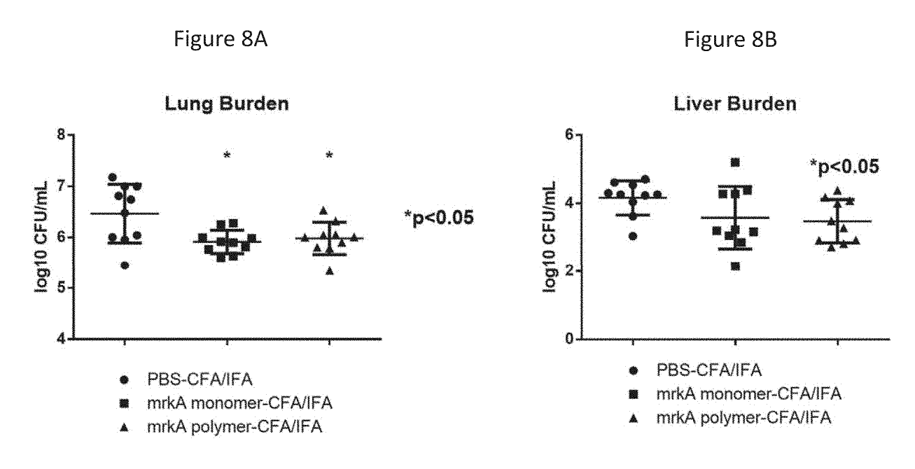

The results, which are shown in FIG. 8, demonstrate that compared with adjuvant control group (PBS-CFA/IFA), both monomeric and oligomeric MrkA vaccination reduced organ burden after bacterial challenge, suggesting that MrkA could confer protection as a vaccine antigen. Monomeric MrkA significantly reduced bacteria in the lung, and oligomeric MrkA significantly reduced bacteria in both the lung and liver (FIG. 8A-B). Thus, these results demonstrate that vaccination with monomeric and/or oligomeric MrkA reduce Klebsiella organ burden.

### Example 9: Anti-MrkA Antibodies Inhibit Biofilm Formation and Cell Attachment

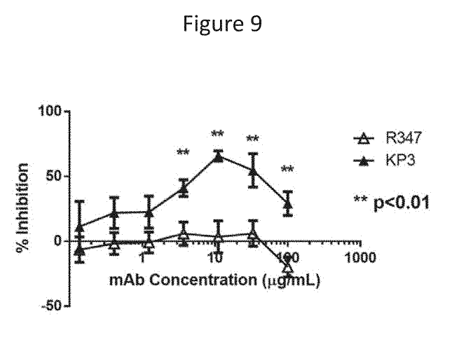

In order to determine if anti-MrkA antibodies inhibit biofilm formation, biofilm assays were performed according to Wilksch et al., (PLos Pathogens 7(8): e1002204 (2011)) with modifications. K. pneumoniae 43816 were allowed to grow into log phase culture and diluted into minimum media (RPMI-1% BSA) to be OD₆₅₀ equals to 0.1. In triplicate, bacteria were incubated in flat bottom, 96 well microtiter plates (Falcon; BD Biosciences) with a series dilution of Kp3 or hIgG1 (isotype control) antibodies. Following 2 h incubation at 37° C., 120 rpm, planktonic bacteria were washed out, and wells were washed with distilled water. Biofilms attached to the well surfaces were stained for 15 min at room temperature with 150 μL of 0.1% (wt/vol) crystal violet solution. The crystal violet solution was decanted, and wells were subsequently washed to thoroughly remove unbound dye. The bound dye were solubilized with 200 μl 95% Ethanol and quantified by measuring absorbance at 595 nm. Wells containing growth media along were used as negative controls to calculate percentage of the inhibition. The ability of bacteria to colonize host tissues or abiotic surfaces, form microcolonies, communities or biofilms plays an important role in pathogenesis and persistence of the bacterial infections. Gupta et al., “Biofilm, pathogenesis and prevention—a joumey to break the wall: a review.” Arch Microbiol. 2015 Sep. 16. Type III fimbriae in K. pneumoniae are filamentous appendages that mediate adherence to eukaryotic cells and abiotic surfaces. MrkA, a major fimbrial subunit, but not adhesin (MrkD) were previously shown to facilitate biofilm formation (Langstraat et al., Infect Immun 2001; 69:5805-12). To determine whether the anti-MrkA antibodies bind to MrkA on the bacterial surface and subsequently block biofilm formation, bacterial attachment to abiotic plate in the presence of anti-MrkA mAb Kp3 or a human IgG1 control antibody was measured. Kp3 significantly blocked biofilm formation by Klebsiella 43816 strain in a dose dependent manner (FIG. 9). Thus, the results shown in FIG. 9 demonstrate that anti-MrkA Kp3 antibody inhibits Klebsiella biofilm formation.

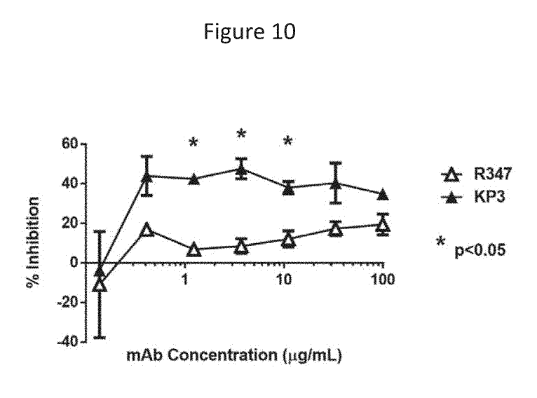

Another important feature of the type III fimbriae is to facilitate Klebsiella colonization of host tissues leading to establishment of infection. To test whether anti-MrkA mAb Kp3 prevented Klebsiella association with lung epithelial cells cell attachment assays were also performed. Briefly, in these experiments, Kp3 or hIgG1 (isotype control) antibodies were added to confluent A549 cells grown in opaque 96-well plates (Nunc Nunclon Delta). Log-phase luminescent K. pneumoniae 43816 was added at a multiplicity of infection (MOI) of 50. After incubation at 37° C. for 90 min, cells were washed, followed by the addition of 0.05 ml of 2xYT+0.5% glucose. Bacterial RLUs were quantified using an Envision Multilabel plate reader (PerkinElmer) after a 15-min incubation at 37° C. As shown in FIG. 10, Kp3 significantly reduced the attachment ofK pneumoniae to A549 human pulmonary epithelial cells thereby demonstrating that anti-MrkA Kp3 antibody inhibits Klebsiella binding to epithelial cells.

### Discussion

A target agnostic strategy was applied to identify cross-protective antibodies for the treatment of K. pneumoniae infection. While significant efforts have been made to identify cross-reactive antibodies targeting K. pneumoniae, there are major obstacles in developing such therapeutics. Well validated antibody targets including CPS and LPS are serotype specific and therefore require multiple antibodies for broad strain coverage. This challenge was overcome by constructing CPS and LPS O-antigen deletion mutants to focus on more conserved surface antigens. By utilizing whole bacterial binding and higher throughput OPK assays, anti-MrkA antibodies from both hybridoma and phage display platforms demonstrating significant in vitro and in vivo efficacies against Klebsiella were identified.

MrkA is a major protein of the type III fimbriae complex and has been implicated in host cell attachment and biofilm formation (see Murphy et al., Future Microbiol 2012; 7:991-1002), a strategy bacterial pathogens use to establish infection (Burmolle et al., Microbiology 2008; 154:187-95). In one proof of concept experiment, mice immunized with purified type III fimbriae displayed resistance to subsequent K. pneumoniae challenge, albeit only to relatively low challenging doses (Lavender et al., International journal of medical microbiology 2005; 295:153-9). Although humoral immunity was implicated as the protective mechanism, the antigenic components that elicited protection were not elucidated. The anti-MrkA monoclonal antibodies disclosed herein contribute to the immune protection through multiple mechanisms. First, anti-MrkA mAbs reduced bacterial attachment to pulmonary cell lines and formation of biofilms, which may subsequently reduce bacterial colonization to host tissues and facilitate bacterial clearance. Second, anti-MrkA mAbs showed strong enhancement of OPK activity independent of serotypes. The OPK activity may assist to reduce the bacterial burden and enhance survival in mice infected with multiple serotypes of K. pneumoniae. Interestingly, antibodies against type III fimbrial adhesin protein MrkD showed cross-reactivity to multiple K. pneumoniae strains similar to anti-MrkA mAbs, but did not induce OPK and confer no protection in vivo (data not shown). This further confirmed that OPK activity may be necessary for in vivo protection for these antibodies.

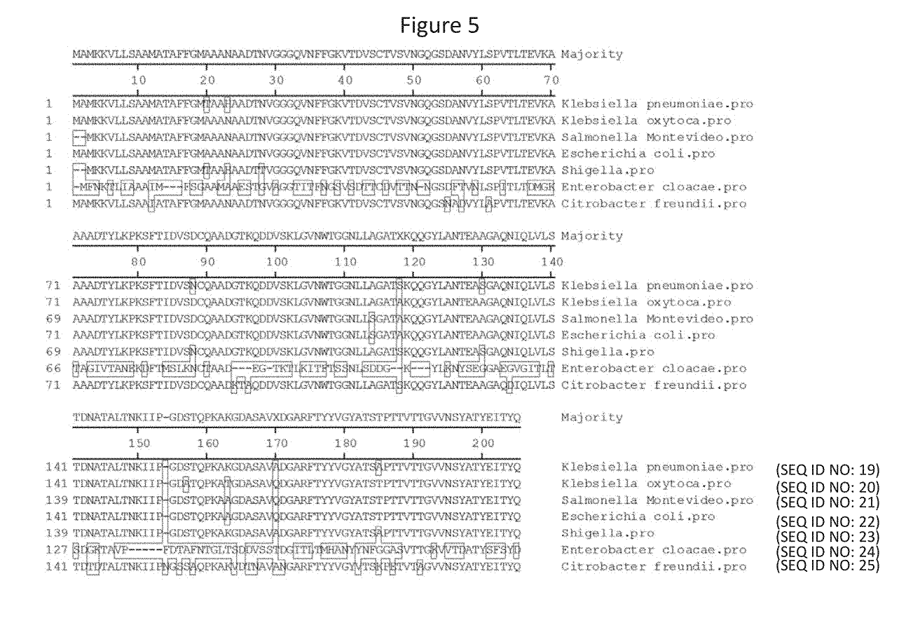

A promising feature of MrkA as an antibody therapeutic target is its high degree of sequence conservation among different isolates and general accessibility as an extracellular target. MrkA from the two most dominant pathogenic isolates K. pneumoniae and K. oxytoca have a 95% homology, and the homologies among representative members of the Enterobacterecea family are more than 90% with the exception of Enterobacter cloacae, which is divergent from the rest (FIG. 5). Further work is needed to survey extensively the MrkA sequences from other members. Nevertheless this presents a potential opportunity to develop a MrkA-based anti-K. pneumoniae and pan Gram negative strategy.

It is noteworthy that anti-MrkA antibodies isolated from two different platforms converge in targeting similar epitopes. This is in stark contrast to a recent report showing that antibodies resulting from hybridoma and phage campaigns targeted divergent epitopes (Rossant et al., mAbs 2014; 6:1425-38). The epitopes appear to be conformational in nature. It is consistent with the findings that the identified functional antibodies disclosed herein recognize an epitope that exists predominantly on oligomeric MrkA. Vaccination studies with purified monomer and multimeric MrkA antigens suggested that antigen in both forms can induce protective immunity. These observations may have important implications for MrkA based therapeutics and vaccine development.

In summary, these studies further demonstrate that functional screening of antibodies is a powerful tool in therapeutic development and new target discovery against K. pneumoniae. The wealth of information generated from this study surrounding MrkA and anti-MrkA antibodies should be useful to the field of K. pneumoniae pathogenesis and add to the arsenal in fighting against K. pneumoniae and other severe bacterial infections.

### Example 10: Phage Library Panning Against Recombinant MrkA Protein

Additional anti-MrkA antibodies were identified by panning naïve human single-chain variable fragment (scFv) antibody phage libraries against purified recombinant MrkA protein.

In order to prepare recombinant MrkA protein, his-tagged recombinant MrkA was expressed and purified as described in the materials and methods section with modifications. MrkA expressed in the E. coli host strain BL21(DE3) stayed mostly in the inclusion body. Buffer containing eight molar urea was used to solubilize MrkA, and the his-tagged MrkA was purified using HisTrap HP column (GE Healthcare) as described previously (see Wang. Q. et al, 2016. Target Agnostic Identification of Functional Monoclonal Antibodies Against Klebsiella pneumoniae Multimeric MrkA Fimbrial Subunit. Journal of Infectious Diseases, 213 (11): 1800-1808, herein incorporated by reference), with the exception that denatured MrkA was loaded directly to the affinity column and purified under the denaturing condition without refolding first. Monomeric and oligomeric MrkA were eluted together without further separations. Eluted MrkA fractions were collected and dialyzed against PBS buffer and were then ready for biotin labeling and panning. For biotin-labeling, the labeling kit from Pierce was used, and the manufacturer's protocol was followed.

Panning selection was performed in a solution format using a Kingfisher automated system as described in Lillo, A. M. et al. (“Development of phage-based single chain Fv antibody reagents for detection of Yersinia pestis,” PLoS One 6:e27756 (2011)) with modifications. Naïve scFv phage display libraries used in this study were described previously in Vaughan T. J. et al. (“Human antibodies with sub-nanomolar affinities isolated from a large non-immunized phage display library,” Nat Biotechnol 14:309-314 (1996)). Panning antigen MrkA was biotinylated, and 0.3 μg was used in each of the first two rounds of panning. For selections that needed a third round, biotinylated MrkA was reduced to 0.1 μg. When the phage output was improved to more than 100-fold compared to that of the first round, panning selection was stopped and high throughput screenings were initiated.

The first round of screening was based on specific bindings to MrkA. scFv.Fc expressed through the pSplice.V5 vector in E. coli strain Top 10 (Invitrogen) was used in a homogeneous time resolved FRET (HTRF) based assay to screen for specific binders. (Xiao X, et al., “A Novel Dual Expression Platform for High Throughput Functional Screening of Phage Libraries in Product like Format,” PLoS One 10:e0140691 (2015); Newton P. et al., “Development of a homogeneous high-throughput screening assay for biological inhibitors of human rhinovirus infection.” J Biomol Screen 18:237-246 (2013).) Resulting MrkA-specific binders were consolidated and sequenced. The unique clones were used to prepare plasmids for mammalian cell transfection, scFv.Fc expression, and OPK analysis as described previously. (See Xiao X. et al., “A Novel Dual Expression Platform for High Throughput Functional Screening of Phage Libraries in Product like Format,” PLoS One 10:e0140691 (2015)).

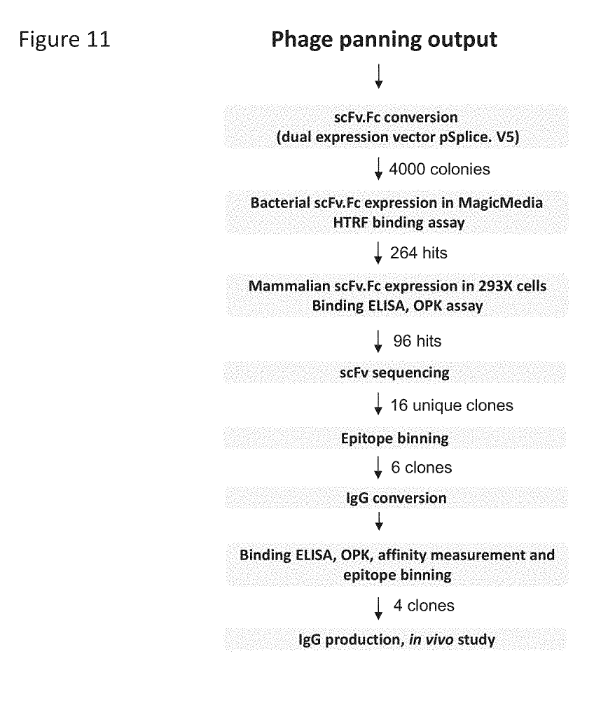

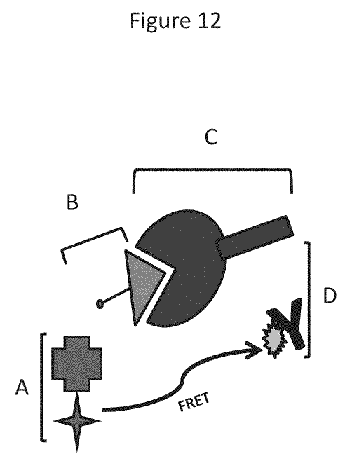

For panning purposes, monomeric MrkA was not separated from oligomeric MrkA. After the second or third round of selection, the panning output was improved more than 100-fold compared to the first round. The panning output was converted to scFv.Fc in pSplice.V5 and subjected to high throughput screening as described above and summarized in FIG. 11, with further illustration of the homogeneous time resolved FRET (HTRF) process in FIG. 12. Starting with more than 4000 colonies, four different MrkA-specific, OPK-positive antibodies that bind to different epitopes were identified. These four antibodies were converted to the human IgG1 format and subjected to further characterizations as described below. They are named anti-MrkA clones 1, 4, 5, and 6.

### Example 11: Characterization of Anti-MrkA Clones 1, 4, 5, and 6

Those scFv.Fc clones showing positive OPK activities were binned based on a bio-layer interferometry (BLI) assay to assess their apparent affinities and relative binding epitopes.

For affinity measurement, two different formats were used. The first used an IgG against a mixture of monomeric and oligomeric MrkA. The second used a Fab against a monomeric MrkA. A ForteBio Octet QK384 instrument was used to study kinetics of the anti-MrkA mAbs. All the assays were done at 200 μl/well in ForteBio 10× kinetic buffer at 30° C. 0.3 μg/ml of biotinylated-MrkA was loaded on the surface of streptavidin biosensors (SA) for 400 seconds reaching levels between 1.0 and 1.5 nm, followed by a 300 second biosensor washing step. Association of MrkA on the biosensor to the individual mAbs in solution (0.274-200 nM) was analyzed for 600 seconds. Dissociation of the interaction was probed for 600 seconds. Any systematic baseline drift was corrected by subtracting the shift recorded for a sensor loaded with ligand but incubated without analyte. Octet Data Analysis software version 8.0 was used for curve fitting with the binding equations available for a 1:1 interaction model. Global analyses were done using nonlinear least squares fitting. Goodness of fit for the data were assessed by the generated residual plots, R2 and χ2 values.

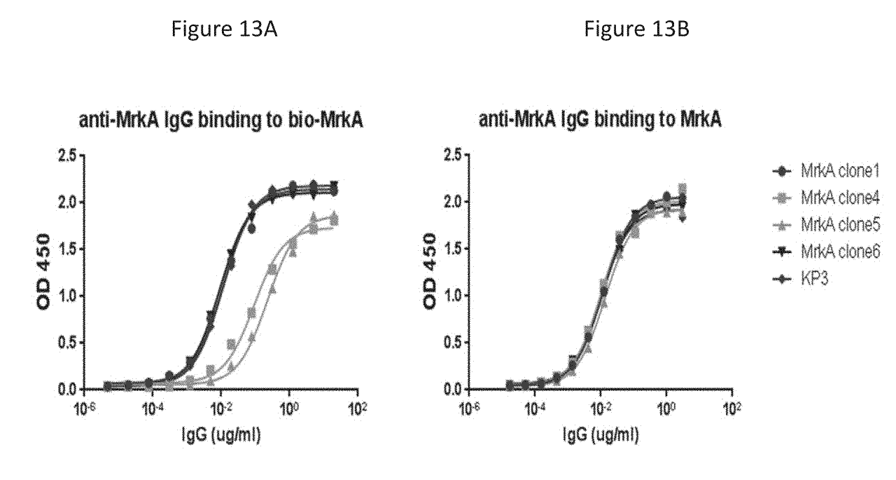

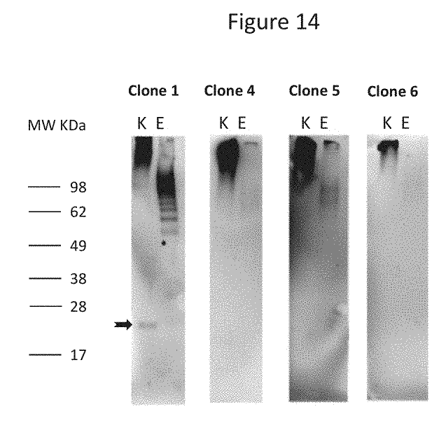

The four clones 1, 4, 5, and 6 were expressed as human IgG1 in 293 free style cells (Invitrogen) and purified. While they maintained robust binding activities, the ELISA format impacted the bindings significantly. Their apparent affinities are between 3-10 nM (FIG. 13 and Table 7) as measured by BLI approach in an IgG format. Western blot data showed that only clone 1 was able to detect the monomeric MrkA, whereas none of the others were able to do so (FIG. 14).

TABLE 7
K_{D} measurement in IgG format against a mixture
of monomeric and multimeric MrkA.

| IgG | K_{D} | Kₒₙ (×10⁴ 1/Ms) | K_{off} (×10⁻⁴ 1/s) | R² |
|---|---|---|---|---|
| clone 1 | 3.25 nM | 5.3 | 1.7 × 10⁻⁴ | 0.989 |
| clone 4 | 3.61 nM | 4.06 | 1.46 × 10⁻⁴ | 0.985 |
| clone 5 | 3.54 nM | 2.6 | 9.2 × 10⁻⁵ | 0.996 |
| clone 6 | 8.80 nM | 2.2 | 2.0 × 10⁻⁴ | 0.996 |
| KP3 | 0.15 nM | 7.6 | 1.2 × 10⁻⁵ | 0.993 |

MrkA is especially intolerant to mutations, and sub-clones and expression of fragments from MrkA often resulted in no expression. Thus, mutational analysis is not a suitable method for epitope analysis. Instead, the BLI-based approach for studying the relative positions of the epitopes of the mAbs was used. Epitope binning was done on a ForteBio Octet QK384. Biotinylated-MrkA was captured onto streptavidin biosensors and coated with testing mAbs at a saturating concentration of 200 nM for 600 seconds. The epitopes of other mAbs were probed in relation to testing mAbs by assaying the testing mAb-coated biosensors in 100 nM each of the other mAbs together with equal concentration of the testing mAb. All graphs were overlaid and aligned at the baseline.

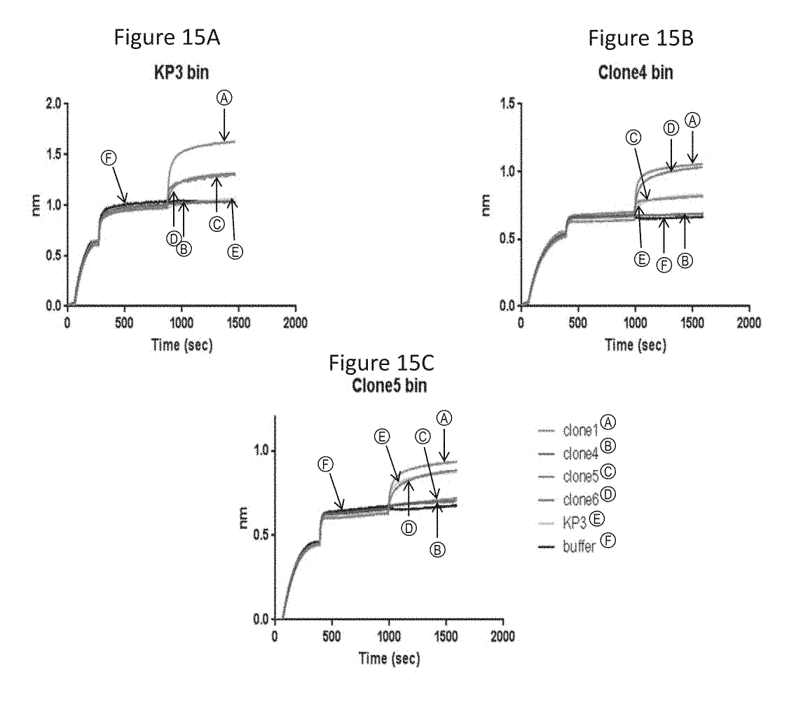

In the binning experiments, IgG clone 1 appears to bind to an epitope that is different from all others, whereas IgG clones 4, 5, 6, and KP3 bound to epitopes that overlap to a limited extent as revealed by different binning setups (FIG. 15). In a peptide scanning experiment, none of the antibodies recognized an overlapping peptide array covering the entire length of MrkA.

When monomeric MrkA was used in a BLI assay against the Fab format of the four clones, it was surprising to find that only clones 1 and 5 retained binding activities to different levels, whereas clone 4 and KP3 lost the bindings entirely (Table 8).

TABLE 8
K_{D} measurement in Fab format against monomeric MrkA.

| Fab | K_{D} | Kₒₙ (×10⁶ 1/Ms) | K_{off} (×10⁻³ 1/s) | R² |
|---|---|---|---|---|
| Clone 1 | 2.76 nM | 0.15 | 0.34 | 0.998 |
| Clone 4 | ND | ND | N/A | — |
| Clone 5 | 1520 nM | 0.05 | 78.2 | 0.997 |
| KP3 | ND | ND | N/A | — |

ND, not detectable;
N/A, not applicable.

These data demonstrate that clones 4, 5, and 6 and KP3 bind to overlapping epitopes on oligomeric MrkA, whereas clone 1 binds to a non-overlapping epitope of MrkA as well as to monomeric MrkA.

### Example 12: OPK Activity is Important for In Vivo Protection

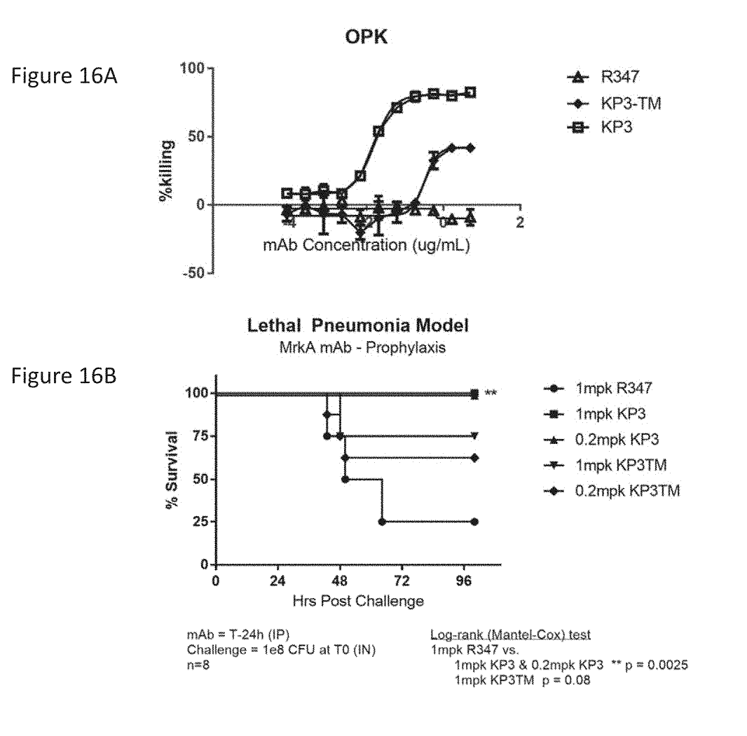

In order to understand the role of OPK activity in in vivo protection, a KP3 IgG was generated. It contained TM mutations to eliminate its effector functions. (Oganesyan V. et al, Acta Crystallogr D Biol Crystallogr 64:700-704 (2008).) The OPK activity was reduced significantly (FIG. 16, top panel), and the reduction in OPK activity corresponded to a reduction in an in vivo prophylaxis protection challenge model. However, neither the OPK activity nor the in vivo protection was completely eliminated (FIG. 16, bottom panel). These data indicate that OPK is important to the protective mechanism of the anti-MrkA antibody KP3.

### Example 13: Antibody Binding to Live Bacteria as Exemplified by Flow Cytometry

To determine whether the clones bind to K. pneumoniae “KP,” flow cytometry analysis was performed against live bacteria of different serotypes. In these assays, bacteria were cultured in 2xYT broth overnight and then diluted into FACS buffer (PBS with 0.5% of Bovine Serum Albumin) to an approximate concentration of 2e7 CFU/mL. Bacteria (1e6) were incubated with anti-MrkA antibodies or with negative control antibody for 1 hour at 4° C. with gentle shaking. Plates were washed with FACS buffer and centrifuged (3500 rpm, 5 min), followed by incubation with Alexa Fluor 647 goat anti-human IgG secondary antibody (Life Technologies). Plates were incubated in the dark for 1 hour at 4° C. with gentle shaking and washed twice with FACS buffer. Samples were measured in a BD LSR II (BD Biosciences) and analyzed using FlowJo.

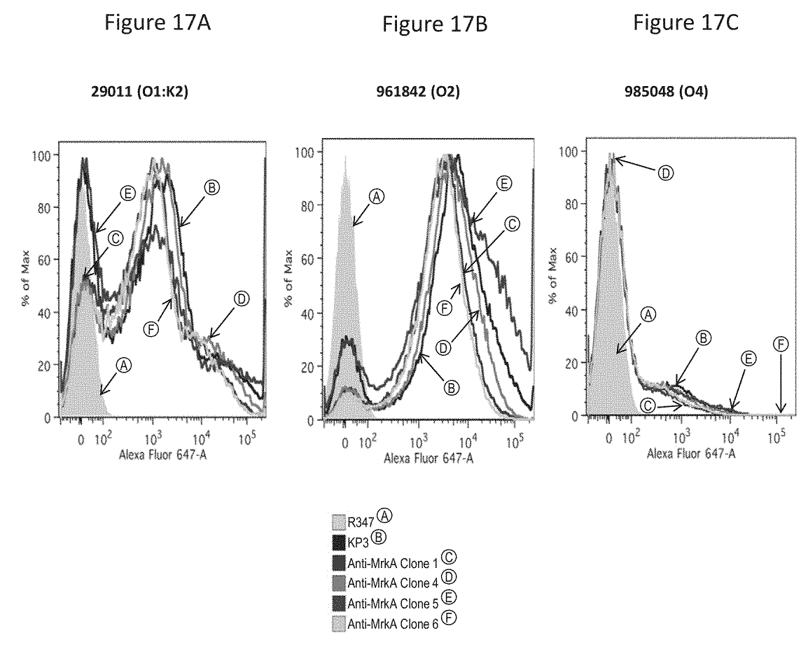

All four clones 1, 4, 5, and 6 recognized the three isolates tested. Even though there were clear differences in binding patterns to different isolates by each antibody, there were no significant differences among the antibodies (FIG. 17). Furthermore, selected isolates were inoculated by intranasal route, and bronchalveolar lavage was collected three hours post infection. The anti-MrkA antibody binding to these in vivo passaged bacteria was then analyzed. The results confirmed that anti-MrkA mAbs bound to the in vivo grown bacteria in a similar fashion as the in vitro culture grown bacteria. In sum, the anti-MrkA antibodies positively bound to a wide collection of KP isolates.

### Example 14: Antibody Characterization by OPK Assay

In order to characterize OPK activity, representative clones from each binning group including clones 1, 4, 5, and 6 were converted to IgG1, expressed, purified and analyzed in an OPK assay as described previously. Briefly, log phase culture of luminescent KP strains (Lux) was diluted to ˜2×10⁶ cells/ml. Bacteria, diluted baby rabbit serum providing complement (Cedarlane, 1:10), dimethylformamide (DMF), differentiated HL-60 cells or freshly isolated polymorphonuclear leukocytes (PMN) cells, and anti-MrkA IgGs were mixed in 96-well plates and incubated at 37° C. for two hours with shaking (250 rpm). The relative light units (RLUs) were then measured using an Envision Multilabel plate reader (Perkin Elmer). The percentage of killing was determined by comparing RLU derived from assays with no antibodies to RLU obtained from anti-KP mAbs and a negative control mAb.

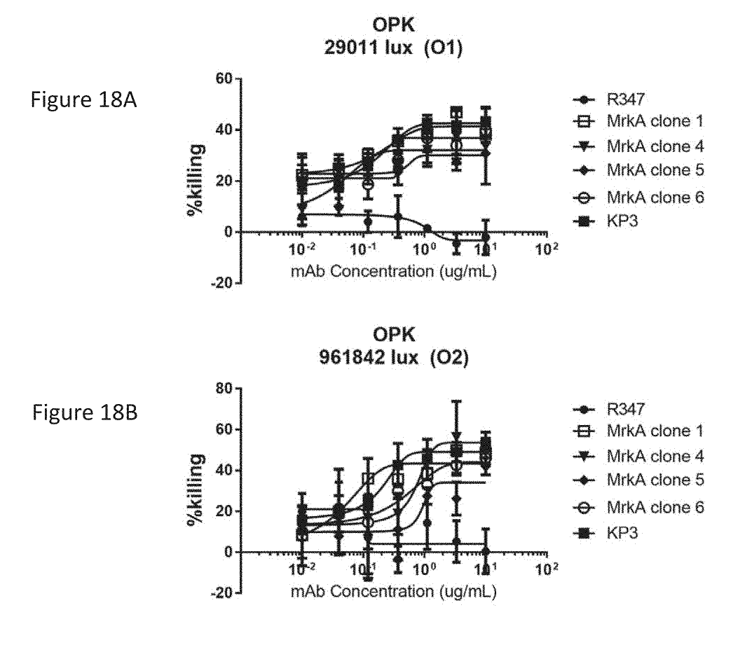

Clones 1, 4, 5, and 6 were selected for further analysis due to their different epitopes and their positive OPK activity in the scFv-Fc format during the screening process. OPK analysis was performed with their IgG1 counterparts, and they all displayed potent OPK activity comparable to that of KP3 against KP of different serotypes. (FIG. 18.) Thus, anti-MrkA antibodies have potent OPK activity against multiple KP serotypes.

### Example 15: Antibody Protective Effects in an In Vivo Challenge Model

In order to evaluate the in vivo protective activities of anti-MrkA antibodies, an acute pneumonia model was used. C57BL/6 mice were inoculated with 1-2e8 CFU of a multi-drug resistant isolate intranasally. KP3, a human IgG control antibody R347, and clones 1, 4, 5, and 6 antibodies were given via intraperitoneal (IP) route either 24 hour prior to bacterial challenge for prophylaxis or one hour post bacterial challenge for therapy. Mouse survival was monitored daily for a minimum of five days until up to day 8. Survival data of representative experiments was plotted in Prism.

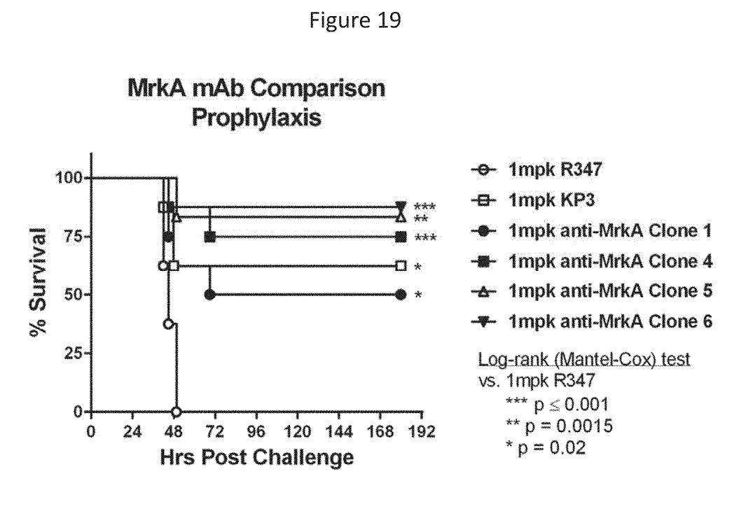

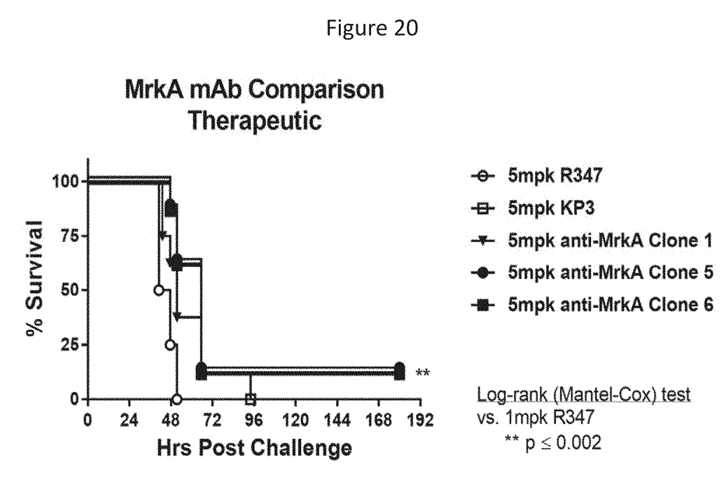

Reflecting their comparable bacterial binding and OPK activity, all of clone 1, 4, 5, and 6 antibodies displayed similarly potent in vivo protective activities in the prophylaxis model (FIG. 19). At 1 mg/kg dose, all of clone 1, 4, 5, and 6 antibodies conferred near complete protection. In the therapeutic model, modest protection was seen at a dose of 5 mg/kg (FIG. 20). There did not seem to be significant differences between antibodies targeting different epitopes in their activities in either model.

Surprisingly, dose response did not always hold true for all the anti-MrkA antibodies in in vivo protection models, and there was a lack of direct correlation between anti-MrkA antibody binding intensity to the bacteria and their in vivo protective effect. Nonetheless, the anti-MrkA antibodies did show protective activity in m vivo.

### Example 16: Single Antibodies are as Protective as Antibody Combinations

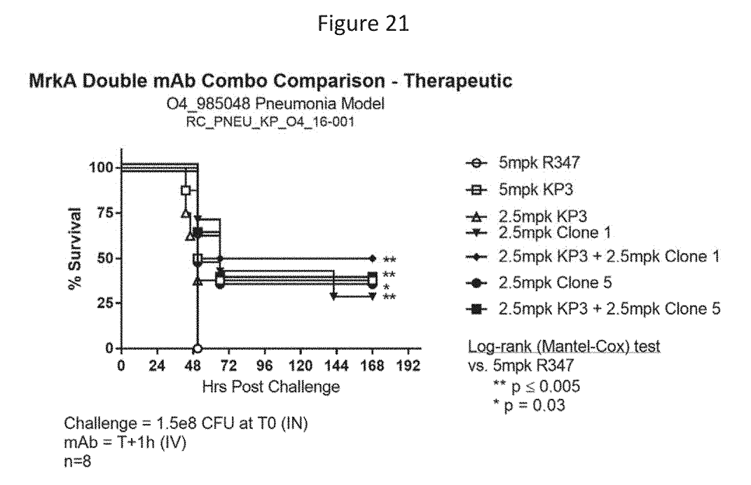

Antibody combinations in the antibacterial field have achieved some very promising results. Thus, combinations of the anti-MrkA antibodies were investigated. Significant additive or synergistic effects were not observed when KP3 was combined with either of clones 1 or 5 (FIG. 21). More complex combinations with up to three mAbs also did not show any additional benefit. Therefore, single anti-MrkA antibodies are as protective as anti-MrkA antibody combinations.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1.-7. (canceled)

8. An isolated antigen binding protein that specifically binds MrkA comprising a set of Complementarity-Determining Regions (CDRs): HCDR1, HCDR2, HCDR3, LCDR1, LCDR2, and LCDR3 wherein:
HCDR1 has the amino acid sequence of SEQ. ID. NO: 1, 4, 29, 32, 35, or 38;
HCDR2 has the amino acid sequence of SEQ. ID. NO: 2, 5, 30, 33, 36, or 39;
HCDR3 has the amino acid sequence of SEQ. ID. NO: 3, 6, 31, 34, 37, or 40;
LCDR1 has the amino acid sequence of SEQ. ID. NO: 7, 10, 41, 44, 47, or 50;
LCDR2 has the amino acid sequence of SEQ. ID. NO: 8, 11, 42, 45, 48, or 51; and
LCDR3 has the amino acid sequence of SEQ. ID. NO: 9, 12, 43, 46, 49, or 52.

9. (canceled)

10. The antigen binding protein of claim 8, wherein said antigen binding protein thereof comprises a VH comprising SEQ ID NO: 13, 14, 53, 54, 55, or 56 and a VL comprising SEQ ID NO: 15, 16, 57, 58, 59, or 60.

11.-19. (canceled)

20. An isolated antigen binding protein that specifically binds to MrkA, wherein the antigen binding protein binds to an epitope in amino acids 1-40 and 171-202 of SEQ ID NO: 17; or binds to MrkA (SEQ ID NO: 17) but does not bind to either SEQ ID NO: 26 or SEQ ID NO: 27.

21.-28. (canceled)

29. The antigen binding protein of claim 8, wherein the antigen binding protein or antigen-binding fragment thereof binds oligomeric MrkA.

30. The antigen-binding protein of claim 8, wherein the antigen binding protein specifically binds to oligomeric MrkA, but does not bind to monomeric MrkA.

31. The antigen binding protein of claim 8, wherein said antigen binding protein is murine, non-human, humanized, chimeric, resurfaced, or human.

32. The antigen binding protein of claim 31, wherein said antigen binding protein is an antibody.

33. The antigen binding protein of claim 32, wherein said antigen binding protein is an antigen binding fragment of an antibody.

34. The antigen binding protein of claim 8, which is a monoclonal antibody, a recombinant antibody, a human antibody, a humanized antibody, a chimeric antibody, a bi-specific antibody, a multi-specific antibody, or an antigen binding fragment thereof.

35. The antigen binding protein of claim 34, wherein said antigen binding protein comprises a Fab, Fab′, F(ab′)2, Fd, single chain Fv or scFv, disulfide linked Fv, V-NAR domain, IgNar, intrabody, IgGΔCH2, minibody, F(ab′)3, tetrabody, triabody, diabody, single-domain antibody, DVD-Ig, Fcab, mAb2, (scFv)2, or scFv-Fc.

36. The antigen binding protein of claim 8, which binds to MrkA with a Kd of about 1.0 to about 10 nM.

37. The antigen binding protein of claim 36, which binds to MrkA with a Kd of 1.0 nM or less.

38. The antigen binding protein of claim 36 wherein the binding affinity is measured by flow cytometry, Biacore, KinExa, radioimmunoassay, or bio-layer interferometry (BLI).

39.-41. (canceled)

42. The antigen binding protein or antibody of claim 32, wherein the antigen binding protein or antibody comprises a heavy chain immunoglobulin constant domain selected from the group consisting of:
(a) an IgA constant domain;
(b) an IgD constant domain;
(c) an IgE constant domain;
(d) an IgG1 constant domain;
(e) an IgG2 constant domain;
(f) an IgG3 constant domain;
(g) an IgG4 constant domain; and
(h) an IgM constant domain.

43. The antigen binding protein or antibody of claim 32, wherein the antigen binding protein comprises a light chain immunoglobulin constant domain selected from the group consisting of:
(a) an Ig kappa constant domain; and
(b) an Ig lambda constant domain.

44. The antigen binding protein or antibody of claim 32, wherein the antigen binding protein comprises a human IgG1 constant domain and a human lambda constant domain.

45. The antigen binding protein or antibody of claim 42, wherein the antigen binding protein comprises an IgG1 constant domain.

46. The antigen binding protein or antibody of claim 32, wherein the antigen binding protein comprises an IgG1/IgG3 chimeric constant domain.

47. A hybridoma producing the antigen binding protein or antibody of claim 8.

48. An isolated host cell producing the antigen binding protein or antibody of claim 8.

49. A method of making the antigen binding protein or antibody of claim 8 comprising (a) culturing a host cell expressing said antigen binding protein or antibody; and (b) isolating said antigen binding protein or antibody from said cultured host cell.

50. An antigen binding protein or antibody produced using the method of claim 49.

51. A pharmaceutical composition comprising the antigen binding protein or antibody according to claim 8 and a pharmaceutically acceptable excipient.

52. The pharmaceutical composition of claim 51, wherein said pharmaceutically acceptable excipient is a preservative, stabilizer, or antioxidant.

53.-57. (canceled)

58. A method for treating, preventing, or ameliorating a condition associated with a Klebsiella infection in a subject in need thereof comprising administering to said subject an effective amount of the antigen binding protein, antibody, or the pharmaceutical composition of claim 51.

59. A method for inhibiting the growth of Klebsiella in a subject comprising administering to a subject in need thereof the antigen binding protein, antibody, or the pharmaceutical composition of claim 51.

60. A method for treating, preventing, or ameliorating a condition associated with a Klebsiella infection in a subject in need thereof comprising administering to said subject an effective amount of an anti-MrkA antibody or an antigen binding fragment thereof.

61. A method for inhibiting the growth of Klebsiella in a subject comprising administering to a subject in need thereof an effective amount of an anti-MrkA antibody or an antigen binding fragment thereof.

62. The method of claim 61, wherein the anti-MrkA antibody or antigen binding fragment thereof specifically binds to K. pneumoniae, K. oxytoca, K. planticola and/or K. granulomatis MrkA.

63. The method of claim 62, wherein the anti-MrkA antibody or antigen binding fragment thereof specifically binds to K. pneumoniae MrkA.

64. The method of claim 60 wherein the condition is selected from the group consisting of pneumonia, urinary tract infection, septicemia, neonatal septicemia, diarrhea, soft tissue infection, infection following an organ transplant, surgery infection, wound infection, lung infection, pyogenic liver abscesses (PLA), endophthalmitis, meningitis, necrotizing meningitis, ankylosing spondylitis, and spondyloarthropathies.

65. The method of claim 64, wherein the condition is a nosocomial infection.

66. The method of claim 65, wherein the Klebsiella is K. pneumoniae, K. oxytoca, K. planticola and/or K. granulomatis.

67. The method of claim 66, wherein the Klebsiella is resistant to cephalosporin, aminoglycoside, quinolone, and/or carbapenem.

68. The method of claim 60, further comprising administering an antibiotic.

69. The method of claim 68, wherein the antibiotic is a carbapanem or colistin.

70. An isolated nucleic acid molecule encoding the antigen binding protein or antibody according to claim 8.

71. (canceled)

72. (canceled)

73. The nucleic acid molecule according to claim 70, wherein the nucleic acid molecule is operably linked to a control sequence.

74. A vector comprising the nucleic acid molecule according to claim 73.

75. A host cell transformed with the the vector of claim 74.

76. (canceled)

77. The host cell of claim 75, wherein the host cell is a mammalian host cell.

78. The mammalian host cell of claim 77, wherein the host cell is a NS0 murine myeloma cell, a PER.C6® human cell, or a Chinese hamster ovary (CHO) cells.

79.-101. (canceled)
